(19) United States
(12) Patent Application Publication
(10) Pub. No.: US 2023/0149127 A1
(43) Pub. Date: May 18, 2023

(54) SYSTEMS AND METHODS FOR GENERATING AN INTERACTIVE VISUALIZATION OF A TREATMENT PLAN

(71) Applicant: SDC U.S. SmilePay SPV, Nashville, TN (US)

(72) Inventors: Sergey NIKOLSKIY, Nashville, TN (US); Ryan AMELON, Coralville, IA (US); Anton Sergeevich ZADORA, Moscow (RU); Stanislav Dmitrievich GROKHOLSKII, Moscow (RU); Tim WUCHER, Windhoek (NA); Jordan KATZMAN, Nashville, TN (US); John SHERIDAN, Nashville, TN (US); Ashley KING, Nashville, TN (US)

(73) Assignee: SDC U.S. SmilePay SPV, Nashville, TN (US)

(21) Appl. No.: 17/883,150

(22) Filed: Aug. 8, 2022

Related U.S. Application Data
(63) Continuation-in-part of application No. PCT/RU21/00513, filed on Nov. 17, 2021.

(51) Int. Cl. A61C 7/00; A61C 9/00

(52) U.S. Cl. CPC A61C 7/002; A61C 9/0053

(57) ABSTRACT

Systems, methods, and computer readable medium of visualizing a treatment of teeth includes capturing, by a user device, a representation representing one or more teeth of a user via a camera of a user device, transmitting, by the user device, the representation to a treatment planning system, receiving, by the user device, a graphical visualization of a treatment plan for moving the one or more teeth of the user from the treatment planning system, the graphical visualization generated based on the representation, displaying, by the user device, the graphical visualization, wherein the graphical visualization comprises a three-dimensional (3D) representation corresponding to the one or more teeth of the user and an interactive object associated with the treatment plan.

### CROSS-REFERENCE TO RELATED APPLICATION

This application is a continuation-in-part of International Application No. PCT/RU2021/000513, filed Nov. 17, 2021, which is incorporated herein by reference in its entirety.

### TECHNICAL FIELD

The present disclosure relates generally to the field of dental treatment, and more specifically to systems and methods for automatically generating three dimensional (3D) teeth positions learned from full 3D teeth geometries, which are used for generating a treatment plan for orthodontic care.

### BACKGROUND

Some patients may receive treatment for misalignment of teeth using dental aligners. To provide the patient with dental aligners to treat the misalignment, a treatment plan is typically generated and/or approved by a treating dentist. The treatment plan may include 3D representations of the patient's teeth as they are expected to progress from their pre-treatment position (e.g., an initial position) to a target, final position selected by a treating dentist, taking into account a variety of clinical, practical and aesthetic factors. Selecting the final position typically involves an arduous process of selecting and moving teeth on an individual basis. Additionally, since the selection is made by a treating dentist, the final position determined or selected by the treating dentist typically involves the dentist's subjective opinion on the best treatment outcome.

### SUMMARY

In one aspect, this disclosure is directed to a method of visualizing a treatment of teeth. The method includes capturing, by a user device, a representation representing one or more teeth of a user via a camera of a user device, transmitting, by the user device, the representation to a treatment planning system, receiving, by the user device, a graphical visualization of a treatment plan for moving the one or more teeth of the user from the treatment planning system, the graphical visualization generated based on the representation, displaying, by the user device, the graphical visualization, wherein the graphical visualization includes a three-dimensional (3D) representation corresponding to the one or more teeth of the user and an interactive object associated with the treatment plan.

In another aspect, this disclosure is directed to a non-transitory computer readable medium storing instructions that, when executed by one or more processors, cause the one or more processors to capture a representation representing one or more teeth of a user via a camera of a user device, transmit the representation to a treatment planning system, receive a graphical visualization of a treatment plan for moving the one or more teeth of the user from the treatment planning system, the graphical visualization generated based on the representation, display the graphical visualization, wherein the graphical visualization includes a three-dimensional (3D) representation corresponding to the one or more teeth of the user and an interactive object associated with the treatment plan.

In another aspect, this disclosure is directed to a non-transitory computer readable medium storing instructions that, when executed by one or more processors, cause the one or more processors to transmit a two-dimensional (2D) representation representing one or more teeth of a user to a treatment planning system, receive a graphical visualization of a treatment plan for moving the one or more teeth of the user from the treatment planning system, the graphical visualization generated based on the 2D representation, display the graphical visualization, wherein the graphical visualization comprises a three-dimensional (3D) representation corresponding to the one or more teeth of the user and an interactive object associated with the treatment plan.

Various other embodiments and aspects of the disclosure will become apparent based on the drawings and detailed description of the following disclosure.

### BRIEF DESCRIPTION OF THE DRAWINGS

FIG. 1 shows a system for orthodontic treatment, according to an illustrative embodiment.

FIG. 2 shows a process flow of generating a treatment plan, according to an illustrative embodiment.

FIG. 3 shows a top-down simplified view of a model of a dentition, according to an illustrative embodiment.

FIG. 4 shows a perspective view of a three-dimensional model of the dentition of FIG. 3, according to an illustrative embodiment.

FIG. 5 shows a trace of a gingiva-tooth interface on the model shown in FIG. 3, according to an illustrative embodiment.

FIG. 6 shows selection of teeth in a tooth model generated from the model shown in FIG. 5, according to an illustrative embodiment.

FIG. 7 shows a segmented tooth model of an initial position of the dentition shown in FIG. 3, according to an illustrative embodiment.

FIG. 8 shows a target final position of the dentition from the initial position of the dentition shown in FIG. 7, according to an illustrative embodiment.

FIG. 9 shows a series of stages of the dentition from the initial position shown in FIG. 7 to the target final position shown in FIG. 8, according to an illustrative embodiment.

FIG. 10 shows a view of a final position processing engine of a treatment planning computing system of FIG. 1, according to an illustrative embodiment.

FIG. 11 shows a block diagram of an example system using supervised learning that may be used to determine a final position of the teeth, according to an illustrative embodiment.

FIG. 12 shows a block diagram of a simplified neural network model, according to an illustrative embodiment.

FIGS. 13A-13D show a system for generating a treatment plan, according to illustrative embodiments.

FIG. 14 is a flowchart showing a method of automatically determining a final position of a patient's dentition, according to an illustrative embodiment.

FIGS. 15A-15B show examples of a user approving a treatment plan, according to illustrative embodiments.

FIG. 16 is a flowchart showing a method of automatically verifying the safety and clinical efficiency of an orthodontic treatment plan by performing a clinical assessment on the treatment plan.

FIGS. 17A-17C are block diagrams depicting an example of a treatment plan interface architecture, according to illustrative embodiments.

FIGS. 18A-18B is block diagrams depicting an example of a treatment plan interface architecture, according to illustrative embodiments.

FIGS. 19A-19I are example illustrations depicting a treatment plan interface, according to illustrative embodiments.

FIGS. 20A-20C are example illustrations depicting a treatment plan interface, according to illustrative embodiments.

FIGS. 21A-21J are example illustrations depicting a treatment plan interface, according to illustrative embodiments.

FIGS. 22A-22B are flowcharts for methods of visualizing a treatment of teeth, according to illustrative embodiments.

FIGS. 23A-23G are example illustrations depicting a treatment plan interface, according to illustrative embodiments.

FIG. 24 is an example dental appliance, according to illustrative embodiments.

### DETAILED DESCRIPTION

The present disclosure is directed to systems and methods for automatically determining a final treatment plan position of a patient's dentition. According to various embodiments, the systems and methods described herein may include maintaining a geometric encoder model and a final position model. The final position model may be configured to determine movement of teeth (including translation movement and rotation movement) of a dentition from initial positions to final treatment planning positions. The final position model may be trained on a training set comprising a plurality of compressed three-dimensional (3D) training representations of dentitions comprising a plurality of teeth, and corresponding tooth movements to respective planned final positions post-treatment. The geometric encoder model may be configured to encode a 3D geometry representing surfaces of teeth into compressed representations. The geometric encoder models may be part of an autoencoder trained to encode 3D geometric information (such as point cloud distributions) into a compressed representation (such as a vector of floating point values). The systems and methods described herein may receive a first 3D representation of a dentition comprising a plurality of teeth of a patient in an initial position. The first 3D representation may include a plurality of tooth representations including a plurality of points representing surfaces of a respective tooth of the dentition. The systems and methods described herein may generate, for each tooth representation, a compressed tooth representation by transforming the initial 3D representation using the geometric encoder model. The systems and methods described herein may determine tooth movements of the plurality of teeth of the dentition from the initial position to a final position, responsive to applying each compressed tooth representation to the final position model. The systems and methods described herein may generate a second 3D representation of the dentition comprising the plurality of teeth of the patient in the final treatment planning position. Generating the second 3D representation may include applying the tooth movements to the initial 3D representation to move the orientation and rotation of each tooth (or group of teeth) into a final treatment plan position.

The systems and methods described herein may be trained based on previous (or historic) treatment plan data. The treatment plan data may be maintained or stored by a provider of the dental aligners. In some embodiments, the previous treatment plan data may be limited to treatment plans deemed successful (e.g., treatment plans which did not require a mid-course correction, treatment plans receiving positive patient feedback in a survey, etc.). The previous treatment plan data may include, for example, 3D data corresponding to an initial position of the previous patient's dentition, teeth movement data (e.g., translation and/or rotation movements from the initial position to a respective final position), and/or 3D data corresponding to the final position of the previous patient's dentition, etc.

By using previous or historic treatment plan data to train the machine learning models set forth herein, the systems and methods described herein may learn to leverage full and actual 3D geometries for training or learning treatment planning movements. Other solutions may involve identifying previous similar cases in a database or other data structure, which can be time/resource consuming to identify, and may be problematic where a similar case has not been treated before. Rather, by training based on previous or historic treatment plan data, the systems and methods described herein may be capable of identifying or learning teeth movements for any combination of teeth positions, which results in a more flexible final position model. Additionally, since the systems and methods herein rely on full and actual 3D geometries for training or learning treatment planning movements rather than hand-crafted geometric information (such as landmarks performed by a human as part of labeling or training), the systems and methods described herein may not discard important 3D data used for training. For example, where landmarking is performed in other solutions, such solutions may not be trained on full 3D data sets. Since the full 3D data sets are not used, the treatment plans generated from such solutions may be based on incomplete data. On the other hand, by relying on full 3D data sets as described herein, the systems and methods described herein may be trained on more complete data and therefore result in more accurate treatment plans.

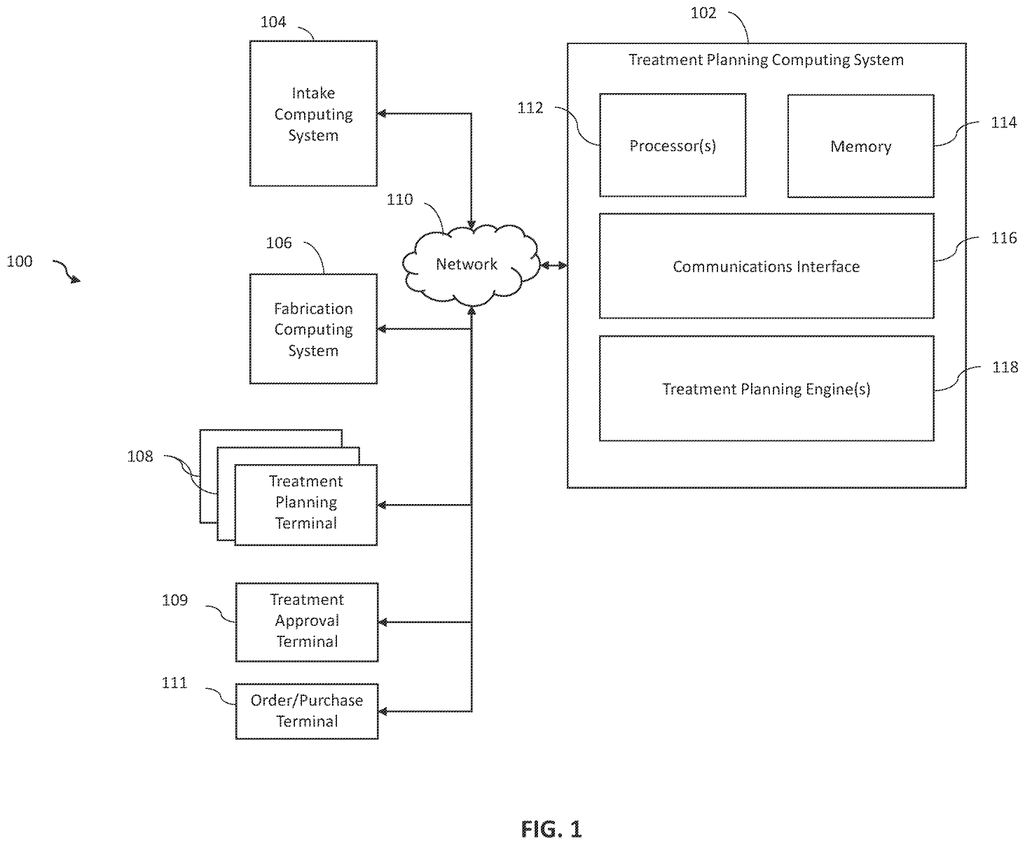

Referring to FIG. 1, a system 100 for orthodontic treatment is shown, according to an illustrative embodiment. As shown in FIG. 1, the system 100 includes a treatment planning computing system 102 communicably coupled to an intake computing system 104, a fabrication computing system 106, and one or more treatment planning terminals 108. In some embodiments, the treatment planning computing system 102 may be or may include one or more servers which are communicably coupled to a plurality of computing devices. In some embodiments, the treatment planning computing system 102 may include a plurality of servers, which may be located at a common location (e.g., a server bank) or may be distributed across a plurality of locations. The treatment planning computing system 102 may be communicably coupled to the intake computing system 104, fabrication computing system 106, treatment approval terminal 109, order/purchase terminal 111, and/or treatment planning terminals 108 via a communications link or network 110 (which may be or include various network connections configured to communicate, transmit, receive, or otherwise exchange data between addresses corresponding to the computing systems 102, 104, 106, 109, 111). The network 110 may be a Local Area Network (LAN), a Wide Area Network (WAN), a Wireless Local Area Network (WLAN), an Internet Area Network (IAN) or cloud-based network, etc. The network 110 may facilitate communication between the respective components of the system 100, as described in greater detail below.

The computing systems 102, 104, 106, 109, 111 include one or more processing circuits, which may include processor(s) 112 and memory 114. The processor(s) 112 may be a general purpose or specific purpose processor, an application specific integrated circuit (ASIC), one or more field programmable gate arrays (FPGAs), a group of processing components, or other suitable processing components. The processor(s) 112 may be configured to execute computer code or instructions stored in memory 114 or received from other computer readable media (e.g., CDROM, network storage, a remote server, etc.) to perform one or more of the processes described herein. The memory 114 may include one or more data storage devices (e.g., memory units, memory devices, computer-readable storage media, etc.) configured to store data, computer code, executable instructions, or other forms of computer-readable information. The memory 114 may include random access memory (RAM), read-only memory (ROM), hard drive storage, temporary storage, non-volatile memory, flash memory, optical memory, or any other suitable memory for storing software objects and/or computer instructions. The memory 114 may include database components, object code components, script components, or any other type of information structure for supporting the various activities and information structures described in the present disclosure. The memory 114 may be communicably connected to the processor 112 via the processing circuit, and may include computer code for executing (e.g., by processor(s) 112) one or more of the processes described herein.

The order/purchase terminal 111 may include any device(s), component(s), circuit(s), or other combination of hardware components designed or implemented to complete and/or guide a user in placing an order. An order may be a transaction that exchanges money from a patient for a product (e.g., an impression kit, dental aligners, etc.). The order/purchase terminal 111 may communicate with the fabrication computing system 106 and third party device (e.g., a patient or other user device) to guide a patient or other user through a payment/order completion system. In some embodiments, the order/purchase terminal 111 may communicate prompts to the user device to guide the user through the payment/order completion system. The prompts may include asking the patient for patient information (e.g., name, physical address, email address, phone number, credit card information) and product information (e.g., quantity of product, product name). In response to receiving information from the patient, the order/purchase terminal 111 initiates a product order. The initiated product order is transmitted to the fabrication computing system 106 to initiate the fabrication of one or more products (e.g., dental aligners). The initiated product order may also be transmitted to the intake computing system 104 to store/record the transaction and/or to initiate a product order from the computing system (e.g., a dental impression kit), and the like.

The treatment planning computing system 102 is shown to include a communications interface 116. The communications interface 116 can be or can include components configured to transmit and/or receive data from one or more remote sources (such as the computing devices, components, systems, and/or terminals described herein). In some embodiments, each of the servers, systems, terminals, and/or computing devices may include a respective communications interface 116 which permit exchange of data between the respective components of the system 100. As such, each of the respective communications interfaces 116 may permit or otherwise enable data to be exchanged between the respective computing systems 102, 104, 106, 109, 111. In some implementations, communications device(s) may access the network 110 to exchange data with various other communications device(s) via cellular access, a modem, broadband, Wi-Fi, satellite access, etc. via the communications interfaces 116.

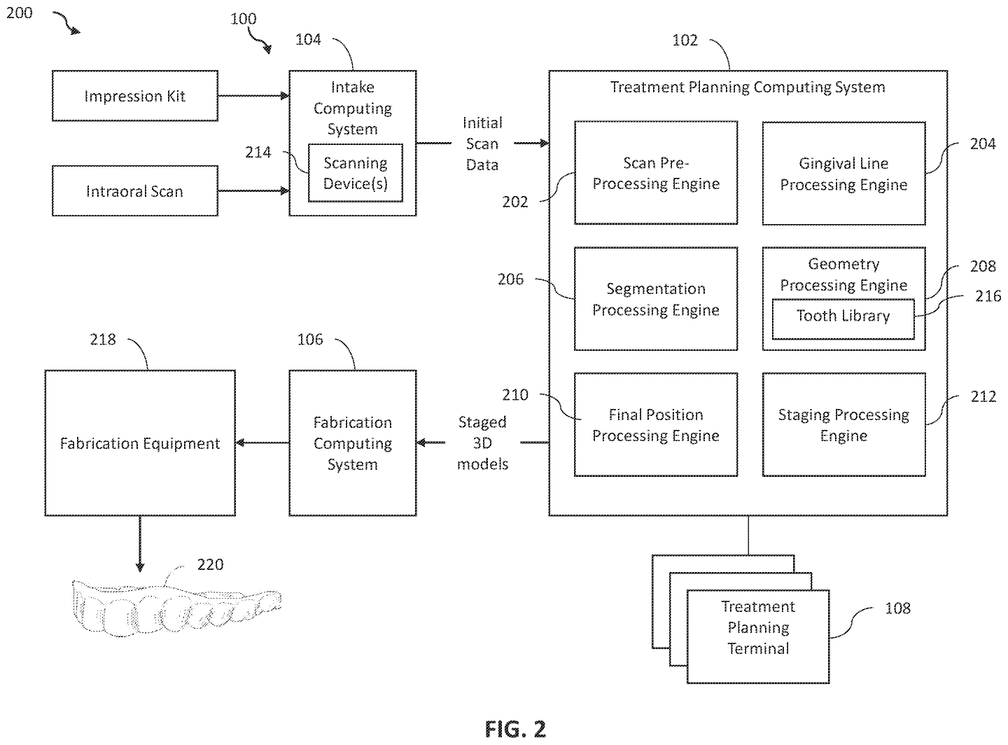

Referring now to FIG. 1 and FIG. 2, the treatment planning computing system 102 is shown to include one or more treatment planning engines 118. Specifically, FIG. 2 shows a treatment planning process flow 200 which may be implemented by the system 100 shown in FIG. 2, according to an illustrative embodiment. The treatment planning engine(s) 118 may be any device(s), component(s), circuit(s), or other combination of hardware components designed or implemented to receive inputs for and/or automatically generate a treatment plan from an initial three-dimensional (3D) model of a dentition. In some embodiments, the treatment planning engine(s) 118 may be instructions stored in memory 114 which are executable by the processor(s) 112. In some embodiments, the treatment planning engine(s) 118 may be stored at the treatment planning computing system 102 and accessible via a respective treatment planning terminal 108. As shown in FIG. 2, the treatment planning computing system 102 may include a scan pre-processing engine 202, a gingival line processing engine 204, a segmentation processing engine 206, a geometry processing engine 208, a final position processing engine 210, and a staging processing engine 212. While these engines 202-212 are shown in FIG. 2, it is noted that the system 100 may include any number of treatment planning engines 118, including additional engines which may be incorporated into, supplement, or replace one or more of the engines shown in FIG. 2.

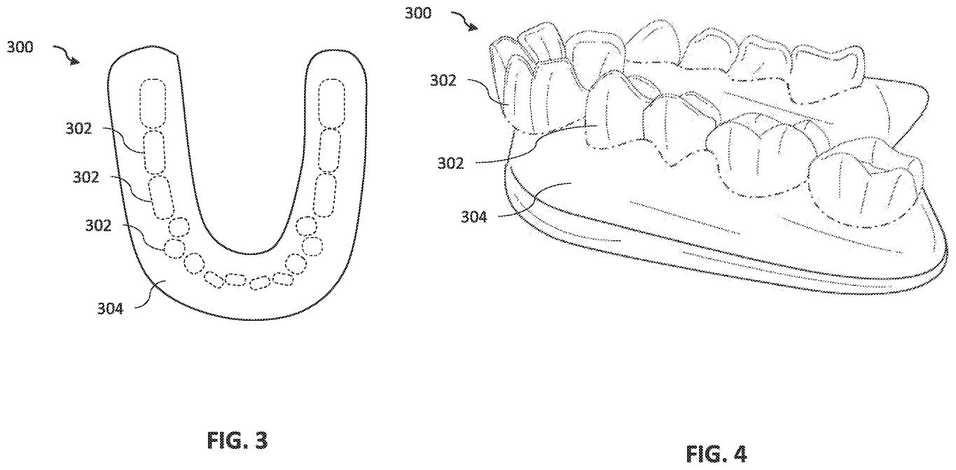

Referring to FIG. 2-FIG. 4, the intake computing system 104 may be configured to generate a 3D model of a dentition. Specifically, FIG. 3 and FIG. 4 show a simplified top-down view and a side perspective view of a 3D model of a dentition, respectively, according to illustrative embodiments. In some embodiments, the intake computing system 104 may be communicably coupled to or otherwise include one or more scanning devices 214. The intake computing system 104 may be communicably coupled to the scanning devices 214 via a wired or wireless connection. The scanning devices 214 may be or include any device, component, or hardware designed or implemented to generate, capture, or otherwise produce a 3D model 300 of an object, such as a dentition or dental arch. In some embodiments, the scanning devices 214 may include intraoral scanners configured to generate a 3D model of a dentition of a patient as the intraoral scanner passes over the dentition of the patient. For example, the intraoral scanner may be used during an intraoral scanning appointment, such as the intraoral scanning appointments described in U.S. Provisional Patent Appl. No. 62/660,141, titled “Arrangements for Intraoral Scanning,” filed Apr. 19, 2018, and U.S. patent application Ser. No. 16/130,762, titled “Arrangements for Intraoral Scanning,” filed Sep. 13, 2018. In some embodiments, the scanning devices 214 may include 3D scanners configured to scan a dental impression. The dental impression may be captured or administered by a patient using a dental impression kit similar to the dental impression kits described in U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, the contents of each of which are incorporated herein by reference in their entirety. In these and other embodiments, the scanning device(s) 214 may generally be configured to generate a 3D digital model of a dentition of a patient. As an example, the 3D digital model may be a point cloud representation of the dentition, a voxel representation, a spline representation, a mesh representation, or any other parametric model representation. In some embodiments, the scanning device(s) 214 may be configured to capture a two dimensional (2D) image of a dentition of the patient. The scanning device(s) 214 may be configured to generate a 3D digital model of the upper (i.e., maxillary) dentition and/or the lower (i.e., mandibular) dentition of the patient. The 3D digital model may include a digital representation of the patient's teeth 302 and/or gingiva 304. The scanning device(s) 214 may be configured to generate 3D digital models of the patient's dentition prior to treatment (i.e., with their teeth in an initial position). In some embodiments, the scanning device(s) 214 may be configured to generate the 3D digital models of the patient's dentition in real-time (e.g., as the dentition/impression) is scanned. In some embodiments, the scanning device(s) 214 may be configured to export, transmit, send, or otherwise provide data obtained during the scan to an external source which generates the 3D digital model, and transmits the 3D digital model to the intake computing system 104.

The intake computing system 104 may be configured to transmit, send, or otherwise provide the 3D digital model to the treatment planning computing system 102. In some embodiments, the intake computing system 104 may be configured to provide the 3D digital model of the patient's dentition to the treatment planning computing system 102 by uploading the 3D digital model to a patient file for the patient. The intake computing system 104 may be configured to provide the 3D digital model of the patient's upper and/or lower dentition at their initial (i.e., pre-treatment) position. The 3D digital model of the patient's upper and/or lower dentition may together form initial scan data which represents an initial position of the patient's teeth prior to treatment.

The treatment planning computing system 102 may be configured to receive the initial scan data from the intake computing system 104 (e.g., from the scanning device(s) 214 directly, indirectly via an external source following the scanning device(s) 214 providing data captured during the scan to the external source, etc.). As described in greater detail below, the treatment planning computing system 102 may include one or more treatment planning engines 118 configured or designed to generate a treatment plan based on or using the initial scan data.

Referring to FIG. 2, the treatment planning computing system 102 is shown to include a scan pre-processing engine 202. The scan pre-processing engine 202 may be or include any device(s), component(s), circuit(s), or other combination of hardware components designed or implemented to modify, correct, adjust, or otherwise process initial scan data received from the intake computing system 104 prior to generating a treatment plan. Generally, the scan pre-processing engine 202 may be configured to standardize and/or normalize the initial scan data such that subsequent processing (e.g., the final position processing engine) operates (and learns) on stable data (e.g., data that is not significantly varied with respect to number of points in a point cloud or other 3D representation, noise, smoothing artifacts, and the like). In some implementations, if a scan pre-processing engine 202 is employed to modify the initial scan data, a post-processing engine (not shown) may be employed to modify, correct, adjust, or otherwise process the output data. For example, if an input point cloud is upsampled during pre-processing, then the output point cloud may be downsampled during post-processing. The scan pre-processing engine 202 may be configured to process the initial scan data by applying one or more surface smoothing, resampling, and/or artifact removing algorithms to the initial scan data and/or 3D digital models. The scan pre-processing engine 202 may be configured to fill one or more holes or gaps in the 3D digital models. In some embodiments, the scan pre-processing engine 202 may be configured to receive inputs from a treatment planning terminal 108 to process the initial scan data. For example, the scan pre-processing engine 202 may be configured to receive inputs to smooth, refine, adjust, or otherwise process the initial scan data.

The inputs may include a selection of a smoothing processing tool presented on a user interface of the treatment planning terminal 108 showing the 3D digital model(s). As a user of the treatment planning terminal 108 selects various portions of the 3D digital model(s) using the smoothing processing tool, the scan pre-processing engine 202 may correspondingly smooth the 3D digital model at (and/or around) the selected portion. Similarly, the scan pre-processing engine 202 may be configured receive a selection of a gap filling processing tool presented on the user interface of the treatment planning terminal 108 to fill gaps in the 3D digital model(s).

In some embodiments, the scan pre-processing engine 202 may be configured to receive inputs for removing a portion of the gingiva represented in the 3D digital model of the dentition. For example, the scan pre-processing engine 202 may be configured to receive a selection (on a user interface of the treatment planning terminal 108) of a gingiva trimming tool which selectively removes gingival form the 3D digital model of the dentition. A user of the treatment planning terminal 108 may select a portion of the gingiva to remove using the gingiva trimming tool. The portion may be a lower portion of the gingiva represented in the digital model opposite the teeth. For example, where the 3D digital model shows a mandibular dentition, the portion of the gingiva removed from the 3D digital model may be the lower portion of the gingiva closest to the lower jaw. Similarly, where the 3D digital model shows a maxillary dentition, the portion of the gingiva removed from the 3D digital model may be the upper portion of the gingiva closest to the upper jaw.

The scan pre-processing engine 202 may also be configured to generate the 3D digital data given other representations of data (e.g., 2D images) and subsequently smooth/modify/trim the 3D digital data as described herein. For example, the 3D digital data may be obtained using 2D image reconstruction. In one embodiment, the scan pre-processing engine 202 may employ photogrammetry, for instance, to extract 3D measurements from captured 2D images (e.g., captured images from the scanning device 214). The scan pre-processing engine 202 may perform photogrammetry by comparing known measurements (e.g., known tooth measurements) with measurements of tooth features in the 2D image. The lengths/sizes of various features include tooth size measurements, tooth orientation measurements, and the like. Performing photogrammetry results in the determination of a position, orientation, size, and/or rotation of a tooth in the image. In some embodiments, the scan pre-processing engine 202 may perform photogrammetry using measurements of average teeth features from one or more databases (e.g., stored in memory 114). In some embodiments, the scan pre-processing engine 202 may receive particular measurements of a patient (e.g., entered into by a user at a treatment planning terminal 108 when the patient is present at the treatment planning terminal). The scan pre-processing engine 202 may compare the known measurements of teeth features with dimensions/measurements of the teeth features in the captured image to determine the position, orientation, size and/or rotation of the teeth features in the image.

Additionally or alternatively, the scan pre-processing engine 202 may use triangulation to generate a three-dimensional model of the patient based on images from various perspectives (e.g., multiple images captured). As an example, the scan pre-processing engine 202 may associate a two-dimensional pixel in the subsequent images with a ray in three-dimensional space. Given multiple perspectives of the image (e.g., at least two subsequent images capture the position of the patient from at least two different perspectives), the scan pre-processing engine 202 may determine a three-dimensional point from the intersection of at least two rays from pixels of the subsequent images.

The scan pre-processing engine 202 may execute various consistency functions to determine that the rays from the subsequent images are associated with consistent pixels. For instance, a pixel from a first perspective of an image, mapped to a three-dimensional point using a ray based on the first perspective of the image, is consistent with a pixel from a second perspective of an image mapped to the same three-dimensional point using a ray from the second perspective of the image. The scan pre-processing engine 202 may determine from the consistency functions, whether the pixels used to determine the three-dimensional point have similar colors, similar textures the textures, similar opacity, and the like.

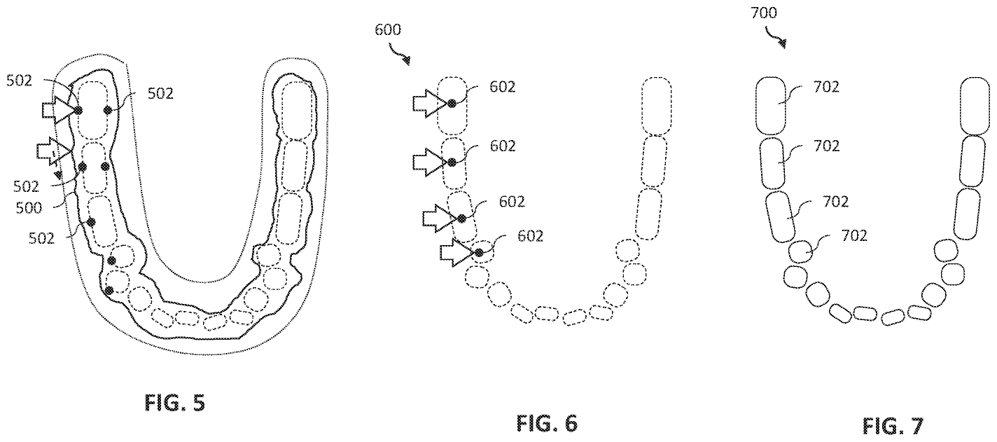

Referring now to FIG. 2 and FIG. 5, the treatment planning computing system 102 is shown to include a gingival line processing engine 204. Specifically, FIG. 5 shows a trace of a gingiva-tooth interface on the model 300 shown in FIG. 3 and FIG. 4. The gingival line processing engine 204 may be or include any device(s), component(s), circuit(s), or other combination of hardware components designed or implemented to determine, identify, or otherwise define a gingival line of the 3D digital models. The gingival line may be or include the interface between the gingiva and teeth represented in the 3D digital models. In some embodiments, the gingival line processing engine 204 may be configured to receive inputs from the treatment planning terminal 108 for defining the gingival line. The treatment planning terminal 108 may show a gingival line defining tool on a user interface which includes the 3D digital models.

The gingival line defining tool may be used for defining or otherwise determining the gingival line for the 3D digital models. As one example, the gingival line defining tool may be used to trace a rough gingival line 500. For example, a user of the treatment planning terminal 108 may select the gingival line defining tool on the user interface and drag the gingival line defining tool along an approximate gingival line of the 3D digital model. As another example, the gingival line defining tool may be used to select (e.g., on the user interface shown on the treatment planning terminal 108) lowest points 502 at the teeth-gingiva interface for each of the teeth in the 3D digital model.

The gingival line processing engine 204 may be configured to receive the inputs provided by the user via the gingival line defining tool on the user interface of the treatment planning terminal 108 for generating or otherwise defining the gingival line. In some embodiments, the gingival line processing engine 204 may be configured to use the inputs to identify a surface transition on or near the selected inputs. For example, where the input selects a lowest point 502 (or a portion of the rough gingival line 500 near the lowest point 502) on a respective tooth, the gingival line processing engine 204 may identify a surface transition or seam at or near the lowest point 502 which is at the gingival margin. The gingival line processing engine 204 may define the transition or seam as the gingival line. In some embodiments, the gingival line processing engine 204 may automatically determine the gingival line (e.g., without receiving inputs from the treatment planning terminal 108) by segmenting each tooth (a portion of teeth, a group of teeth) to determine the teeth-gingiva interface. Accordingly, the gingival line processing engine 204 may be configured to differentiate teeth and gingiva in the 3D digital model via one or more image processing algorithms and/or machine learning algorithms trained to differentiate teeth from gingiva). The gingival line processing engine 204 may define the gingival line for each of the teeth 302 (or a portion/group of teeth) included in the 3D digital model 300 (or a 2D image). The gingival line processing engine 204 may be configured to generate a tooth model using the gingival line of the teeth 302 in the 3D digital model 300. The gingival line processing engine 204 may be configured to generate the tooth model by separating the 3D digital model along the gingival line. The tooth model may be the portion of the 3D digital model which is separated along the gingival line and includes digital representations of the patient's teeth.

Referring now to FIG. 2 and FIG. 6, the treatment planning computing system 102 is shown to include a segmentation processing engine 206. Specifically, FIG. 6 shows a view of the tooth model 600 generated by the gingival line processing engine 204. The segmentation processing engine 206 may be or include any device(s), component(s), circuit(s), or other combination of hardware components designed or implemented to determine, identify, or otherwise segment individual teeth from the tooth model. For example, the segmentation processing engine 206 may be configured to differentiate teeth in the tooth model 600 via one or more image processing algorithms and/or machine learning algorithms trained to differentiate teeth. In some embodiments, the segmentation processing engine 206 may be configured to receive inputs (e.g., via a user interface shown on the treatment planning terminal 108) which select the teeth (e.g., points 602 on the teeth) in the tooth model 600. For example, the user interface may include a segmentation tool which, when selected, allows a user to select points 602 on each of the individual teeth in the tooth model 600. In some embodiments, the selection of each teeth may also assign a label to the teeth. The label may include tooth numbers (e.g., according to FDI world dental federation notation, the universal numbering system, Palmer notation, etc.) for each of the teeth in the tooth model 600. As shown in FIG. 6, the user may select individual teeth in the tooth model 600 to assign a label to the teeth.

Referring now to FIG. 7, depicted is a segmented tooth model 700 generated from the tooth model 600 shown in FIG. 6. The segmentation processing engine 206 may be configured to receive the selection of the teeth from the user via the user interface of the treatment planning terminal 108. The segmentation processing engine 206 may be configured to separate each of the teeth selected by the user on the user interface. For example, the segmentation processing engine 206 may be configured to identify or determine a gap between two adjacent points 602. The segmentation processing engine 206 may be configured to use the gap as a boundary defining or separating two teeth. The segmentation processing engine 206 may be configured to define boundaries for each of the teeth in the tooth model 600. The segmentation processing engine 206 may be configured to generate the segmented tooth model 700 including segmented teeth 702 using the defined boundaries generated from the selection of the points 602 on the teeth in the tooth model 600.

The treatment planning computing system 102 is shown to include a geometry processing engine 208. The geometry processing engine 208 may be or include any device(s), component(s), circuit(s), or other combination of hardware components designed or implemented to determine, identify, or otherwise generate whole tooth models for each of the teeth in the 3D digital model. Once the segmentation processing engine 206 generates the segmented tooth model 700, the geometry processing engine 208 may be configured to use the segmented teeth to generate a whole tooth model for each of the segmented teeth. Since the teeth have been separated along the gingival line by the gingival line processing engine 204 (as described above with reference to FIG. 6), the segmented teeth may only include crowns (e.g., the segmented teeth may not include any roots). The gingival line processing engine 204 may be configured to generate a whole tooth model including both crown and roots using the segmented teeth. In some embodiments, the segmentation processing engine 206 may be configured to generate the whole tooth models using the labels assigned to each of the teeth in the segmented tooth model 700. For example, the geometry processing engine 208 may be configured to access a tooth library 216. The tooth library 216 may include a library or database having a plurality of whole tooth models. The plurality of whole tooth models may include tooth models for each of the types of teeth in a dentition. The plurality of whole tooth models may be labeled or grouped according to tooth numbers.

The geometry processing engine 208 may be configured to generate the whole tooth models for a segmented tooth by performing a look-up function in the tooth library 216 using the label assigned to the segmented tooth to identify a corresponding whole tooth model. The geometry processing engine 208 may be configured to morph the whole tooth model identified in the tooth library 216 to correspond to the shape (e.g., surface contours) of the segmented tooth. In some embodiments, the geometry processing engine 208 may be configured to generate the whole tooth model by stitching the morphed whole tooth model from the tooth library 216 to the segmented tooth, such that the whole tooth model includes a portion (e.g., a root portion) from the tooth library 216 and a portion (e.g., a crown portion) from the segmented tooth. In some embodiments, the geometry processing engine 208 may be configured to generate the whole tooth model by replacing the segmented tooth with the morphed tooth model from the tooth library. In these and other embodiments, the geometry processing engine 208 may be configured to generate whole tooth models, including both crown and roots, for each of the teeth in a 3D digital model. The whole tooth models of each of the teeth in the 3D digital model may depict, show, or otherwise represent an initial position of the patient's dentition.

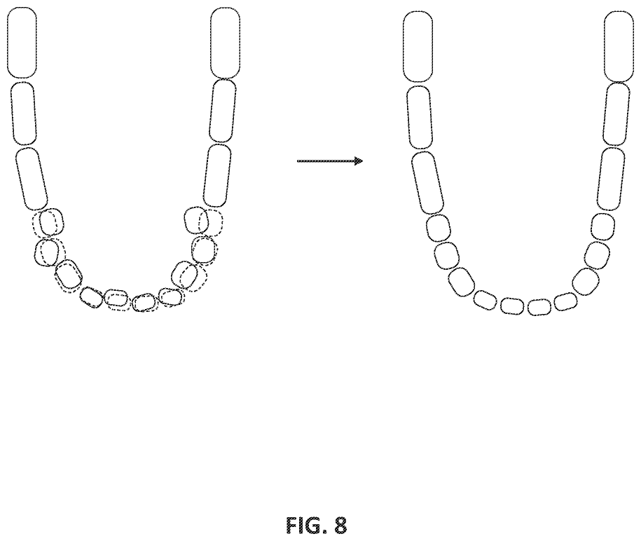

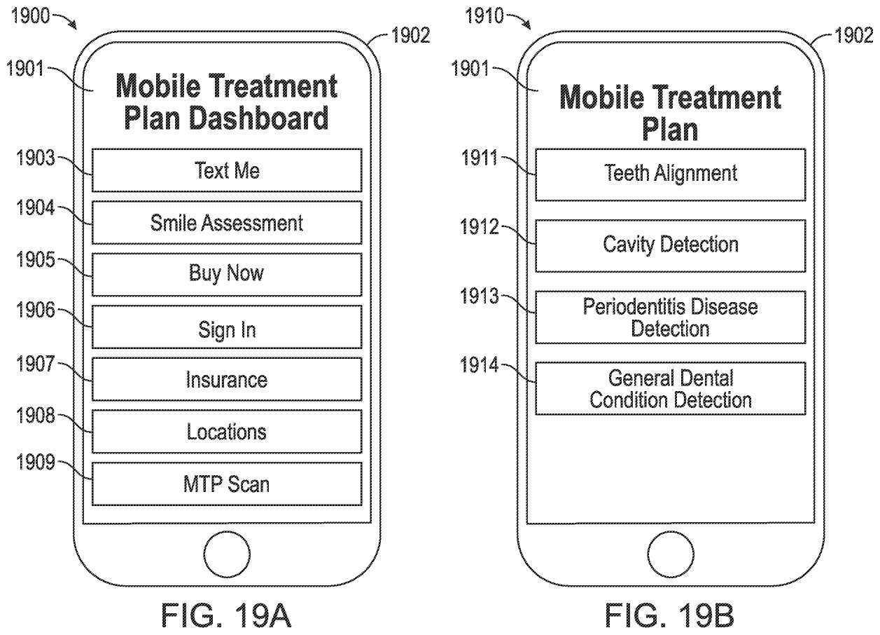

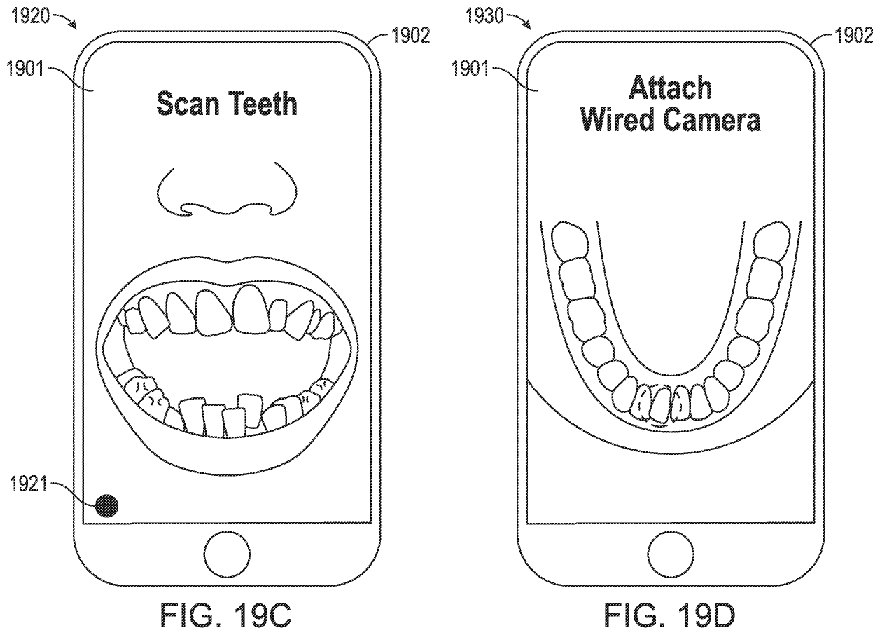

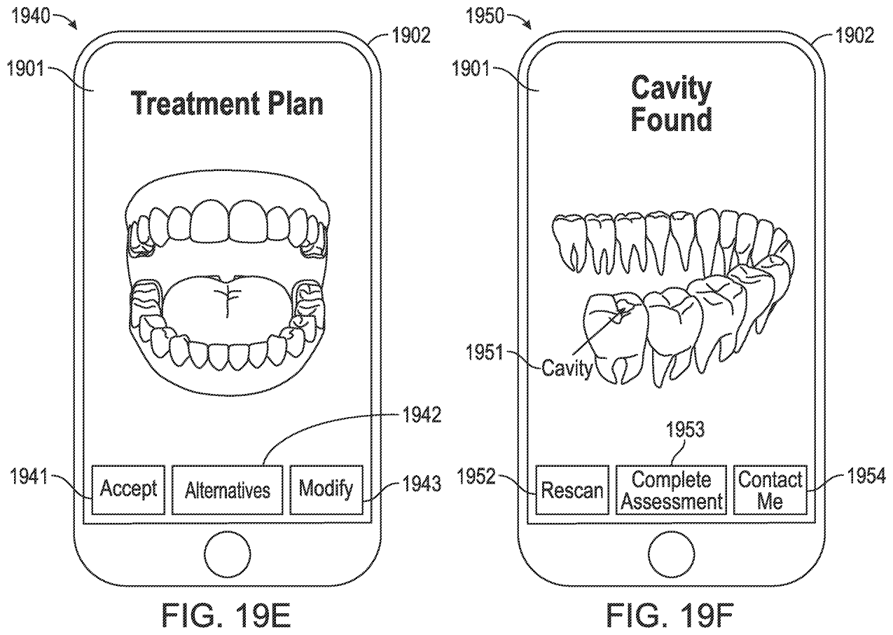

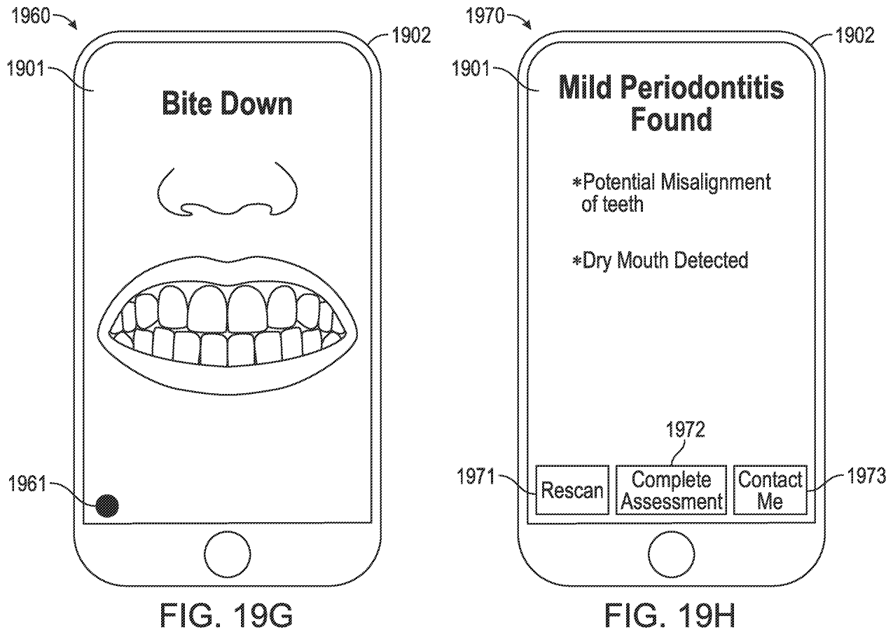

Referring now to FIG. 2 and FIG. 8, the treatment planning computing system 102 is shown to include a final position processing engine 210. FIG. 8 shows one example of a target final position of the dentition from the initial position of the dentition shown in FIG. 7 from a top-down view (depicted in FIGS. 19-21 and 23 as a mobile treatment plan). The final position processing engine 210 may be or may include any device(s), component(s), circuit(s), or other combination of hardware components designed or implemented to determine, identify, or otherwise generate (or determine) a final position of the patient's teeth. The final position processing engine 210 may be configured to generate the treatment plan by manipulating individual 3D models of teeth within the 3D model (e.g., shown in FIG. 7). In some embodiments, the final position processing engine 210 may be configured to receive inputs for generating the final position of the patient's teeth. The final position may be a target position of the teeth post-orthodontic treatment or at a last stage of realignment. A user of the treatment planning terminal 108 may provide one or more inputs for each tooth or a subset of the teeth in the initial 3D model to move the teeth from their initial position to their final position (shown in dot-dash). For example, the treatment planning terminal 108 may be configured to receive inputs to drag, shift, rotate, or otherwise move individual teeth to their final position, incrementally shift the teeth to their final position, etc. The movements may include lateral/longitudinal movements, rotation movements, translational movements, etc. The movements may include intrusions and/or extrusions of the teeth relative to the occlusal axis, as will be described below.

In some embodiments, the manipulation of the 3D model may show a final (or target) position of the teeth of the patient following orthodontic treatment or at a last stage of realignment via dental aligners. In some embodiments, the final position processing engine 210 may be configured to apply one or more movement thresholds (e.g., a maximum lateral and/or rotation movement for treatment) to each of the individual 3D teeth models for generating the final position. As such, the final position may be generated in accordance with the movement thresholds.

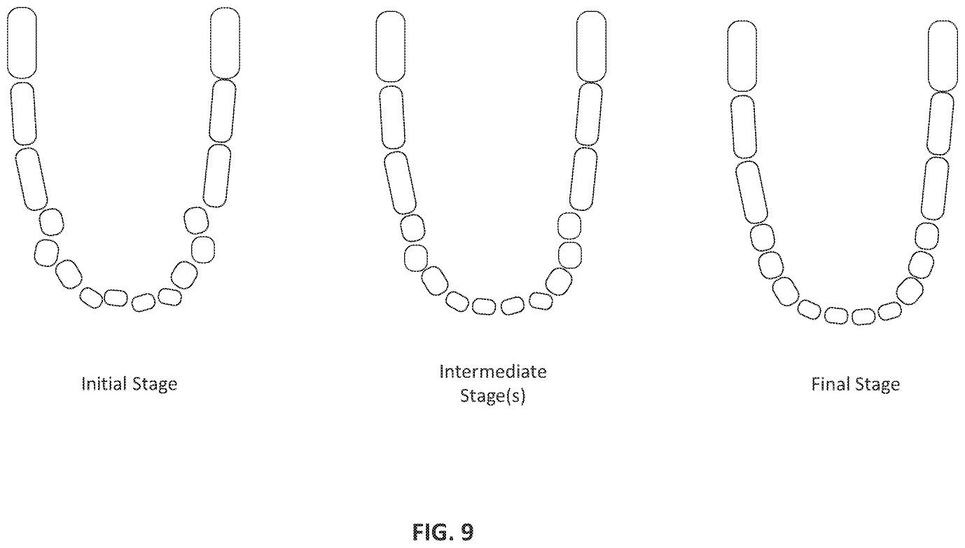

Referring now to FIG. 2 and FIG. 9, the treatment planning computing system 102 is shown to include a staging processing engine 212. Specifically, FIG. 9 shows a series of stages of the dentition from the initial position shown in FIG. 7 to the target final position shown in FIG. 8, according to an illustrative embodiment. The staging processing engine 212 may be or include any device(s), component(s), circuit(s), or other combination of hardware components designed or implemented to determine, identify, or otherwise generate stages of treatment (e.g., a treatment plan) from the initial position to the final position of the patient's teeth. In some embodiments, the staging processing engine 212 may be configured to receive inputs (e.g., via a user interface of the treatment planning terminal 108) for generating the stages. In some embodiments, the staging processing engine 212 may be configured to automatically compute or determine the stages based on the movements from the initial to the final position. The staging processing engine 212 may be configured to apply one or more movement thresholds (e.g., a maximum lateral and/or rotation movement for a respective stage) to each stage of treatment plan. The staging processing engine 212 may be configured to generate the stages as 3D digital models of the patient's teeth as they progress from their initial position to their final position. For example, and as shown in FIG. 9, the stages may include an initial stage including a 3D digital model of the patient's teeth at their initial position, one or more intermediate stages including 3D digital model(s) of the patient's teeth at one or more intermediate positions, and a final stage including a 3D digital model of the patient's teeth at the final position.

In some embodiments, the staging processing engine 212 may be configured to generate at least one intermediate stage for each tooth based on a difference between the initial position of the tooth and the final position of the tooth. For instance, where the staging processing engine 212 generates one intermediate stage, the intermediate stage may be a halfway point between the initial position of the tooth and the final position of the tooth. Each of the stages may together form a treatment plan for the patient, and may include a series or set of 3D digital models.

Following generating the stages, the treatment planning computing system 102 may be configured to transmit, send, or otherwise provide the staged 3D digital models to the fabrication computing system 106. In some embodiments, the treatment planning computing system 102 may be configured to provide the staged 3D digital models to the fabrication computing system 106 by uploading the staged 3D digital models to a patient file which is accessible via the fabrication computing system 106. In some embodiments, the treatment planning computing system 102 may be configured to provide the staged 3D digital models to the fabrication computing system 106 by sending the staged 3D digital models to an address (e.g., an email address, IP address, etc.) for the fabrication computing system 106.

The fabrication computing system 106 can include a fabrication computing device and fabrication equipment 218 configured to produce, manufacture, or otherwise fabricate dental aligners. The fabrication computing system 106 may be configured to receive a plurality of staged 3D digital models corresponding to the treatment plan for the patient. As stated above, each 3D digital model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

The fabrication computing system 106 may be configured to send the staged 3D models to fabrication equipment 218 for generating, constructing, building, or otherwise producing dental aligners 220. In some embodiments, the fabrication equipment 218 may include a 3D printing system. The 3D printing system may be used to 3D print physical models corresponding the 3D models of the treatment plan. As such, the 3D printing system may be configured to fabricate physical models which represent each stage of the treatment plan. In some implementations, the fabrication equipment 218 may include casting equipment configured to cast, etch, or otherwise generate physical models based on the 3D models of the treatment plan. Where the 3D printing system generates physical models, the fabrication equipment 218 may also include a thermoforming system. The thermoforming system may be configured to thermoform a polymeric material to the physical models, and cut, trim, or otherwise remove excess polymeric material from the physical models to fabricate a dental aligner. In some embodiments, the 3D printing system may be configured to directly fabricate dental aligners 220 (e.g., by 3D printing the dental aligners 220 directly based on the 3D models of the treatment plan). Additional details corresponding to fabricating dental aligners 220 are described in U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, and U.S. Pat. No. 10,315,353, titled “Systems and Methods for Thermoforming Dental Aligners,” filed Nov. 13, 2018, the contents of each of which are incorporated herein by reference in their entirety.

The fabrication equipment 218 may be configured to generate or otherwise fabricate dental aligners 220 for each stage of the treatment plan. In some instances, each stage may include a plurality of dental aligners 220 (e.g., a plurality of dental aligners 220 for the first stage of the treatment plan, a plurality of dental aligners 220 for the intermediate stage(s) of the treatment plan, a plurality of dental aligners 220 for the final stage of the treatment plan, etc.). Each of the dental aligners 220 may be worn by the patient in a particular sequence for a predetermined duration (e.g., two weeks for a first dental aligner 220 of the first stage, one week for a second dental aligner 220 of the first stage, etc.).

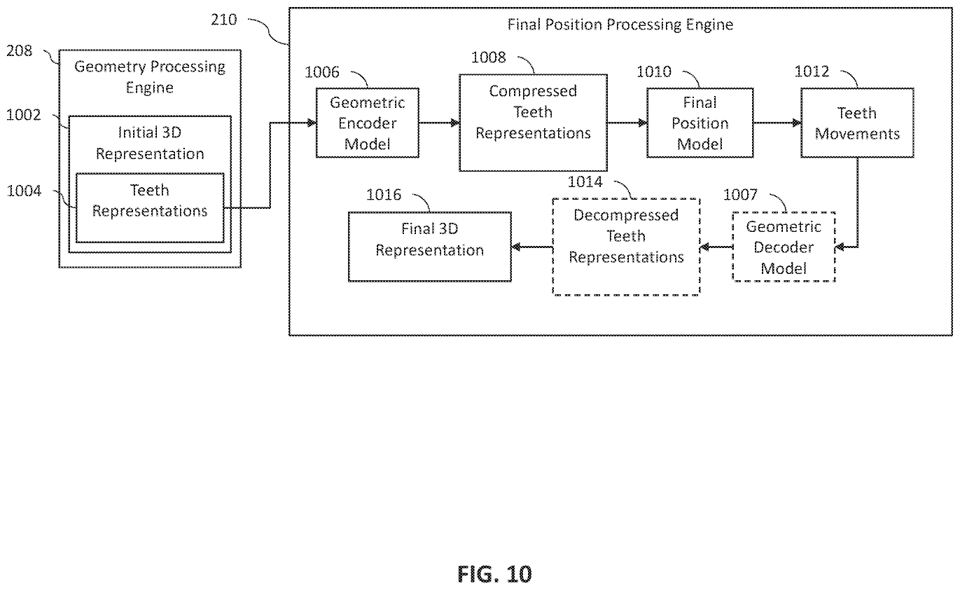

Referring now to FIG. 10, depicted is a view of the final position processing engine 210 of the treatment planning computing system 102 of FIG. 2, according to an illustrative embodiment. As described in greater detail below, the final position processing engine 210 may be configured to automatically determine, derive, or otherwise generate a three-dimensional (3D) representation of a final position of a dentition (also referred to herein as a final 3D representation). The final position processing engine 210 may be configured to generate the final 3D representation responsive to a user selection on the treatment planning terminal 108. The final position processing engine 210 may be configured to generate the final 3D representation by applying an initial 3D representation (e.g., generated by the geometry processing engine 208 as described above) to one or more machine learning models.

The final position processing engine 210 may be configured to receive an initial 3D representation 1002 of a patient's dentition. The final position processing engine 210 may be configured to receive the initial 3D representation 1002 from the geometry processing engine 208. In some embodiments, before the final position processing engine 210 receives initial 3D representation 1002 of the patient's detention, the scan pre-processing engine 202 may normalize and/or standardize the initial 3D representation 1002. For example, the scan pre-processing engine 202 may apply one or more surface smoothing, resampling, and/or artifact removing algorithms to the initial 3D representation 1002. In some embodiments, the initial 3D representation 1002 may be or include a point cloud including points located on surfaces of the patient's dentition. In some embodiments, the initial 3D representation 1002 may be mesh representation, a voxel representation, a spline representation, or any other parametric representation. The initial 3D representation 1002 may include teeth representations 1004 representing each of the teeth (or a group of teeth) in the patient's dentition. Each tooth representation 1004 may include a point cloud including points located on surfaces of the respective tooth. Each of the tooth representations 1004 may together form the initial 3D representation 1002 of the patient's dentition.

The final position processing engine 210 may include, maintain, or otherwise access a geometric encoder model 1006 and, in some cases, a geometric decoder model 1007. The geometric encoder model 1006 may be or include any device, component, or other hardware designed or implemented to convert a tooth representation into a latent space representation (such as a vector), and back into a tooth representation.

The geometric encoder model 1006 may include an encoder. In some embodiments, the geometric encoder model 1006 may be configured to convert, transform, or otherwise generate a vector representation of a 3D representation (such as a point cloud or mesh). The geometric encoder model 1006 may encode and compress the geometry of teeth representations 1004 using one or more deep learning algorithms such as PointNet or PointNet++. The geometric encoder model 1006 may be an encoder of an autoencoder. Accordingly, the geometric encoder model 1006 may be trained using self-supervised learning (or unsupervised learning) based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation).

In the above example, if the geometric encoder model 1006 is an autoencoder, an encoder portion of the autoencoder may learn the latent space representation of one or more teeth in the teeth representation 1004 (e.g., compressing the tooth, encoding the full 3D geometry of each tooth). For example, a convolutional autoencoder may employ convolutional layer(s) and pooling layer(s) to downsample the teeth in the teeth representation 1004 to determine the latent space representation of the teeth in the teeth representation. The convolutional layer(s) convolve the one or more teeth in the teeth representation 1004 with one or more filters to extract features of the teeth representation 1004 to create a feature map. The filters, commonly known as kernels, are of arbitrary sizes and define the field of view for the convolution such that the dimensionality reduces. The pooling layer(s) may further downsample the data by applying a pooling window to the feature map. The pooling layer may be a max pooling layer (or any other type of pooling later) that detects prominent features. In some configurations, the pooling layer may be an average pooling layer. The pooling layer(s) reduce the dimensionality of the feature map to further downsample the feature map.

The latent space representation may include a vector representation of each tooth. The vector representation may be or include a numerical representation of each tooth. The geometric encoder model 1006 may be configured to generate a tensor which combines each vector representation. The tensor may correspond to the number of teeth in a dental arch and the latent space representation for each tooth in the dental arch. For example, a dental arch includes 16 teeth per arch. The geometric encoder model 1006 may be configured to generate a tensor of 16×N, where 16 is a numerical representation of each tooth representation, and N is the number of points in a vector representation of the tooth. Where a dental arch of a patient has a missing tooth, the corresponding tensor generated by the geometric encoder model 1006 may have null or zero values assigned to the corresponding tooth representation in the tensor.

A decoder portion of the autoencoder (e.g., the geometric decoder model 1007) may decompress (or reconstruct) the encoded full 3D geometry of each tooth. The autoencoder operates using the encoder (e.g., geometric encoder model 1006) and decoder (e.g., geometric decoder model 1007) and compares the teeth in the teeth representation 1004 to the decompressed tooth representations following compression, encoding, and subsequent decompression to learn how to better encode the full 3D geometry of each tooth. For example, the target value (e.g., the decompressed tooth representations following compression, encoding, and subsequent decompression) is set to equal the input (e.g., the tooth representation 1004). Accordingly, an example loss function that trains the geometric encoder model 1006 and geometric decoder model 1007 may be based on the 3D error of the reconstructed full 3D geometry of each tooth (or groups of teeth). In some embodiments, the geometric encoder model 1006 may be trained to generate compressed teeth representations of teeth crowns. For example, the target value (e.g., the decompressed tooth representations including the tooth crown following compression, encoding, and subsequent decompression) is set to equal the input (e.g., the tooth representation 1004 including the tooth crown). In some embodiments, the geometric encoder model 1006 may be trained to generate compressed teeth representations of teeth crowns and roots. For example, the target value (e.g., the decompressed tooth representations including the tooth crown and/or roots following compression, encoding, and subsequent decompression) is set to equal the input (e.g., the tooth representation 1004 including the tooth crown and/or roots). The crown or crown and root representations may be similar to the estimated teeth representations described above in connection with the geometry processing engine 208.

The final position processing engine 210 may train the geometric encoder model 1006 and geometric decoder model 1007 until a number of training iterations satisfies a threshold, the error between the decompressed tooth representations following compression, encoding, and subsequent decompression and one or more teeth in the teeth representation 1004 satisfies a threshold, and the like. Training the geometric encoder model 1006 represents the geometric encoder model 1006 encoding the 3D geometry of each tooth into a latent space representation (such as a vector). Accordingly, the compressed teeth representation 1008 (encoded, latent space representation, vector representation) full 3D geometry of each tooth (or a groups of teeth) may be substituted for the full 3D geometry of each tooth (or the groups of teeth) of the teeth representation 1004. In some embodiments, one or more additional vectors may be appended to the compressed teeth representation 1008. For example, the final position processing engine 210 may append a vector including the center of each tooth in 3D coordinates to the compressed teeth representation 1008 output from the geometric encoder model 1006. The final position processing engine 210 may determine the center of each tooth using a global coordinate system, or other coordinate systems.

Once the geometric encoder model 1006 is trained to compress tooth representations as described herein, the geometric encoder model 1006 may be deployed or otherwise used by the final position processing engine 210. Specifically, the final position processing engine 210 may be configured to apply the teeth representations 1004 from an initial 3D representation 1002 to the geometric encoder model 1006 to generate compressed teeth representations 1008. The geometric encoder model 1006 may be configured to generate compressed teeth representations 1008 from the teeth representations 1004 for each of the teeth representations 1004 and/or for a group of teeth.

Once the geometric encoder model 1006 generates a compressed tensor corresponding to the patient's dental arch, (e.g., each of the teeth and/or groups of teeth have been encoded via the geometric encoder model 1006 to compressed teeth representations 1008), the final position processing engine 210 may be configured to apply the tensor to a final position model 1010. The final position model 1010 may be or include any device, component, or other hardware designed or implemented to generate, identify, or otherwise determine tooth movements for each tooth of a dentition from initial positions to final positions. In some embodiments, the final position model 1010 may be configured to determine the final position of the teeth using a trained neural network. The neural network may be trained using supervised learning.

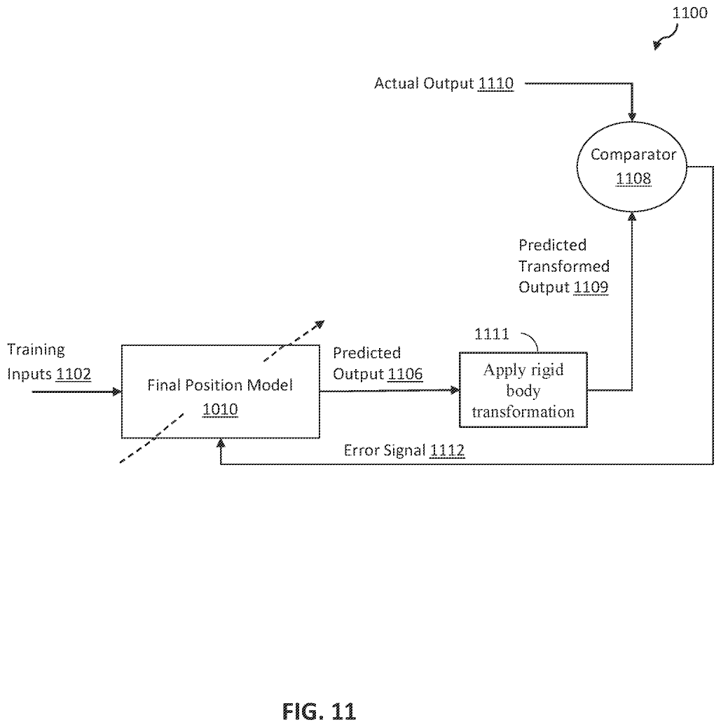

Referring to FIG. 11, a block diagram of an example system 1100 using supervised learning that may be used to determine a movement of the teeth (e.g., the final position of one or more of the patient's teeth post treatment described with respect to the final tooth orientation and translation) is shown according to an example embodiment. Supervised learning is a method of training a machine learning model given input-output pairs. An input-output pair is an input with an associated known output (e.g., an expected output). The final position model 1010 may be trained on known input-output pairs (e.g., full 3D geometric encoded and compressed representations of initial teeth positions and final teeth positions) such that the final position model 1010 can learn how to predict known outputs given known inputs. Once a final position model 1010 has learned how to predict known input-output pairs, the final position model 1010 can operate on unknown inputs to predict an output.

To train the final position model 1010 using supervised learning, training inputs 1102 and actual outputs 1110 may be provided to the final position model 1010. In some embodiments, training inputs 1102 may include a full 3D geometric encoded and compressed representation of each tooth (or a representation of a plurality of teeth) at an initial position. In some embodiments, training inputs 1102 may include encoded projected teeth representation(s) such that each tooth (or groups of teeth) in 3D is converted to an encoded 2D image configured to convey the shape (including the translation and orientation) and location of each tooth (or groups of teeth). Actual outputs 1110 may include a final position of each tooth (or a final position of groups of teeth after the teeth have undergone a treatment plan. The final position of each tooth (or groups of teeth) after the teeth have undergone the treatment plan may be in the form of a 3D representation (e.g., a point cloud) or an encoded latent space representation of each tooth (or groups of teeth).

The inputs 1102 and actual outputs 1110 may be stored in memory or other data structure accessible by the final position processing engine 210. The inputs 1102 and actual outputs 1110 may be received from a historic treatment plan or 3D tooth representation data from a data repository. In some embodiments, the historic treatment plan data may be limited to treatment plans deemed successful (e.g., treatment plans which did not require a mid-course correction, treatment plans receiving positive patient feedback in a survey, etc.). The historic treatment plan data may include, for example, 3D data corresponding to an initial position of the previous patient's dentition, teeth movement data (e.g., movements from the initial position to a respective final position), 3D data corresponding to the final position of the previous patient's dentition (which may be rotation components and translation components obtained from the treatment plan or from a post-treatment impression or intraoral scan), etc.

In an example, the inputs 1102 may be mesh representations of one or more teeth at an initial position. The actual outputs 1110 may be mesh representations of one or more teeth at a final position post treatment. The mesh representations of the teeth at the final position may be received by the final position processing engine 210 by receiving scans of the patient's teeth from the scanning device (e.g., scanning device 214 in FIG. 2) and generating a mesh representation. The mesh representations of the teeth at the final position may also be received by the final position processing engine 210 from a treating dentist (e.g., via a treatment planning terminal 108 in FIG. 1) as a digital representation of an anticipated final position post treatment.

The system 1100 is shown to include a comparator 1108. The comparator 1108 may be configured to compare the transformed predicted output 1109 to the actual output 1110. In some embodiments, the predicted output 1106 may be an encoded latent space representation of teeth at a predicted final position post treatment. For example, the predicted output 1106 may be a M×6 tensor where M represents the number of teeth (e.g., the same number of teeth that was input into the final position model 1010) and 6 represents the orientation and rotation at the final position post treatment (e.g., three translation components and three rotation components). The tensor becomes the rigid body transformation 1111 that, when applied to the initial position of each tooth (e.g., a 3D representation of one or more teeth at an initial position pre-treatment), moves the tooth to a predicted final position post treatment (e.g., the predicted transformed output 1109). In alternate embodiments, the rigid body transformation 1111 may be applied to the initial position of each tooth (e.g., training inputs 1102). In these embodiments, a decoder may decode the transformed decompressed latent space representation into a 3D representation.

The comparator 1108 is configured to compare an error of the 3D representation corresponding to the transformed predicted output 1109 and the 3D representation corresponding to the actual output 1110. In this manner, the loss is determined based on the final treatment plan geometry, not based on the treatment plan movements (e.g., a geometric accuracy assessment, a geometry based final positioning assessment, a geometric feature comparison). Accordingly, center of rotation and other axes are less important compared to the actual final geometry of each tooth (or groups of teeth) because the final geometry of each tooth includes final position rotation and translation components. The comparator 1108 may calculate the loss of the 3D representations using a 3D chamfer distance.

In some embodiments, the comparator 1108 may be configured to compare an actual encoded latent space representation (e.g., actual output 1110) to the predicted compressed transformed output (e.g., predicted transformed output 1109). In these embodiments, the actual encoded latent space representation may be determined by the final position processing engine 210 encoding an actual 3D representation of teeth at a final position post treatment using the trained geometric encoder model 1006. The predicted compressed transformed output may be determined by the final position processing engine 210 applying the rigid body transformation 1111 to the initial position of each tooth (e.g., training inputs 1102). In this manner, the loss is determined based on the final teeth movements (e.g., the translation/rotation components).

During training, the error (represented by error signal 1112) determined by the comparator 1108 may be used to adjust the weights in the final position model 1010 such that the final position model 1010 changes (or learns) over time to generate a relatively accurate prediction of final position of one or more teeth (and/or translation/rotation components of the final position of one or more teeth), using the input-output pairs. The final position model 1010 may be trained using the backpropagation algorithm, for instance. The backpropagation algorithm operates by propagating the error signal 1112. The error signal 1112 may be calculated each iteration (e.g., each pair of training inputs 1102 and associated actual outputs 1110), batch, and/or epoch and propagated through all of the algorithmic weights in the final position model 1010 such that the algorithmic weights adapt based on the amount of error. The error is minimized using a loss function. Non-limiting examples of loss functions may include the square error function, the room mean square error function, and/or the cross entropy error function.

The weighting coefficients of the final position model 1010 may be tuned to reduce the amount of error thereby minimizing the differences between (or otherwise converging) the predicted output 1106 and the actual output 1110. For instance, because the final position model 1010 is being trained to predict the final post treatment teeth position given the initial teeth position, the 3D representation of the predicted final teeth position (e.g., the predicted transformed output 1109) will iteratively converge to the actual final teeth position (e.g., an actual 3D representation of teeth post treatment). The final position processing engine 210 may train the geometric encoder model 1006 until the error determined at the comparator 1108 is within a certain threshold (or a threshold number of batches, epochs, or iterations have been reached). The final position model 1010 and associated weighting coefficients may subsequently be stored in memory or other data repository (e.g., a database) such that the trained final position model 1010 may be employed on unknown data (e.g., not training inputs 1102). Once trained and validated, the final position model 1010 may be employed during testing. During testing, the final position model 1010 may ingest unknown data to predict final teeth positions (e.g., a final 3D representations of one or more teeth, translation/rotation movement components of one or more teeth at a final position after treatment). For example, during testing, the final position model 1010 may ingest tooth position data (in a compressed representation, e.g., compressed teeth representation 1008) to the trained final position model 1010 to predict the final teeth movements (including the translation and rotation components). In a particular example, an upper arch 3D encoded latent space representation of 16×N+3 may be input to the final position model 1010, where 16 represents the 16 individual teeth in an upper arch, N represents the number of points in a vector representation, and 3 represents the position of the center of the tooth in 3D coordinates. The final position model 1010 may output a 16×6 transformation matrix representing each tooth in the upper arch being associated with three translation components and three rotation components of the tooth at a final position after treatment.

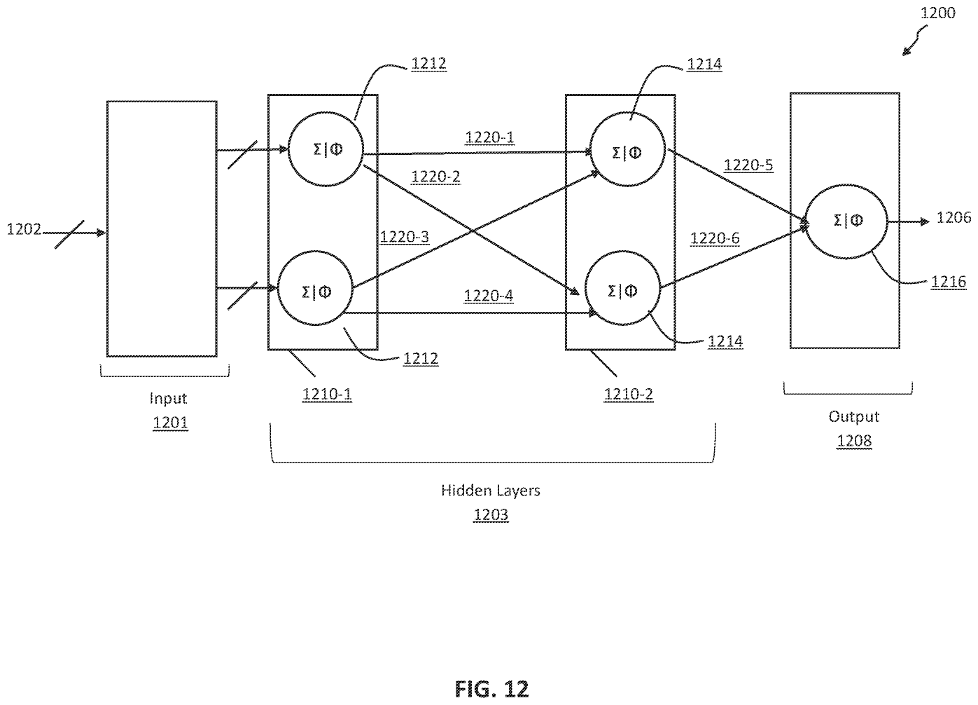

Referring next to FIG. 12, a block diagram of a simplified neural network model 1200 is shown, according to an example embodiment. The neural network may be a machine learning model that is trained to predict a final tooth position. The neural network model 1200 may include a stack of distinct layers (vertically oriented) that transform a variable number of inputs 1202 being ingested by an input layer 1204, into an output 1206 at the output layer 1208.

The neural network model 1200 may include a number of hidden layers 1210 between the input layer 1204 and output layer 1208. Each hidden layer has a respective number of nodes (1212 and 1214). In the neural network model 1200, the first hidden layer 1210-1 has nodes 1212, and the second hidden layer 1210-2 has nodes 1214. The nodes 1212 and 1214 perform a particular computation and are interconnected to the nodes of adjacent layers (e.g., nodes 1212 in the first hidden layer 1210-1 are connected to nodes 1214 in a second hidden layer 1210-2, and nodes 1214 in the second hidden layer 1210-2 are connected to nodes 1216 in the output layer 1208). Each of the nodes (1212, 1214 and 1216) sum up the values from adjacent nodes and apply an activation function, allowing the neural network model 1200 to detect nonlinear patterns in the inputs 1202. Each of the nodes (1212, 1214 and 1216) are interconnected by weights 1220-1, 1220-2, 1220-3, 1220-4, 1220-5, 1220-6 (collectively referred to as weights 1220). Weights 1220 are tuned during training to adjust the strength of the node. The adjustment of the strength of the node facilitates the neural network's ability to predict an accurate output 1206.

In some embodiments, the output 1206 may be one or more numbers (e.g., a matrix of real numbers). The one or more numbers or matrix of real numbers may be representative of tooth movements (e.g., a translation/rotation component associated with a final tooth position after treatment).

Referring back to FIG. 10, and once the final position model 1010 is trained (e.g., via historic treatment plan data), the final position model 1010 may be deployed by the final position processing engine 210 to determine the teeth movements 1012 for the final 3D representation 1016.

It is noted that, while the final position model 1010 and geometric encoder model 1006 are shown as separate models, the final position model 1010 and geometric encoder model 1006 may be sub-components or elements of a single model. For example, a machine learning model may be trained to perform the steps of both the geometric encoder model 1006 and the final position model 1010. In this regard, the final position model 1010 and geometric encoder model 1006 are shown as separate components for purposes of illustration, and the present disclosure is not limited to this particular arrangement. In some embodiments, the machine learning model may include additional or alternative models. For example, the machine learning model may also include a gingiva model configured or trained to determine or learn an evolution of a patient's gingiva during treatment. For example, the gingiva model may be trained on training data including a patient's gingiva at various points in time. The gingiva model may be trained to determine an evolution of the patient's gingiva by predicting the position of gingiva on one or more teeth. The prediction of future gingiva on a patient's teeth may be used for designing or generating aligners which fit a patient's gingiva better.

In some embodiments, the final position processing engine 210 may determine the final 3D representation 1016 by applying the teeth movements 1012 (e.g., the rigid body transformation matrix determined from the final position model 1010) to the teeth representations 1004. In some embodiments, the final position processing engine 210 may determine the final 3D representation 1016 by applying the compressed teeth representations 1008 to the geometric decoder model 1007 to decompress the compressed teeth representations and generate decompressed teeth representations 1014. In these embodiments, the final position processing engine 210 may apply the teeth movements 1012 (e.g., the rigid body transformation matrix) to the decompressed teeth representations 1014 at the initial position to generate a final 3D representation 1016 (e.g., a 3D teeth representation at a final position after a treatment plan). In some embodiments, the final position processing engine 210 may be configured to apply the rotation/translation components of one or more final tooth positions determined via teeth movements 1012 to the compressed teeth representation 1008. In these embodiments, the geometric decoder model 1007 will decompress the compressed teeth representations at the final post treatment teeth position such that the final 3D representation 1016 is the same as the decompressed teeth representation 1014.

Referring back to FIG. 10 along with FIG. 2 and FIG. 9, and following generating the final 3D representation 1016, the staging processing engine 212 may be configured to determine one or more intermediate 3D representations (or staged 3D models) between the initial 3D representation 1002 and final 3D representation 1016. The intermediate 3D representations may correspond to stages in between the initial position to the final position. For example, the intermediate 3D representation may be represented by applying half of the translation components and half of the rotation components (or some combination of components) to the initial 3D representation 1002. Following generating the intermediate 3D representations (or staged 3D models), the treatment planning computing system 102 may be configured to transmit, send, or otherwise provide the staged 3D models to the fabrication computing system 106 for manufacturing dental aligners 220 as described above.

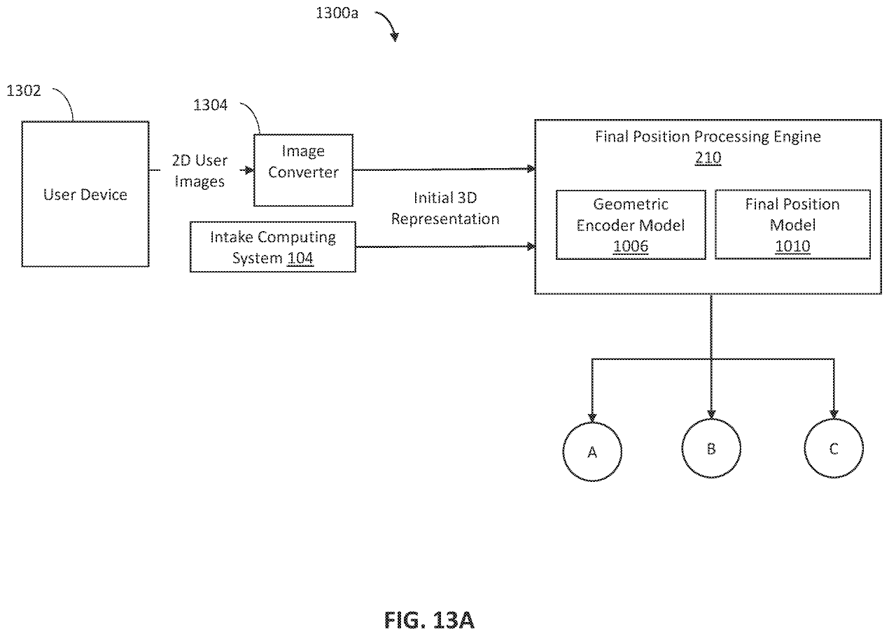

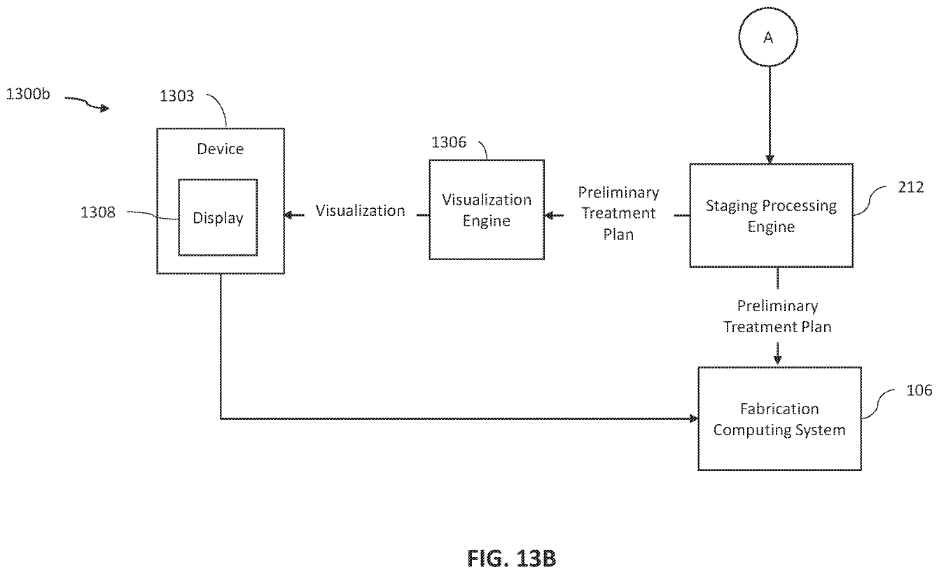

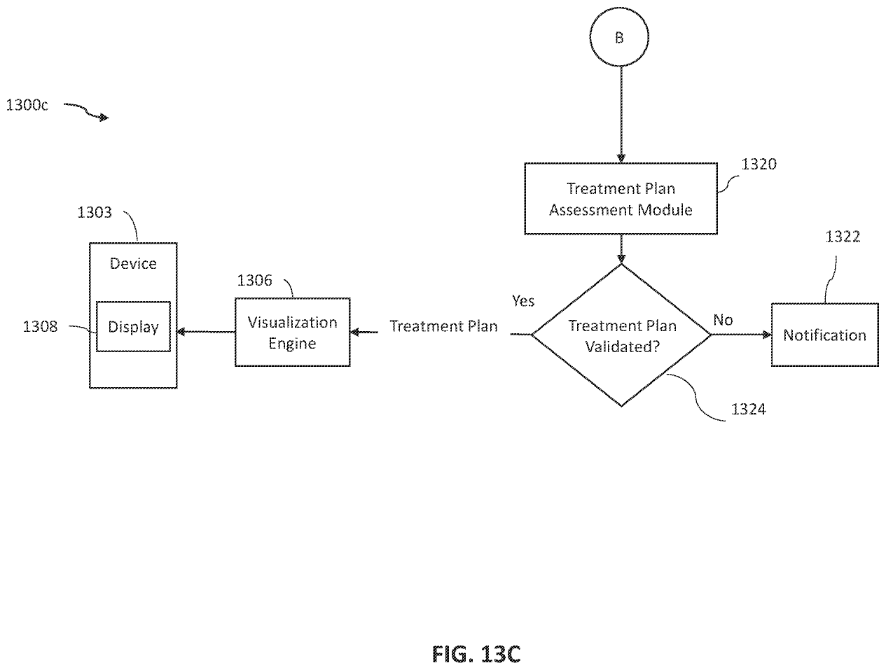

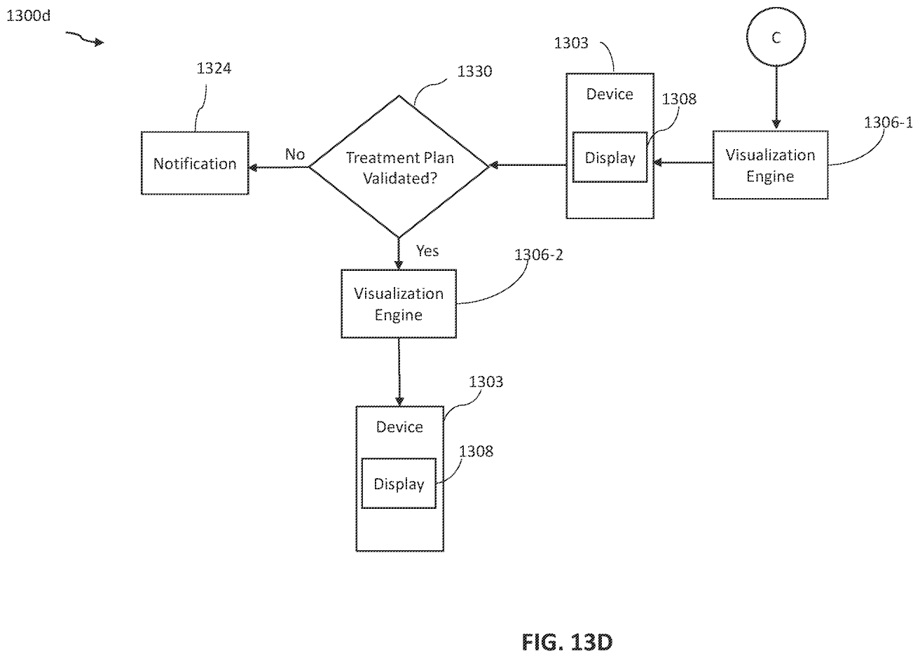

Referring now to FIGS. 13A-D, depicted are systems 1300a-1300d for generating a treatment plan, according to an illustrative embodiment. The systems 1300a-1300d may include components similar to those described above with reference to FIG. 1-FIG. 12. For example, the systems 1300a-1300d may include the intake computing system 104, the final position processing engine 210, the treatment approval terminal 109, the order/purchase terminal 111, the fabrication computing system 106 and/or the staging processing engine 212. The systems 1300a-1300d may also include a user device 1302, an image converter 1304, a visualization engine 1306, a treatment plan assessment module 1320, and/or a display 1308. The systems 1300a-1300d may be used to generate a treatment plan in real-time or near real-time.

The treatment plan may be a preliminary treatment plan similar to the treatment plan generated by the treatment planning computing system 102 described above with reference to FIG. 1-FIG. 12. For example, the preliminary treatment plan may include a preliminary final 3D representation showing a potential final position for a patient along with a series of preliminary intermediate 3D representations showing a progression from an initial position of the patient's teeth to the potential final position. Rather than using the preliminary treatment plan for treating a patient's malocclusion as is typically done with a treatment plan, the preliminary treatment plan may show a possible outcome of a patient should they undergo treatment via dental aligners 220. The treatment plan may also be a final treatment plan. The final treatment plan may be considered the treatment plan that is suitable for orthodontic treatment. That is, the final treatment plan is the treatment plan used to manufacture dental aligners to move the position of a patient's teeth from their initial position to the final after treatment position. The final treatment plan may be the same as the preliminary treatment plan if, for example, the preliminary treatment plan is validated and/or approved.

In some embodiments, a potential patient may capture 2D images of the patient's dentition using a user device 1302. In some embodiments, the potential patient may capture 2D images of a dental impression administered by the patient. The user device may be a smart phone, a camera, etc. The patient may capture a series of 2D images of the patient's dentition from various angles. The user device may upload, send, transmit, or otherwise provide the 2D images to an image converter 1304. The image converter 1304 may be configured to convert the 2D images to an initial 3D representation of the patient's dentition using photogrammetry/triangulation for instance, as discussed with reference to the scan pre-processing engine 202 in FIG. 2. The 3D representation of the patient's detention may be used for generating the treatment plan using the final position processing engine 210. The image converter 1304 may include or use one or more machine learning models, artificial intelligence, or other algorithms for converting 2D images to 3D representations.

In some embodiments, the intake computing system 104 may be configured to generate the initial 3D representation used for generating the treatment plan. For example, the intake computing system 104 may be configured to generate the initial 3D representation from an intraoral scan at an intraoral scanning site as described above with reference to FIG. 2.

The intake computing system 104 (or image converter 1304) may be configured to transmit the initial 3D representation to the final position processing engine 210 to generate a final 3D representation, as described herein. The intake computing system 104 (or image converter 1304) may be configured to transmit the initial 3D representation to the final position processing engine 210 in real-time or near-real time. While not shown, it is noted that, in some embodiments, one or more pre-processing or processing steps may be performed on the initial 3D representation (such as by the scan pre-processing engine 202, the gingival line processing engine 204, segmentation processing engine 206, and/or geometry processing engine 208 as described above with reference to FIG. 2-FIG. 7).

The final position processing engine 210 may be configured to generate a final 3D representation based on the initial 3D representation. The final position processing engine 210 may be configured to generate the final 3D representation using the geometric encoder model 1006 and final position model 1010 as described above with reference to FIG. 10-FIG. 12. In a first embodiment, the final position processing engine 210 may be configured to output the final 3D representation to the staging processing engine 212 to determine whether the final 3D representation is approved/accepted to create a treatment plan (as described in system 1300b in FIG. 13B). In a second embodiment, the final position processing engine 210 may be configured to output the final 3D representation to the treatment plan assessment module 1320 to determine whether the final 3D representation is approved/accepted to create a treatment plan (as described in system 1300c in FIG. 13C). In a third embodiment, the final position processing engine 210 may be configured to output the final 3D representation to the visualization engine 1306 to determine whether the final 3D representation is approved/accepted to create a treatment plan (as described in system 1300d in FIG. 13D).

As shown in FIG. 13B, in system 1300b, the final position processing engine 210 outputs the final 3D representation to the staging processing engine 212. The staging processing engine 212 may be configured to generate a treatment plan using the final 3D representation. The staging processing engine 212 may be configured to generate the treatment plan by generating a plurality of preliminary intermediate 3D representations representing a series of stages from the initial position shown in the initial 3D representation to a preliminary final position shown in the preliminary final 3D representation.

In some embodiments, the final position processing engine 210, the staging processing engine 212, and/or one or more other engines of the system 1300b may perform automated quality control rules or algorithms to ensure that the preliminary final 3D representation and preliminary intermediate 3D representations satisfy one or more rules. For example, the automated quality control rules or algorithms may include ensuring that collisions do not occur at any stage, or any collisions are less than a certain intrusion depth (e.g., less than 0.5 mm). The automated quality control rules or algorithms may include ensuring that certain teeth (such as centrals) are located at approximately a midline of the dentition. The system 1300 may adjust the preliminary final 3D representation and/or preliminary intermediate 3D representation based on an outcome of the automated quality control rules (e.g., to ensure that collisions satisfy the automated quality control rules, to ensure that teeth are located at approximately their intended position, etc.).

The system 1300b is shown to include a visualization engine 1306. The visualization engine 1306 may be or include any device(s), component(s), circuit(s), or other combination of hardware components designed or implemented to determine, produce, or otherwise generate a visualization corresponding to the treatment plan. The visualization engine 1306 may be a component of the treatment planning computing system 102 described above with reference to FIG. 1-FIG. 12. In some embodiments, the visualization engine 1306 may be separate from the treatment planning computing system 102. The visualization generated by the visualization engine 1306 may show a progression from the initial 3D representation (e.g., the teeth at an initial position in a patient's mouth), through the preliminary intermediate 3D representations, and to the final 3D representation (e.g., the teeth at the final position in the patient's mouth). Additionally or alternatively, the visualization may also show the stages of the treatment plan. For example, the visualization engine 1306 may show an initial stage of the treatment plan corresponding to a first 3D model and/or dental aligners corresponding to the initial stage of the treatment plan, one or more intermediate stages of the treatment plan corresponding to one or more intermediate 3D models and/or dental aligners for the intermediate stages of the treatment plan, and a final stage of the treatment plan corresponding to a final 3D model and/or dental aligners for the final stage of the treatment plan. In some embodiments, the visualization engine 1306 may show the final position of teeth after the treatment plan (via a 3D representation).

The visualization engine 1306 may be configured to generate the visualization for rendering on a display 1308 of a device 1303. The visualization engine 1306 may be configured to receive the treatment plan from the staging processing engine 212, and generate the visualization from the treatment plan. The visualization engine 1306 may be configured to generate the visualization as a video, a series of 2D images, or other graphical/visual representation of the treatment plan. The visualization engine 1306 may be configured to transmit, send or otherwise provide the visualization for displaying on a display 1308. In some embodiments, the display 1308 may be a display of the user device 1302. In this regard, the visualization engine 1306 may transmit the visualization back to the user device 1302 (e.g., which uploaded the 2D user images to the image converter 1304) for displaying on the display 1308. The visualization engine 1306 may transmit the visualization to the user device 1302 using an email address or phone number provided by a user when uploading the 2D user images, to a user portal accessible by a user of the user device 1302 via log-in credentials, etc. In some embodiments, the display 1308 may be a display of a computing device at an intraoral scanning site (e.g., an orthodontist office). In this regard, the visualization engine 1306 may be configured to transmit the visualization to a computing device at the intraoral scanning site for displaying on the display 1308. The display 1308 may be located in a room or space in which a user has received an intraoral scan.

The visualization engine 1306 may be configured to transmit the visualization for displaying on the display 1308 in real-time or near real-time. The visualization may therefore show a potential or preliminary visualization of a possible or estimated treatment outcome if the patient (or user) were to be treated via dental aligners 220. As such, the patient may view the visualization on device 1303 (e.g., user device 1302) and determine whether to obtain treatment via dental aligners 220 and/or approve the treatment plan. Similarly, an administrator/technician may view the visualization on device 1303 (e.g., treatment planning terminal 108) and determine whether the dental aligners 220 and/or treatment plan are acceptable (or approved) for treatment.

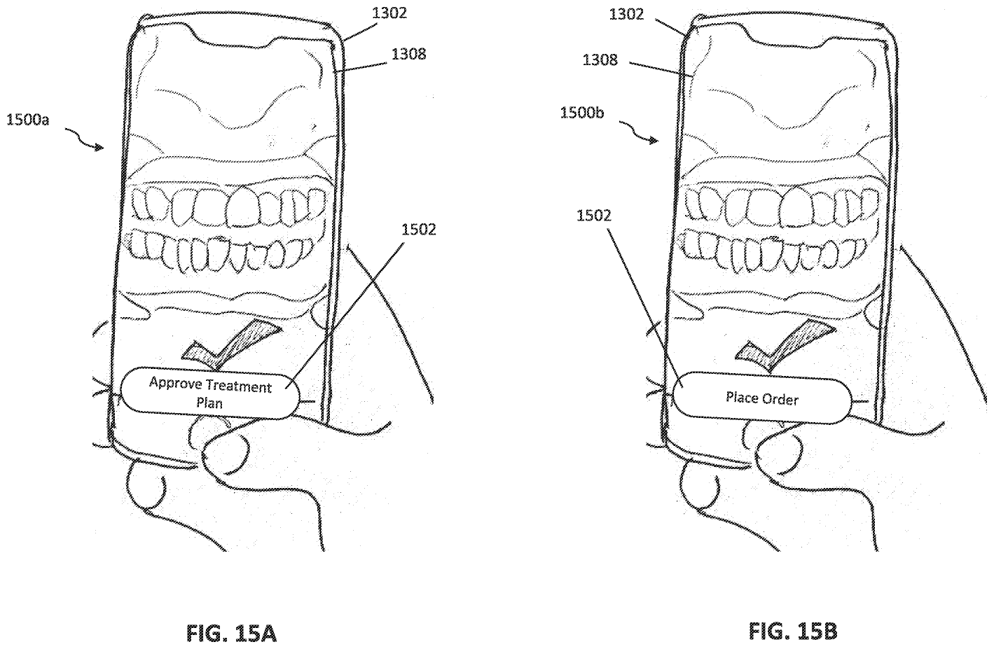

The patient and/or administrator may view the visualization shortly after capturing the 2D user images and/or receiving the intraoral scan. In some embodiments, the treatment plan is a preliminary treatment plan and may not be used for generating the final (e.g., actual) treatment plan for the user. In this regard, the visualization may serve to provide relatively-instantaneous information regarding a potential outcome of treatment, which may assist a user/patient in deciding whether to undergo treatment via dental aligners. In some embodiments, the treatment plan may be used as the final (e.g., actual) treatment plan for the user. For example, the treatment plan may become the final treatment plan after the preliminary treatment plan has been approved by the user (e.g., via the user device 1302) and/or an administrator/technician at the intraoral scanning site via the treatment planning terminal 108). For instance, responsive to the user (and, in some cases, the administrator) approving the treatment plan, the treatment plan may become the final treatment plan and the fabrication computing system 106 may initiate the manufacture/printing/fabrication of the aligners 220. The user may approve/acknowledge the treatment plan by indicating that the user would like to purchase the aligners 220 (e.g., interacting with an “Order Now” button or “Pay Now” button, interacting with a slider, interacting with an object, communicating audibly, communicating with gestures). Referring to FIGS. 15A-15B, depicted are examples of a user approving/acknowledging the treatment plan. As shown, the user device 1302 is displaying a final 3D representation to the user via display 1308. The display 1308 includes interactive button 1502 which indicates that the user approves the final 3D representation (or intends to place an order). In some implementations, the interactive button 1502 may indicate that the user does not approve of the final 3D representation (e.g., the interactive button may communicate “Don't Like”). In yet other implementations, the interactive button 1502 may be a button (or calendar, automated number dialer, and the like) allowing the user to communicate with an office to book an appointment with a treating dentist, technician, orthodontist, administrator, and the like. In other implementations, the display 1308 may communicate multiple interactive buttons that evaluate whether the user accepts/approves of the final 3D representation. In some cases, the user may not be able to order/purchase the dental aligners 220 until an administrator has approved the treatment plan. The user's interaction with interactive button 1502 on the user device 1302 creates an order and/or purchase that is transmitted to an order/purchase terminal (e.g., order/purchase terminal 111 of FIG. 1) for storage/subsequent processing of the order/purchase (e.g., an initiation of a payment/order completion process, as described with reference to FIG. 1).

Referring back to FIG. 13B, in an alternate example, the treatment plan may become the final treatment plan responsive to the final position processing engine 210 satisfying one or more criteria (e.g., a threshold number of users (e.g., technicians, administrators, treating dentists) have approved the treatment plan without modifications, a threshold number of positive reviews regarding the treatment plan from patients). The final position processing engine 210 satisfying the one or more criteria may indicate that the final position processing engine 210 is sufficiently accurate in predicting the final teeth positions.

The system 1300b is also shown to include a fabrication computing system 106. As described herein, the fabrication computing system 106 can include a fabrication computing device and fabrication equipment 218 configured to produce, manufacture, or otherwise fabricate dental aligners 220 corresponding to each stage of the treatment plan where each stage may be representative of a particular 3D model determined by the final 3D representation.

In some embodiments, the fabrication computing system 106 may receive the treatment plan from the staging processing engine 212 and fabricate dental aligners 220 without any user intervention from the user device 1302 and/or from the treatment planning terminal 108 (e.g., a semi-automated or fully automated treatment planning process). Therefore, the treatment plan may be considered the final treatment plan. In some embodiments, the fabrication computing system 106 may be configured to fabricate dental aligners 220 after receiving approval (or acknowledgement) from the user (e.g., via the user device 1302) and/or an administrator/technician at the intraoral scanning site via the treatment planning terminal 108).

As shown in FIG. 13C, the system 1300c describes a fully automated system for verifying the safety and clinical efficacy of an orthodontic treatment plan by performing a clinical assessment on the treatment plan using insights typically obtained and verified during a diagnosis by a professional in the field. The approval/validation of the final tooth position post treatment is automated, as described below. The final position processing engine 210 outputs the final 3D representation to the treatment plan assessment module 1320. The treatment plan assessment module 1320 may include the same or similar components, circuits, hardware, and/or logic as the staging processing engine 212 to convert the 3D representation into a treatment plan. Accordingly, the treatment plan assessment module 1320 may generate the treatment plan (e.g., an initial stage of the treatment plan corresponding to a first 3D model and/or dental aligners for the initial stage of the treatment plan, one or more intermediate stages of the treatment plan corresponding to one or more intermediate 3D models and/or dental aligners for the intermediate stages of the treatment plan, and a final stage of the treatment plan corresponding to a final 3D model and/or dental aligners for the final stage of the treatment plan) and subsequently assess the treatment plan. In some implementations, the final position processing engine 210 outputs the final 3D representation to the staging processing engine 212 to generate the treatment plan and subsequently transmits the generated treatment plan to the treatment plan assessment module 1320. Accordingly, the treatment plan assessment module 1320 may receive one or more stages of the treatment plan. In some implementations, the treatment plan assessment module 1320 may receive one or more stages of the treatment plan and the 3D representation of the teeth.

The treatment plan assessment module 1320 may be any device(s), component(s), circuit(s), or other combination of hardware components designed or implemented to assess the treatment plan. For example, the treatment plan assessment module 1320 may employ rule based logic (e.g., if-then rules) and/or machine learning to assess the treatment plan. For example, a machine learning model may be trained to classify whether a treatment plan is a valid treatment plan given historic treatment plans (e.g., historic treatment plans and corresponding classifications of the validity of the treatment plan). The machine learning model may be a neural network, a random forest, support vector machines, and the like. Additionally or alternatively, the treatment plan assessment module 1320 may obtain one or more metrics from the treatment plan (using one or more engines, as described herein) and compare the metric(s) to criteria. The treatment plan assessment module 1320 assesses the validity of the treatment plan using rule based logic based on the comparison.

In a non-limiting example, the treatment plan assessment module 1320 may identify the positions of the teeth in a received final stage of treatment planning and determine a grading of the treatment plan. If the grading satisfies one or more criteria, the treatment plan assessment module 1320 may determine that the treatment plan is valid.

In another non-limiting example, the treatment plan assessment module 1320 may obtain one or more metrics associated with the treatment plan. The treatment plan assessment module 1320 compares the metrics against one or more criteria. An example of a criterion is whether a smile is aesthetically pleasing. One example metric obtained from the treatment plan and used in the evaluation of whether a smile is aesthetically pleasing may the position of the incisal edges. A smile may be determined to be aesthetically pleasing if the incisal edges are aligned (e.g., the incisal edges may be used by the treatment plan assessment module 1320 to determine the smile line). If the incisal edges are within a predetermined range, the treatment plan assessment module 1320 may determine that the incisal edges are aligned and subsequently that the smile is aesthetically pleasing.

Another metric that may be obtained from the treatment plan by the treatment plan assessment module 1320 may include an evaluation of the occlusion. The treatment plan assessment module 1320 may obtain the occlusion of the individual teeth for the final teeth position (using the treatment plan and/or final 3D representation). If the identified occlusion satisfies one or more criteria, the treatment plan assessment module 1320 may determine that the treatment plan is valid.

The one or more metrics obtained from the treatment plan assessment module 1320 will be used to evaluate whether the treatment plan is a valid treatment plan at 1324 (e.g., whether the treatment plan is a clinically sound and biologically sensible treatment plan). As described herein, the treatment plan assessment module 1320 will determine whether the treatment plan is valid (e.g., at 1324) by comparing the metric(s) to one or more criteria. In the first non-limiting example, the treatment plan is validated if the smile resulting from the treatment plan (e.g., the smile produced by the teeth at the final positions post treatment) is aesthetically pleasing (e.g., the incisal edge alignment satisfies one or more criteria). In the second non-limiting example, the treatment plan is validated if the occlusion is determined to be clinically correct (e.g., satisfying one or more criteria). Accordingly, the treatment plan assessment module 1320 will be used to determine (via the treatment plan validation step 1324) whether the resulting final teeth position, indicated by the 3D representation and/or the final stage, has addressed any previous potential malocclusion of the teeth.

If the treatment plan is validated at 1324, the treatment plan (and/or the 3D representation) may be transmitted to the visualization engine 1306. As described herein, the visualization engine may show the treatment plan (e.g., the final stage), stages of the treatment plan, and/or a 3D representation of the treatment plan (or stages of the treatment plan) to a device 1303 to be displayed via display 1308. The device may be a user device 1302 and/or a treatment approval terminal 109. A user (e.g., treating dentist, administrator, technician, patient) may use the user device 1302 and/or treatment approval terminal 109 to view the displayed and validated treatment plan (e.g., the final stage), stages of the treatment plan, and/or a 3D representation of the treatment plan (or stages of the treatment plan). For example, a patient may use user device 1302 to order the treatment plan, make a payment, approve the treatment plan, initiate the production of one or more dental aligners based on the treatment plan, or some combination. In these embodiments, the patient's order/purchase is transmitted to an order/purchase terminal (e.g., order/purchase terminal 111 of FIG. 1) for storage/subsequent processing of the order/purchase.

If the treatment plan is not validated at 1324, the treatment plan assessment module 1320 may generate a notification 1322 to be transmitted to one or more users (e.g., a patient using user device 1302 and/or a treating dentist using device 1303). The notification may communicate (e.g., using the display of the device 1303, using a microphone of device 1303/1302) that the generated treatment plan was not validated. In one embodiment, the notification may include a reason and/or feedback on why the treatment plan was not validated, and may also include recommendations or requirements to update the treatment plan in such a way that it can be validated and accepted. As an example, such feedback may include a request to move one or more teeth in a way that is different to the preliminary treatment plan. The treatment plan assessment module 1320 may create the recommendation/feedback using a machine learning model. For example, a machine learning model may be trained to recommend an improvement to a treatment planning model given historic treatment plans (e.g., historic treatment plans and corresponding historic improvements recommended for the treatment plan by a user). In some implementations, the treatment plan assessment module 1320 may prompt a user (e.g., the treating dentist and/or a patient) for additional 2D images of the patient's dentition and/or additional scans of the patient's dentition. In other implementations, the notification 1322 may prompt a user to take additional action(s). For example, a treating dentist may be prompted by the treatment plan assessment module 1320 to edit the treatment plan. Additionally or alternatively, a patient may be prompted to call the treating dentist for additional orthodontic solutions/options. In some implementations, each time a treatment plan is not validated, the treatment plan assessment module 1320 may increment a counter. When the counter reaches and/or exceeds a threshold value, the treatment plan assessment module 1320 may trigger the final position processing engine 210 to retrain the geometric encoder model 1006 and/or the final position model 1010.

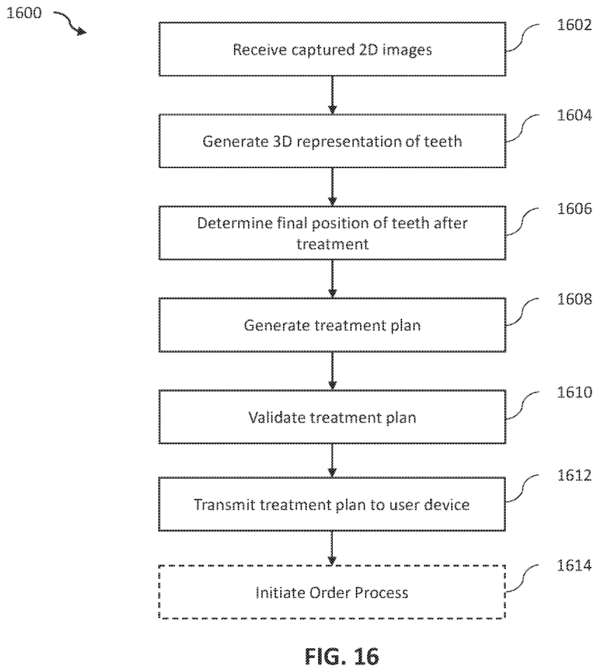

Referring now to FIG. 16, depicted is a flowchart showing a method 1600 of the system 1300c in FIG. 13C. Method 1600 describes automatically verifying the safety and clinical efficacy of the orthodontic treatment plan by performing a clinical assessment on the treatment plan. As a brief overview, at step 1602, one or more processors receive captured 2D images. At step 1604, the processor(s) generate 3D representations of teeth. At step 1606, the processor(s) generate a final position of teeth after treatment. At step 1608, the processor(s) generate a treatment plan. At step 1610, the processor(s) validate the treatment plan. At step 1612, the processor(s) transmit the treatment plan to a user device. At step 1614, the processor(s) initiate an order process. The method 1600 including each of the steps 1602-1614 may be performed by one or more of the devices or components described above with reference to FIG. 1-FIG. 13. Additionally, while shown as being performed in a particular order, it is noted that the steps of the method 1600 may be performed in any order.

At step 1602, one or more processor receive captured 2D images. A patient may capture 2D images of the patient's dentition using a user device 1302. In some embodiments, the patient may capture 2D images of a dental impression administered by the patient. The user device 1302 may be a smart phone, a camera, etc. The patient may capture a series of 2D images of the patient's dentition from various angles. The patient device may upload, send, transmit, or otherwise provide the captured 2D images to the processor(s) such that the processor(s) receive the captured 2D images.

At step 1604, the processor(s) generate 3D representations of teeth. The processor(s) generate 3D representations of teeth using the scan pre-processing engine 202 as described above with respect to FIG. 2. The scan pre-processing engine 202 generates 3D representations of the 2D images using photogrammetry and/or triangulation as described above. Additionally or alternatively, the processor(s) generate 3D representations of the teeth using an image converter.

At step 1606, the processor(s) determine the final position of the teeth after treatment. The processor(s) may determine the final position of the teeth after treatment using the final position processing engine 210 as described above with respect to FIG. 1-FIG. 12. The final position processing engine 210 may feed the 3D representations of teeth at an initial position to geometric encoder model 1006. The geometric encoder model 1006 is an autoencoder and trained using unsupervised based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation). The encoded latent space representation of the one or more teeth may be fed to the final position model 1010 to determine the movement of teeth of a detention from initial positions to final positions and/or a 3D representation of teeth at a final position post treatment. The final position model 1010 may be trained on a training set including a plurality of compressed 3D training representations of dentitions comprising a plurality of teeth, and corresponding tooth movements to respective final positions.

At step 1608, the processor(s) generate a treatment plan. The staging processing engine 212 is configured to generate stages of treatment (e.g., a treatment plan) from the initial position to the final position of the patient's teeth. For example, the staging processing engine 212 generates the stages as 3D digital models of the patient's teeth as the teeth progress from their initial position to their final position, where each 3D digital model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

At step 16010, the processor(s) validate the treatment plan. The treatment plan assessment module 1320 is configured to validate the treatment plan. The treatment plan assessment module 1320 assesses/evaluates the validity of the treatment plan using rule based logic and/or machine learning to classify the validity of the treatment plan.

At step 1612, the processor(s) transmit the validated treatment plan to a user device. A visualization engine 1306 may show the treatment plan (e.g., the final stage), stages of the treatment plan, and/or a 3D representation of the treatment plan (or stages of the treatment plan) to a device 1303 to be displayed via display 1308.

At optional step 1614, the processor(s) may receive an input initiating an order process. The input initiating the order process includes an input from user device 1302 by a patient (or potential patient) to order the treatment plan, make a payment, approve the treatment plan, initiate the production of one or more dental aligners based on the treatment plan, or some combination. The patient's order/purchase is transmitted to an order/purchase terminal (e.g., order/purchase terminal 111 of FIG. 1) for storage/subsequent processing of the order/purchase. The order/purchase terminal 111 communicates prompts to the user device 1302 asking the patient for patient information (e.g., name, physical address, email address, phone number, credit card information) and product information (e.g., quantity, product name) to guide the patient through a payment process.

Referring to FIG. 13D, the system 1300d describes a semi-automatic system for allowing a reviewer to approve a treatment plan. The approval/validation of a final tooth position post treatment is determined using a manual review/approval by a treating dentist as described below. The final position processing engine 210 outputs the final 3D representation to the visualization engine 1306-1.

The visualization engine 1306-1 may include the same or similar components, circuits, hardware, and/or logic as the staging processing engine 212 to convert the 3D representation into a treatment plan. Accordingly, the visualization engine 1306-1 may generate the treatment plan and transmit the treatment plan to device 1303 to be displayed on display 1308. In some implementations, the final position processing engine 210 outputs the final 3D representation to the staging processing engine 212 to generate the treatment plan and subsequently transmits the generated treatment plan to the visualization engine 1306-1. Accordingly, the visualization engine 1306-1 may receive one or more stages of the treatment plan. In some implementations, the visualization engine 1306-1 may receive one or more stages of the treatment plan and the 3D representation of the teeth.

As described herein, the visualization engine 1306-1 may be configured to generate the visualization of the one or more stages of the treatment plan and/or the 3D representation of the teeth as a video, a series of 2D images, or other graphical/visual representation and display such visualization to the display 1308 on device 1303. A technical user (such as a technician, treating dentist, orthodontist, or state licensed professional that has the rights to provide orthodontic treatment to a patient) may review the visualization displayed on display 1308. The technical user may determine whether the treatment plan and/or the 3D representation of the teeth is valid at decision 1330. A treatment plan assessment module (not shown) configured to store the technical user input (e.g., transmitted from a treatment approval terminal 109) may be configured to receive other inputs from the technical user such as improve, request changes, and/or reject the treatment plan and/or the 3D representation of the teeth.

The technical user may input an approval to the treatment plan assessment module if the treatment plan and/or the 3D representation of the teeth are approved by the technical user at 1330. Subsequently, the treatment plan assessment module may transmit and/or apply the treatment plan and/or the 3D representation of the teeth to a visualization engine 1306-2. The visualization engine 1306-2 may convert, transform, reformat or otherwise modify the treatment plan and/or the 3D representation of the teeth such that the treatment plan and/or the 3D representation of the teeth are in a state/format suitable for viewing by a patient/potential patient (e.g., a person considering receiving dental treatment) on device 1303 via display 1308. In some embodiments, after the treatment plan is validated at 1330 by a technical user, the treatment plan may be transmitted to device 1303 (e.g., there may be no visualization engine 1306-2). In some embodiments, the visualization engine 1306-2 may supplement the treatment plan and/or 3D teeth representations visualized by visualization engine 1306-1. For example, the visualization engine 1306-1 may visualize the 3D representation of the teeth to the user device 1303 for review by a technical user. The visualization engine 1306-2 may subsequently visualize the treatment plan, including the initial stage including the 3D digital model of the patient's teeth at their initial position, one or more intermediate stages including 3D digital model(s) of the patient's teeth at one or more intermediate positions, and the final stage including a 3D digital model of the patient's teeth at the final position. Accordingly, the visualization engine 1306-2 may visualize the treatment plan and the 3D teeth representation to be displayed to user device 1302 (e.g., a patient cell phone) via display 1308.

The patient is able to view the 3D teeth representation and/or the treatment plan, in addition to other information relating to the treatment plan (e.g., tooth movements, tooth rotations and translations, clinical indicators, the duration of the treatment plan, the orthodontic appliance that is prescribed to achieve the final tooth position (e.g., aligners), the recommended wear time of the appliance to affect the final tooth position). The information related to the treatment plan may be determined based on historic treatment plans. For example, clinical indicators, the duration of the treatment plan, the orthodontic appliance that is prescribed to achieve the final tooth position (e.g., aligners), and the recommended wear time of the appliance to affect the final tooth position may be determined from a historic treatment plan with a similar initial position and similar final position. Similarly, the information relating to the treatment plan may take into account biomechanical and biological parameters (collectively referred to herein as “treatment parameters”) relating to tooth movement, such as the amount and volume of tissue or bone remodeling, the rate of remodeling or the relating rate of tooth movement.

The patient may also order the treatment plan, make a payment, approve the treatment plan, initiate the production of one or more dental aligners based on the treatment plan, book an appointment with a treating dentist, order a product (e.g., an impression kit, aligner) or some combination. In these embodiments, the patient's order/purchase is transmitted to an order/purchase terminal (e.g., order/purchase terminal 111 of FIG. 1) for storage/subsequent processing of the order/purchase.

If the treatment plan is not approved by the technical user at 1330, the technical user will not input an approval to the treatment plan assessment module (not shown) using the treatment approval terminal 109. In response to not receiving an approval (e.g., receiving a denial, receiving no response), the treatment plan assessment module 1320 will generate a notification 1322. The notification 1322 may communicate (e.g., using the display of the device 1308, using a microphone of device 1303) a reminder to the technical user to evaluate the treatment plan and/or a recommendation of an improvement to the treatment plan. The treatment plan assessment module 1320 may create a recommendation of an improvement to the treatment plan using a machine learning model. For example, a machine learning model may be trained to recommend an improvement to a treatment planning model given historic treatment plans (e.g., historic treatment plans and corresponding historic improvements recommended for the treatment plan by a user). In another embodiment, if the treatment plan is not approved by the technical user at 1330, the treatment plan can be routed to be revised or a new treatment plan generated by a user such as a setup technician, an orthodontist or any other person trained to generate a treatment plan. For instance, the treatment plan assessment module 1320 may route the treatment plan to a treatment planning terminal 108.

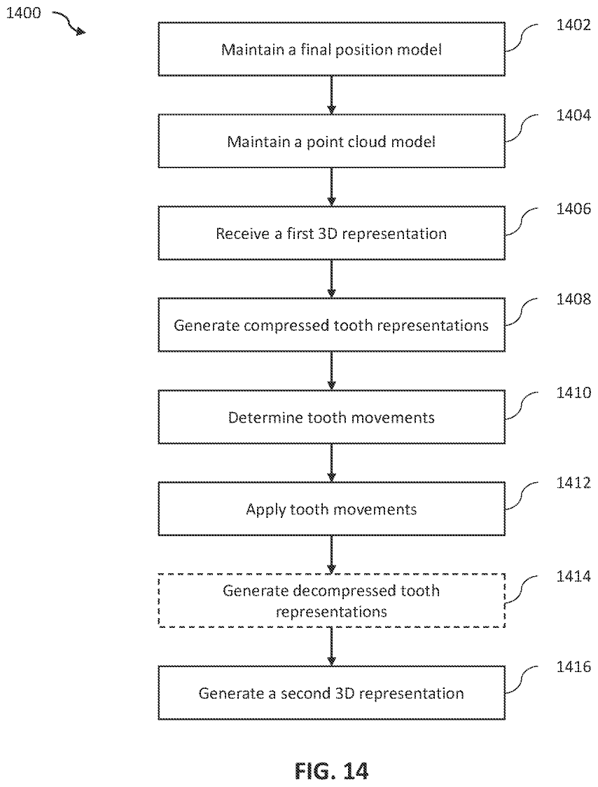

Referring now to FIG. 14, depicted is a flowchart showing a method 1400 of automatically determining a final position of a patient's dentition, according to an illustrative embodiment. As a brief overview, at step 1402, one or more processors maintain a final position model. At step 1404, the processor(s) maintain a geometric encoder model. At step 1406, the processor(s) receive a first three-dimensional (3D) representation. At step 1408, the processor(s) generate compressed tooth representations. At step 1410, the processor(s) determine tooth movements. At step 1412, the processor(s) apply tooth movements. At step 1414, the processor(s) generate decompressed tooth representations. At step 1416, the processor(s) generate a second 3D representation. The method 1400 including each of the steps 1402-1416 may be performed by one or more of the devices or components described above with reference to FIG. 1-FIG. 12. Additionally, while shown as being performed in a particular order, it is noted that the steps of the method 1400 may be performed in any order.

At step 1402, one or more processors maintain a final position model (e.g., final position model 1010). In some embodiments, the processor(s) may maintain a final position model configured to determine movement of teeth of a dentition from initial positions to final positions and/or configured to determine a 3D representation of teeth at a final position post treatment. The processor(s) may be a component or element of the treatment planning computing system 102 described above, such as the final position processing engine 210. The final position model 1010 may be trained on a training set including a plurality of compressed three-dimensional (3D) training representations of dentitions comprising a plurality of teeth, and corresponding tooth movements to respective final positions. In some embodiments, the training set may be limited by previous treatment plans objectively deemed successful. For example, the training set may be limited to treatment plans which did not result in a mid-course correction (e.g., a subsequent treatment plan generated for a patient where the patient's teeth deviated from their intended position at some stage of the treatment plan). The final position model 1010 may be trained as described above with respect to FIG. 11-FIG. 12.

At step 1404, the processor(s) maintain a geometric encoder model 1006. In some instances, the processor(s) may also maintain a geometric decoder model 1007. In some embodiments, the geometric encoder model 1006 is an autoencoder and trained using unsupervised based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation). For example, the target value (e.g., the decompressed tooth representations following compression, encoding, and subsequent decompression) is set to equal the input (e.g., the tooth representation). Accordingly, the loss function that trains the geometric encoder model 1006 is the 3D error of the reconstructed full 3D geometry of each tooth (or groups of teeth).

At step 1406, the processor(s) receive a first three-dimensional (3D) representation. In some embodiments, the processor(s) may receive a first 3D representation of a dentition including a plurality of teeth of a patient in an initial position. The 3D representation may include a plurality of tooth representations including a plurality of points representing surfaces of a respective tooth of the dentition (e.g., a point cloud representation). The first 3D representation may also include a plurality of tooth representations in a mesh representation or voxel (e.g., volumetric pixel) representation, spline representation, or other parametric model representation. In some embodiments, the first 3D representation may be obtained based on a dental impression administered by the patient or an intraoral scan. The first 3D representation may be or include a decompressed, uncompressed, or full 3D representation of the patient's dentition. The first 3D representation (including tooth representations) may include point clouds having points representative of various surfaces of the patient's dentition.

At step 1408, the processor(s) generate compressed tooth representations using the maintained geometric encoder model in step 1404. In some embodiments, the processor(s) may generate a compressed tooth representation for each tooth representation of the first 3D representation. In some embodiments, the processor(s) may generate a compressed tooth representation from groups of teeth of the first 3D representation.

At step 1410, the processor(s) determine tooth movements. In some embodiments, the processor(s) may determine tooth movements (e.g., translation components and rotation components) of the plurality of teeth of the dentition from the initial position to a final position. The processor(s) may determine the tooth movements responsive to applying each compressed tooth representation generated at step 1408 to the final position model 1010.

At step 1412, the processor(s) apply tooth movements. In some embodiments, the processor(s) apply the tooth movements to the compressed tooth representations of the respective teeth of the plurality of teeth, to move the compressed tooth representations into the final position post treatment. In some embodiments, the processor(s) may apply a rigid body transformation including three rotation components and three translation components to the compressed tooth representations using the three translation components and the three rotation components determined at step 1410. In some embodiments, the processor(s) apply the tooth movements to decompressed tooth representations (e.g., following step 1414 described below). In other words, steps 1412 and 1414 may be performed in any particular order. The processor(s) may apply the rigid body transformation to the compressed tooth representations to move the teeth according to the determined tooth movements determined at step 1410. In some embodiments, the processor(s) apply the tooth movements to the first 3D representation (e.g., the first 3D representation received in step 1406). The processor(s) may apply the rigid body transformation to move the teeth into the final post treatment position determined by the final position model 1010.

At step 1414, the processor(s) may generate decompressed tooth representations. For example, the processor(s) may generate decompressed tooth representations if the tooth movements were applied to compressed tooth representations. In some embodiments, the processor(s) may generate decompressed tooth representations using the geometric encoder model 1006. The processor(s) may apply the compressed tooth representations (e.g., determined or generated at step 1408) to a geometric decoder model 1007 to generate the decompressed tooth representations. The geometric decoder model 1007 may be configured to receive the compressed tooth representation and generate decompressed (or full) tooth representations. In some embodiments, the geometric decoder model 1007 may generate the decompressed tooth representations following the determined tooth movements being applied to the compressed tooth representations. In some embodiments, the geometric decoder model 1007 may generate the decompressed tooth representations prior to the determined tooth movements being applied to the compressed tooth representations. In this example, where the geometric decoder model 1007 generates the decompressed tooth representations prior to the determined tooth movements being applied, step 1414 may occur prior to step 1412 described above.

At step 1416, the processor(s) generate a second 3D representation. In some embodiments, the processor(s) may generate a second 3D representation of the dentition comprising the plurality of teeth of the patient in the final position. The processor(s) may generate the second 3D representation using the decompressed tooth representations generated at step 1414. The processor(s) may generate the second 3D representation using the decompressed tooth representations generated at step 1414 with the tooth movements being applied at step 1412. The processor(s) may generate the second 3D representation for rendering on a treatment planning terminal 108. The processor(s) may generate the second 3D representation for generating a treatment plan including stages of the treatment plan.

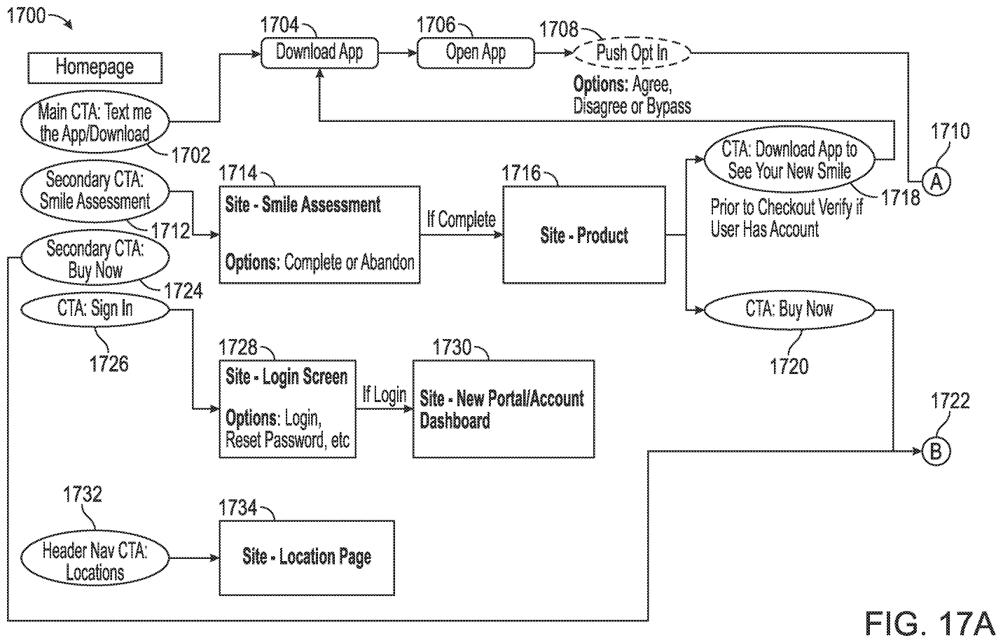

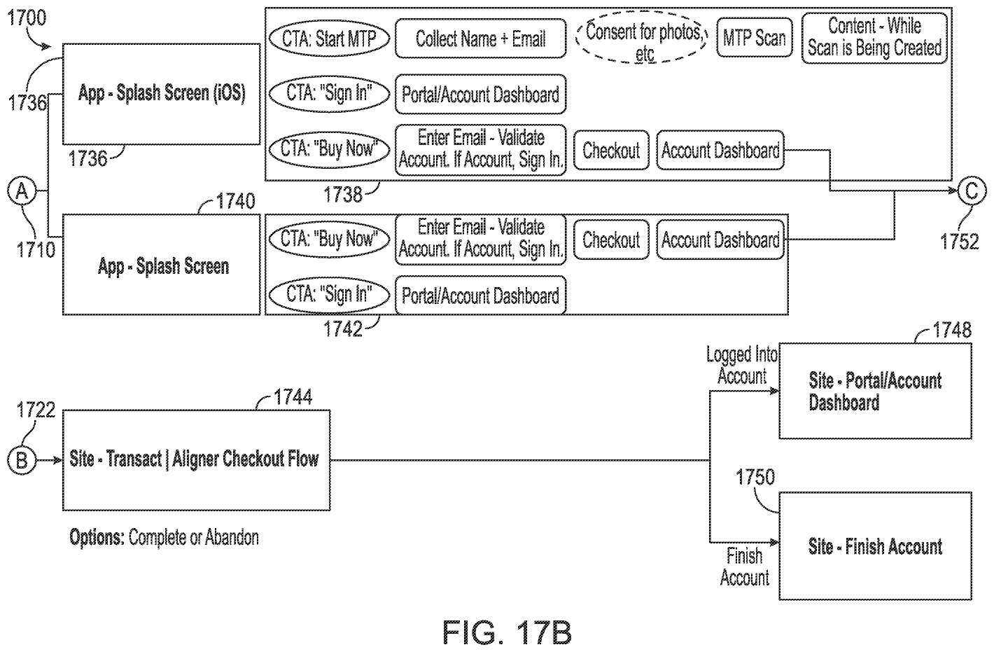

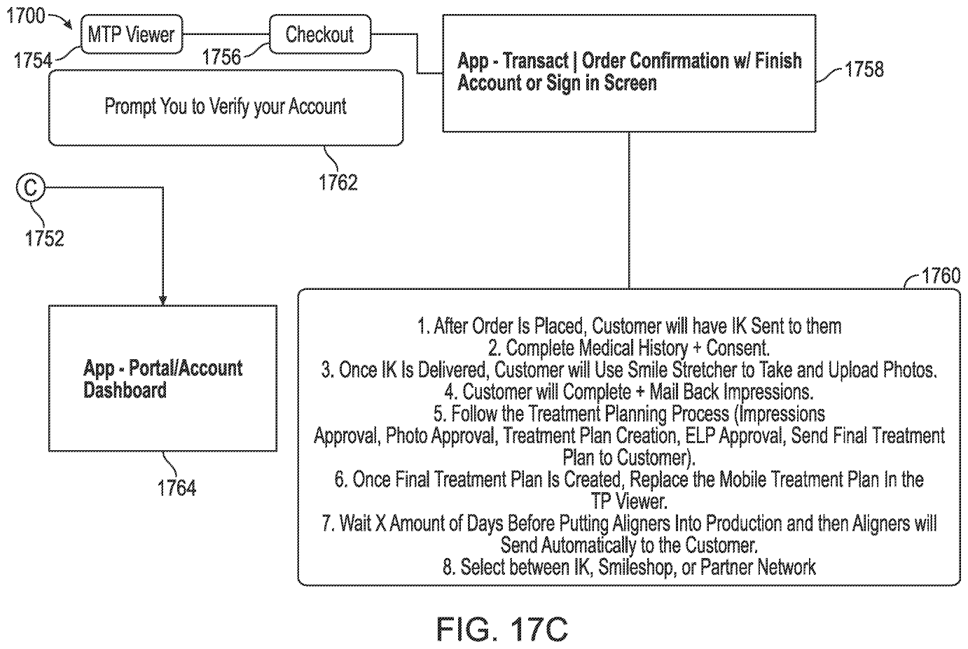

FIGS. 17A-17C are block diagrams depicting an example of a treatment plan interface architecture 1700, according to illustrative embodiments. User device 1302 or 1902 can execute and present interfaces (e.g., treatment plan interface 1901) based on the treatment plan interface architecture 1700. For example, a treatment plan interface 1901 can be executed based on receiving information from the various systems described herein. The treatment plan interface architecture 1700 is shown to include a first flow (sometimes referred to herein as “a call to action” (“CTA”)) starting at block 1702 where a user operating user device 1902 can receive a text for an application download. For example, the application may be configured to present a treatment plan interface 1901 as described in detail with reference to FIGS. 19A-19I, 20A-20C, 21A-21J, and 23A-23G. At block 1704-1708, the user can download the application, open the application, and opt in (e.g., agree, disagree, or bypass) to any privacy settings or data collection settings. Next, the user can proceed to block 1736 or 1740 based on the type of user device 1902 (e.g., mobile device operating system (OS), tablet OS, etc.). For example, at block 1736 a treatment plan interface 1901 can be presented allowing a user to select from a plurality of actionable objects (described in detail with reference to FIGS. 19A-19I, 20A-20C, 21A-21J, and 23A-23G) such as, but not limited to, a start mobile treatment plan (MTP), a sign in, or a buy now option. In block 1738, a selection of start MTP can include collecting name and email and enable the user to consent to have photos and video captured and health information stored. As used herein, the MTP can be a preliminary treatment plan depicting a final position of the patient's dentition from an initial position (as described in detail with reference to FIGS. 7-9). In some implementations, the MTP can be a final treatment plan.

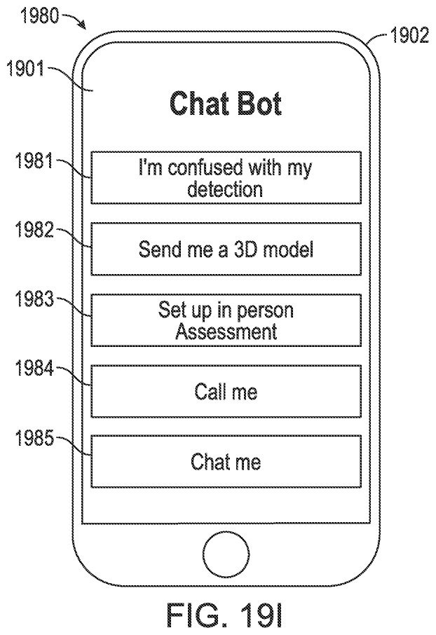

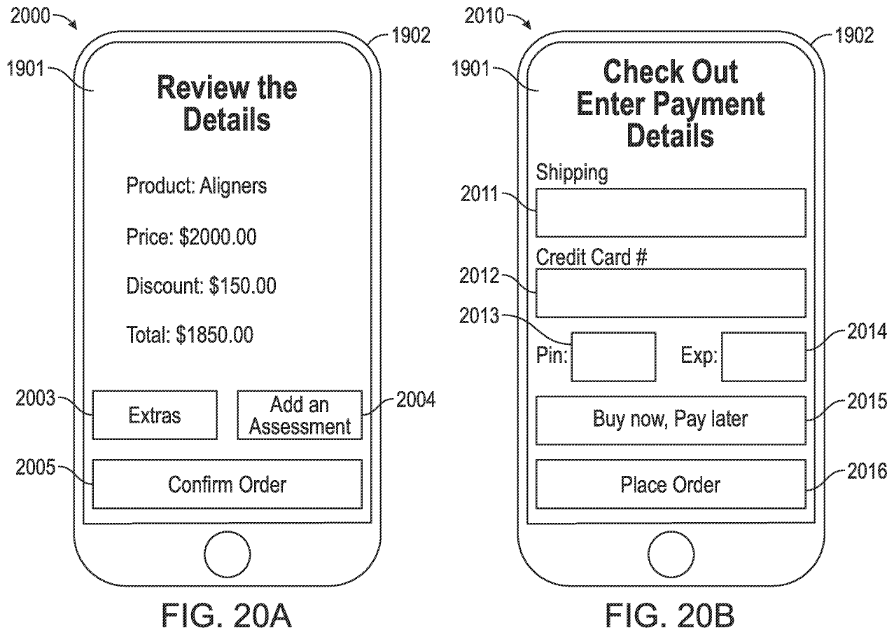

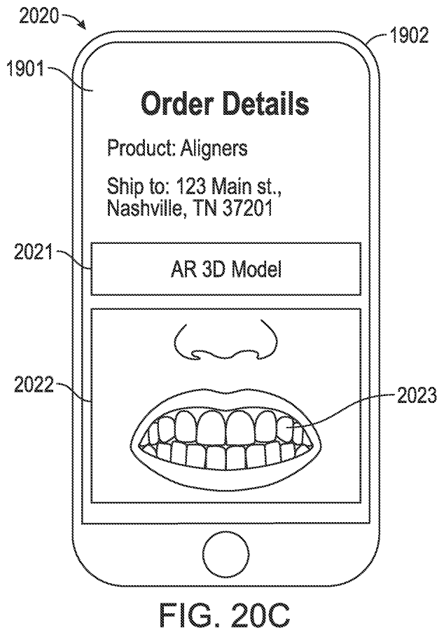

In some implementations, the MTP can also include providing content pages such as allowing a user to scan their teeth using user device 1902 (shown in FIG. 19C-19D). Further, additional content can be presented within content pages of the treatment plan interface 1901. In block 1738, a selection of sign in can include enabling a user to navigate to a portal or account dashboard. In block 1738, a selection of buy now can include validating and/or signing into an account, checking out (e.g., FIGS. 20A-20C), and navigating to an account dashboard. In another example, at block 1740 a treatment plan interface 1901 can be presented allowing a user to select from a plurality of actionable objects (described in detail with reference to FIGS. 19A-19I, 20A-20C, 21A-21J, and 23A-23G) such as, but not limited to, a sign in, or a buy now option. In block 1742, a selection of sign in can include enabling a user to navigate to a portal or account dashboard. In block 1742, a selection of buy now can include validating and/or signing into an account, checking out (e.g., FIGS. 20A-20C), and navigating to an account dashboard. In response to completing or navigating through blocks 1738 or 1742, the portal or account dashboard can be presented in block 1764. The account dashboard can be presented in a treatment plan interface 1901 and can include content such as, but not limited to, next steps for after an order is placed.

The treatment plan interface architecture 1700 is also shown to include a second flow starting at block 1712 where a user operating user device 1902 can begin a smile assessment (SA). In some implementations, an application of the user device 1902 may be configured to present an interface of the SA. For example, the application may be a web browser. Additionally, or alternatively, the application may be a mobile application. In various implementations, the application can include one or more application interfaces for presenting an application (e.g., mobile application, web-based application, virtual reality/augmented reality application, smart TV application and so on). At block 1714, the user can complete a smile assessment including selecting one or more actionable objects presented within the viewport of user device 1902 in response to questions about the user's current dentition, chief complaint, treatment preferences, and general goals for treatment. At block 1716, after the completion of the smile assessment the user can be presented with a call to action (CTA). In some implementations, at block 1718 the smile assessment can be performed on a computing system but the user may be invited to download the application to see their new smile (shown with reference to FIGS. 19A-19I, 20A-20C, 21A-21J, and 23A-23G). In various implementations, the user device 1902 may confirm the user has an account. If the user has an account, they may be asked via a presentation to sign in. If the user does not have an account, the user may be allowed to checkout. At block 1720, a selection of buy now can include validating and/or signing into an account and checking out (e.g., FIGS. 20A-20C). At block 1744, a new account can be created or if an account is already created, the user can be prompted to sign in. At block 1748, the user can then login to or access their account and account information where it can present shipping information and next steps associated with the treatment plan, which may include for example booking an appointment for an intraoral scan, purchasing an impression kit, or scheduling an in-person appointment with a dental practitioner to discuss next steps. At block 1750, when a user did not previously have an account, they can finish the setup process of setting up an account to access their account information.

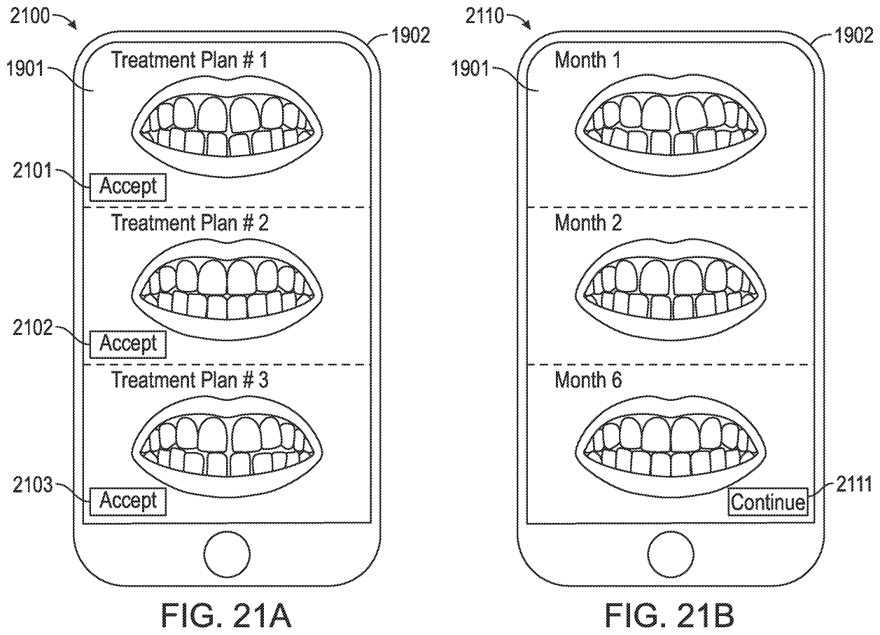

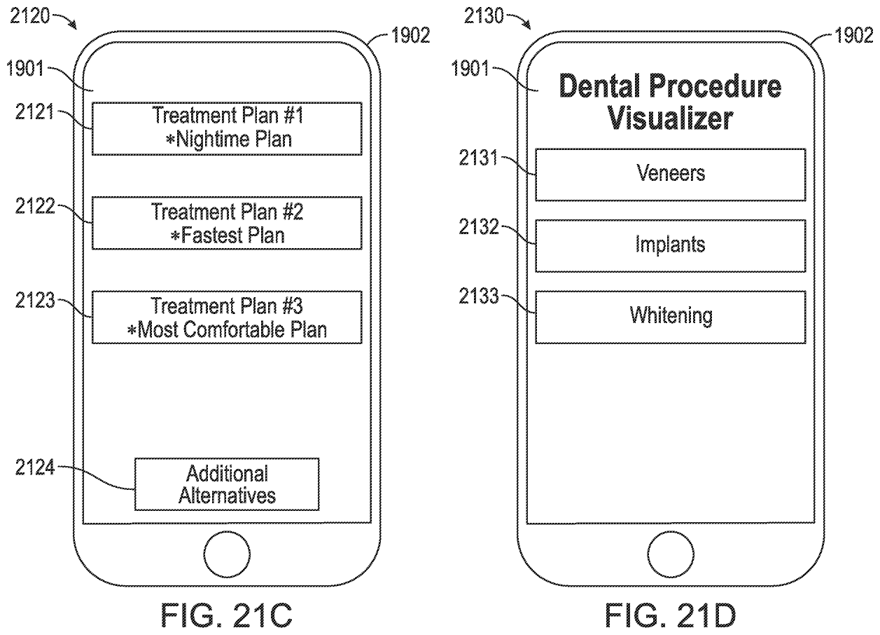

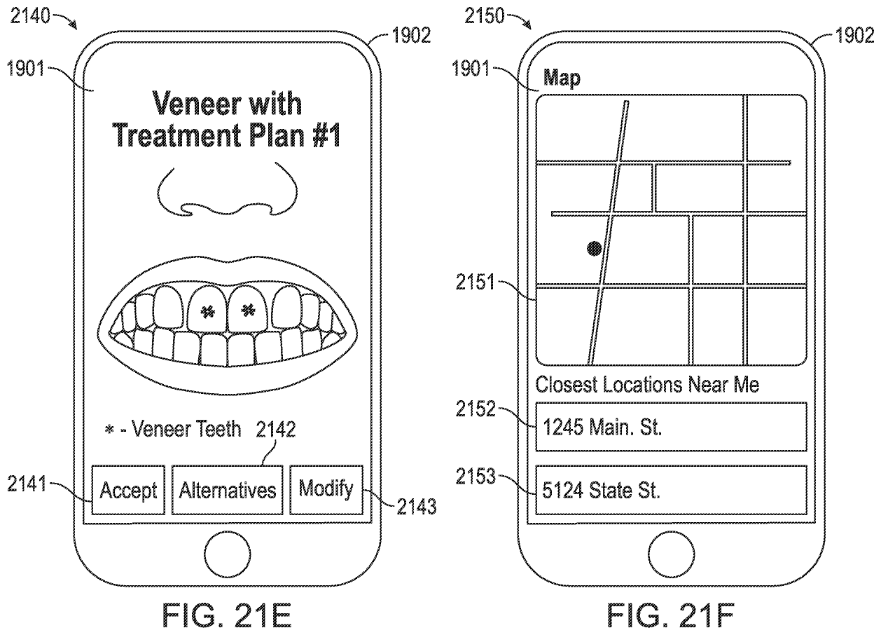

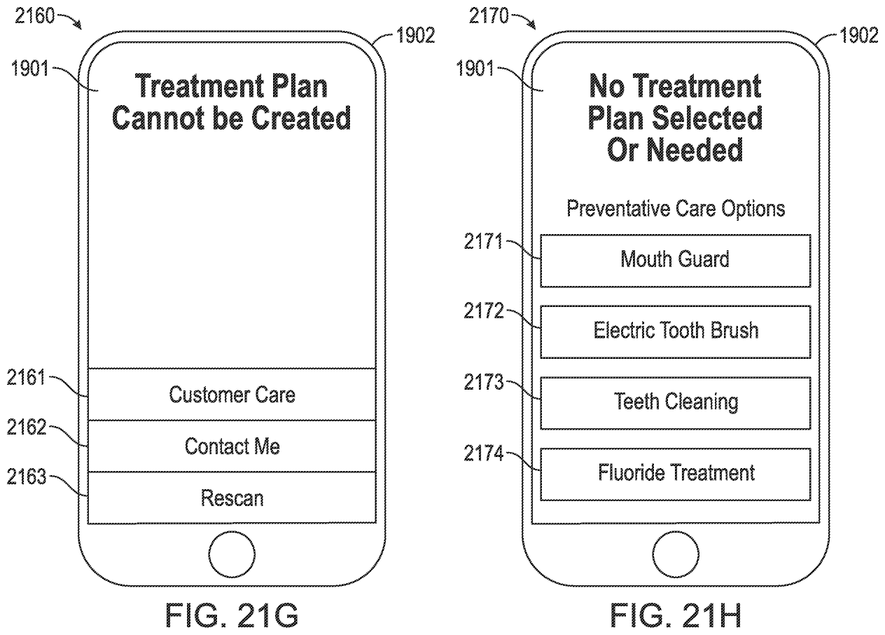

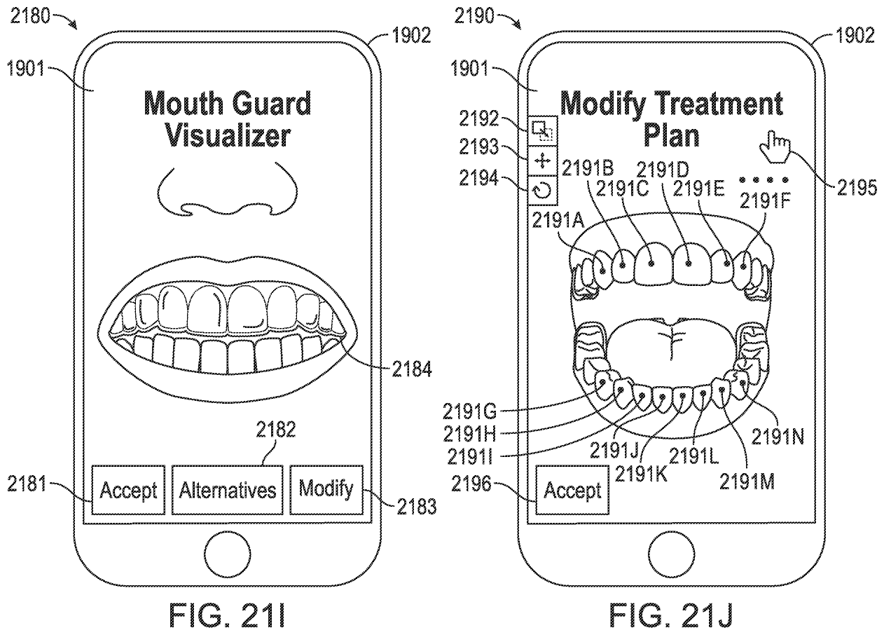

The treatment plan interface architecture 1700 is also shown to include a third flow starting at block 1724 where a user operating user device 1902 can begin a buy now process (e.g., 1905 and FIGS. 20A-20C). Additional details regarding buy now are described in detail with reference to FIGS. 20A-20C. It should be understood that the third flow, fourth flow, and fifth flow can be executed on an application. For example, the application may be a web browser. Additionally, or alternatively, the application may be a mobile application. In various implementations, the application can include one or more application interfaces for presenting an application (e.g., mobile application, web-based application, web browser application, virtual reality/augmented reality application, smart TV application and so on). As shown, the user can select a buy now interactive object to purchase a product such as aligners, dental arch, veneers, cavity products, etc. In particular, the user is provided options to purchase a product prior to performing an assessment, scan, or detection. The treatment plan interface architecture 1700 is also shown to include a fourth flow starting at block 1726 where a user operating user device 1902 can begin a sign-in process. As shown, the user can navigate to a login screen where they can enter a username and password or reset a password. Upon entering their account information, the user can be presented with an account dashboard (e.g., FIG. 19A). The treatment plan interface architecture 1700 is also shown to include a fifth flow starting at block 1732 where a user operating user device 1902 can begin a location lookup process (e.g., FIG. 21F). Additional details regarding location features are described in detail with reference to FIG. 21F. As shown, the user can navigate a location content page to identify locations of lab, dentists, etc., and determine contact information and addresses of the identified locations.

Additionally, block 1754 can include presenting an MTP viewer and modifier as shown with reference to FIG. 21J. The MTP viewer 1754 may present the user with a preliminary treatment plan, or an MTP, for visualization by the user. Block 1756 can include presenting a checkout as shown with reference to FIG. 20A. Block 1762 can include prompting the user to verify their account (e.g., two-stop authentication). At block 1758, a content page including order details that may include account setup information can be presented, for example, as shown with reference to FIG. 21C. At block 1760, after the order for a product is placed by the user, the treatment planning computing system 102 and other systems and devices herein can provide updates. For example, at 1760(1) the user can be sent an impression kit (IK) and/or can be presented with the option to book a scan at a lab or dental professional's office. At 1760(2), the user can complete medical history and consents via the treatment plan interface 1901. At 1760(3), once the IK is delivered or the scan or appointment is booked, the customer can take and upload additional images and videos (e.g., 2D or 3D) optionally using dental appliance 2405 (“Smile Stretcher”). At 1760(4), if the customer requested an impression kit (IK), the customer can mail back the impressions, or if the customer booked an appointment, the customer can obtain an intraoral scan. At 1760(5), the treatment planning process for creating the final treatment plan can be executed by the treatment planning computing system 102 including impression or intraoral scan approval, photo approval, final treatment plan creation, prescribing doctor review, modification if necessary, and approval of the final treatment plan, and sending the final treatment plan to the customer. At 1760(6), once the final treatment plan is created, the mobile treatment plan can be replaced in the treatment plan interface 1901 (sometimes referred to herein as “a TP viewer”). At 1760(7), the treatment planning computing system 102 may wait X amount of days (e.g., 3 days, 5 days, 7 days, 10, days, 14 days, etc.) before putting aligners or other products into production, which can be sent directly to the customer without first providing the aligners or other products to a dentist

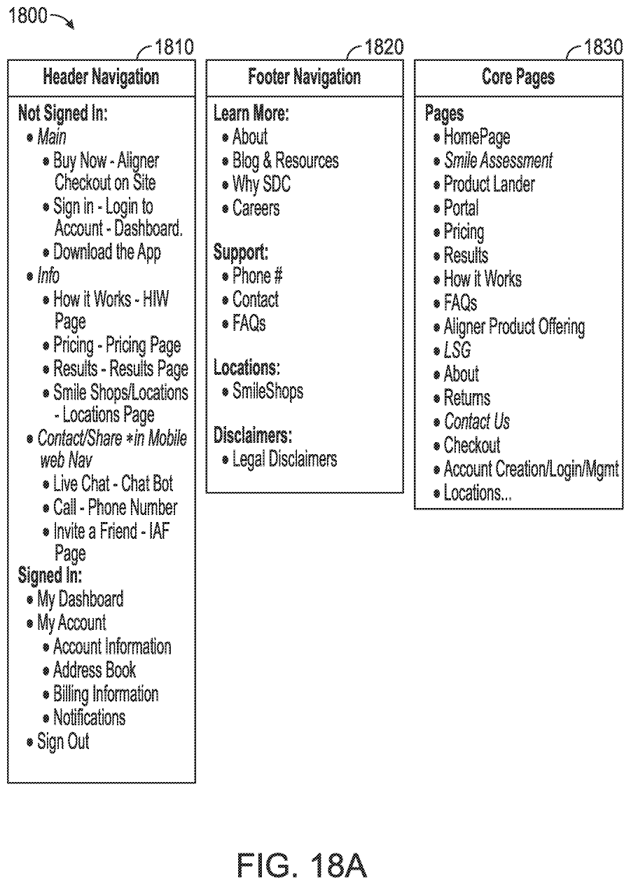

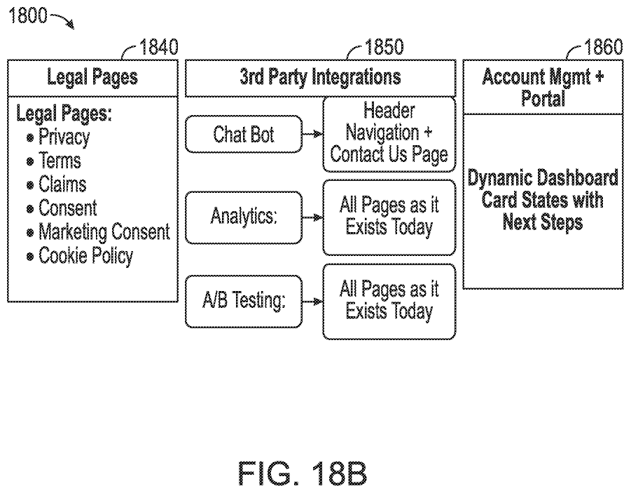

FIGS. 18A-18B are block diagrams depicting an example of a treatment plan interface architecture 1800, according to illustrative embodiments. The treatment plan interface architecture 1800 is shown to include example navigations, links, content, and objects within treatment plan interface 1901. For example, the treatment plan interface architecture 1800 can be executed and presented in a web-browser or mobile application and can include a header navigation 1810, a footer navigation 1820, a core page 1830, legal pages 1840, third party integrations 1850, and account management and portal 1860.

In general, FIGS. 19A-19I, 20A-20C, 21A-21J, and 23A-23G illustrate the treatment plan interface 1901 that can be rendered at the user device 1902 to provide interactable items, objects, and content associated with treatment plans related to dental health and hygiene. User device 1902 can include similar features and functionalities as user device 1302 as described with reference to FIGS. 13A-13D. The treatment plan interface can include a plurality of interfaces, items, objects, and content. For example, the user device 1902 can execute and provide the treatment plan interface 1901 with dental information, assessments, and visualizations, based on scans executed by user device 1302, visualization engine 1306, intake computing system 104, final position processing engine 210, and/or any other systems or device described herein. In some embodiments, a user may establish or have a user account with login credentials and account data stored in a database (e.g., memory 114). The account data can include information regarding the user devices registered with the treatment plan interface 1901.

In some implementations, each field may be a form of an input field (e.g., text input, buttons, camera input, drop-downs, speech-to-text, text-to-speech, toggle, pop-up, etc.) that can be selectable and actionable by a user. Furthermore, various additional fields are contemplated in this disclosure. In some implementations, once the user provides input to various fields in treatment plan interface 1901, the user device 1902 may send (e.g., over network 110) the input to treatment planning computing system 102 (or other systems or devices described herein) for storage and/or analysis. In some implementations, the user associated with a user account may be able to manage the user device 1902 preferences and user preferences associated with the user account. Management can include, but is not limited to, changing configurations (e.g., names, look and feel of treatment plan interface 1901, etc.), and/or user information (e.g., payment preferences, current treatment plans, geolocation, passwords, historical data, etc.).

Still referring to FIGS. 19A-19I, 20A-20C, 21A-21J, and 23A-23G generally, the user device 1902 (sometimes referred to herein as a “mobile device”) may be a mobile computing device, smartphone, tablet, smart headset, virtual reality (VR) headset, augmented reality (AR) headset, smart watch, smart sensor, or any other device configured to facilitate receiving, displaying, and interacting with content (e.g., web pages, mobile applications, etc.). As used herein, virtual reality, augmented reality, and mixed reality may each be used interchangeably to refer to any kind of extended reality, including virtual reality, augmented reality, and mixed reality.

User device 1902 may include an application to receive and display items, objects, and content and to receive user interaction with the items, objects, and content. For example, the application may be a web browser. Additionally, or alternatively, the application may be a mobile application associated with the treatment planning computing system 102. User device 1902 may also include an input/output circuit for communicating data over network 110 (e.g., receive and transmit to various devices and systems described herein, such as but not limited to, treatment planning computing system 102, intake computing system 104, fabrication computing system 106, image converter 1304, visualization engine 1306, etc.).

In various implementations, the application of user device 1902 interacts with a treatment planning computing system 102, visualization engine 1306, and other computing systems described herein to receive items, objects, and content associated with treatment plans related to dental health and hygiene. For example, the application of user device 1902 may receive the treatment plan interface 1901 and other data associated with the treatment plan interface 1901 such as, but not limited to, representations (2D or 3D) of the teeth of the patient who operates user device 1902. The treatment plan interface 1901 may include web-based content such as a web page or other online information. The treatment plan interface 1901 may include instructions (e.g., scripts, executable code, etc.) that when interpreted by the application causes the application to display a graphical user interface such as an interactable web page and/or an interactive mobile application to a user (e.g., treatment plan interface 1901). In various implementations, the application can include one or more application interfaces for presenting an application (e.g., mobile application, web-based application, virtual reality/augmented reality application, smart TV application, etc.).

Still referring to FIGS. 19A-19I, 20A-20C, 21A-21J, and 23A-23G generally, the treatment plan interface 1901 may include a collection of software development tools contained in a package (e.g., software development kit (SDK), application programming interface (API), integrated development environment (IDE), debugger, etc.). For example, the treatment plan interface 1901 may include an application programming interface (API). In another example, the treatment plan interface 1901 may include a debugger. In yet another example, the treatment plan interface 1901 may be an SDK that includes an API, a debugger, and IDE, and so on. In some implementations, the treatment plan interface 1901 includes one or more libraries having reusable functions that interface with a particular system software (e.g., iOS, Android, Linux, etc.). The treatment plan interface 1901 may facilitate embedding functionality into the user device 1902.

Accordingly, the ability for at-home users (e.g., patients) to utilize a treatment plan interface 1901 to review 3D digital models of their teeth, understand the health of their teeth, and modify configurations of treatment plans, provides at-home users the ability to view and perform actions on the treatment plan interface 1901 that typically only a treating dentist, technician, orthodontist, or administrator can view and perform and often only using expensive and highly complex software that is not generally available to the public. This treatment plan interface 1901 and computer architecture methodologies provide improved data presentation techniques, modification techniques, and computer architectures.

Furthermore, in many systems, the creation of treatment plans (e.g., initial or finalized) and discovery of dental diseases and conditions are based on images taken by dental impression or scanning equipment that are bulky, expensive, and/or not available for at-home use. However, the ability to modify aspects of the treatment plans, create treatment plans, and discover dental disease and conditions based on an at-home device such as a mobile phone, provides patients and dental professionals enhanced flexibility on how treatment plans are created and how their desired teeth will look in a remote setting, without necessitating costly and time-consuming in-person appointments. This improved approach allows treatment plan interfaces to produce significant improvements to the engagements presented through the viewport of the computing device and reduce errors with treatment plans associated with patient preferences. Therefore, aspects of the present disclosure address problems in application development by providing an improved interface tool for the production of treatment plans and dental disease and condition detection. Moreover, the present disclosure also provides improvements to traditional computing systems by providing a treatment planning interface that can enable changes to content on a graphical user interface based on the user selections of interactive objects. Moreover, this technical solution enables treatment planning interfaces to customize user experiences on an at-home device to improve engagement of users when presented through the viewport of the computing device. In particular, the customization enables the user devices and computing systems described herein to update 3D digital models of teeth based on preference parameters from user interactions with the treatment planning interface. As such, the preference parameters can be used in combination with the parameters typically used (e.g., by dental impression or scanning equipment) to generate a customized 3D digital model for a particular patient.

Referring now to FIGS. 19A-19I, example illustrations depicting a treatment plan interface 1901 are shown, according to illustrative embodiments. In general, the user device 1902 can present the treatment plan interface 1901 in the viewport (e.g., display 1308) of user device 1902 that can include one or more items, objects, and content. Treatment plan interface 1901 can be any type of application (e.g., in an application store, downloaded, custom, and so on) utilized by the user of user device 1902.

In example illustration 1900 (FIG. 19A), a plurality of graphical interface objects (sometime referred to herein as “graphical visualizations”) are displayed on the treatment plan interface 1901. The treatment plan interface 1901 is shown to include a mobile treatment plan dashboard including a plurality of unactionable objects (e.g., text and content) and actionable (or selectable) objects (e.g., items and objects) such as buttons 1903, 1904, 1905, 1906, 1907, 1908, and 1909. In this illustration, a user of the treatment plan interface 1901 can select one or more buttons to navigate to other content (e.g., content pages) of the treatment plan interface 1901. For example, button 1903 can be actionable (i.e., selectable) by the user and can be configured to, upon selection, text the user device 1902 a signup code or information regarding their account (e.g., current plans). In another example, button 1904 can be actionable by the user and can be configured to, upon selection, navigate to a smile assessment content page that can be presented within the viewport of user device 1902 and asks questions about the user's current dentition, chief complaint, treatment preferences, and general goals for treatment. In yet another example, button 1905 can be actionable by the user and can be configured to, upon selection, navigate to a buy now content page as shown with reference to example illustration 2010. In yet another example, button 1906 can be actionable by the user and can be configured to, upon selection, navigate to a sign in page for the user to enter account information (e.g., username and password) to sign in to their account. In yet another example, button 1907 can be actionable by the user and can be configured to, upon selection, navigate to an insurance page to enable a user to upload or enter insurance information. The insurance information can then be linked to the account of the user. In yet another example, button 1908 can be actionable by the user and can be configured to, upon selection, navigate to a map (as shown with reference to example illustration 2150) associated with physical locations of where patients (e.g., users) can set up a treatment plan, obtain an intraoral scan, schedule an in-person consultation or appointment, or otherwise gather or provide more information regarding treatment. In yet another example, button 1909 can be actionable by the user and can be configured to, upon selection, navigate to a mobile treatment plan content page as shown with reference to example illustration 1910.

In example illustration 1910 (FIG. 19B), a plurality of graphical interface objects are displayed on the treatment plan interface 1901. The treatment plan interface 1901 is shown to include a mobile treatment plan content page including a plurality of unactionable objects (e.g., text and content) and actionable (or selectable) objects (e.g., items and objects) such as buttons 1911, 1912, 1913, and 1914. In this illustration, a user of the treatment plan interface 1901 can select one or more buttons associated with an assessment such as a teeth alignment (e.g., 1911), a cavity detection (e.g., 1912), a periodontitis disease detection (e.g., 1913), and/or general dental condition detection (e.g., 1914). For example, button 1911 can be actionable (i.e., selectable) by the user and can be configured to, upon selection, navigate to a teeth alignment content page as shown with reference to example illustration 1920. In another embodiment, button 1911 can be actionable by the user and can be configured to, upon selection, navigate to a content page that gathers information from the user regarding the user's current teeth alignment, dental history, and the user's teeth alignment goals. In another example, button 1912 can be actionable by the user and can be configured to, upon selection, navigate to cavity detection content page as shown with reference to example illustration 1950. In some implementations, prior to the navigation to example illustration 1950, the user may capture images of the user's dentition using a camera or sensor (e.g., using a LIDAR sensor to capture objects and surface with a laser and measuring the time for the reflected light to return the receiver on the user device 1901, accelerometer, etc.) of user device 1901. In yet another example, buttons 1913 or 1914 can be actionable by the user and can be configured to, upon selection, navigate to other content pages for assessing periodontitis or other general dental conditions. An AI model can detect various diseases based on using the capture images and videos and output a probability of a disease or condition and can optionally connect the user with a dental practitioner if the probability of a disease or condition exceeds a determined threshold.

In example illustration 1920 (FIG. 19C), a plurality of graphical interface objects are displayed on the treatment plan interface 1901. The treatment plan interface 1901 is shown to include a teeth scanning content page including a plurality of unactionable objects (e.g., text and content) such as indicator 1921. In some implementations, in response to a selection of MTP Scan button 1909 of FIG. 19A, example illustration 1920 may be presented (skipping example illustration 1910). For example, instead of requesting the user to select buttons 1911-1914, the treatment plan interface 1901 may perform each of the functionality and features in one mobile treatment scan, in response to selecting MTP Scan button 1909. As shown, the user device 1902 can use one or more cameras and/or sensors to capture real-time images and videos of the plurality of teeth of the patient. For example, the user device 1902 can be configured to perform similar operations to those of scanning device(s) 214 such as capturing two-dimensional (2D) images and videos of a dentition of the patient (i.e., user). In another example, the user device 1902 can capture three-dimensional (3D) images and videos of the patient. In some implementations, the 2D and 3D images and/or videos (collectively referred to as “scans”) can be used as input to create a 3D digital model of the patient's dentition in real-time (e.g., as the dentition is scanned). The user device 1902 can be configured to provide the 2D and 3D images and/or videos to the intake system 104 and/or any other computing system or device described herein. An example of a 3D digital model is shown and described in detail with reference to FIG. 4 and FIG. 19F. The indicator 1921 can be presented to provide an indication to the user (e.g., using various colors given for various statuses) indicating the status of the teeth scanning (e.g., red indicating less than 50% is scanned, yellow indicating less than 100% is scanned, and green indicating the scanning is 100% complete) for generating the 3D digital model of the dentition of the patient. Additional examples and description of capturing images and videos representing at least a portion of a mouth of a user are described with further reference to U.S. patent application Ser. No. 17/581,811, filed Jan. 21, 2022, the entire disclosure of which is incorporated by reference herein.

In some implementations, a user using the user device 1902 may capture an image or video of the user using a camera. In some embodiments, the camera used to capture the image or video of the user may be a “front-facing” camera. For example, a front-facing camera may be a camera that is on a front of the user device (e.g., the same side as a display of a smartphone) such that the user may view the display of the user device 1902 while facing the camera. In other embodiments, the camera used to capture the image or video of the user may be a “rear-facing” camera. A rear-facing camera may be a camera that is on a back of the user device 1902 (e.g., the side opposite the display of a smartphone) such that the user may not be able to view the display of the user device and face the rear-facing camera at the same time. The image of the user captured by the camera (either the front-facing camera or the rear-facing camera) may be ingested by the user device 1902 using an input/output circuit. Additional examples and description of capturing images and videos representing at least a portion of a mouth of a user are described with further reference to U.S. patent application Ser. No. 17/581,811, filed Jan. 21, 2022, the entire disclosure of which is incorporated by reference herein.

In example illustration 1930 (FIG. 19D), a plurality of graphical interface objects are displayed on the treatment plan interface 1901. The treatment plan interface 1901 is shown to include a teeth scanning content page including a plurality of unactionable objects (e.g., notification). In some implementations, the treatment plan interface 1901 may present commands (e.g., textually, verbally, and/or another haptic feedback) to the user of the user device 1902. As shown, the command “attach wired camera” may be presented on the display of the user device 1902, and in response to the user attaching the camera (or another sensor), the treatment plan interface 1901 may present a real-time view of what the camera is capturing. In some implementations, the user device 1902 can capture images and videos of the dentition of the patient while the camera or sensor is moved around in the mouth or a partially closed volume. The captured images and videos (e.g., 2D and 3D) can be used as input to create a 3D digital model of the patient's dentition in real-time (e.g., as the dentition is scanned).

In example illustration 1940 (FIG. 19E), a plurality of graphical interface objects are displayed on the treatment plan interface 1901. The treatment plan interface 1901 is shown to include a treatment plan content page including a plurality of unactionable objects (e.g., mockup of potential dentition of the patient if a treatment plan is selected) and actionable (or selectable) objects (e.g., items and objects) such as buttons 1941, 1942, and 1943. In particular, the graphical interface objects can enable the user to visualize the potential dentition of themselves (i.e., a preliminary treatment plan). In various implementations, the visualization is 3D and can be manipulated by the user to see one or both arches from different angles and/or in different occlusal views (i.e., arches in contact with one another in a closed bite, and arches not in contact with one another). In some implementations, a 3D model can be created locally by the user device 1902 and presented in treatment plan interface 1901. In various implementations, the 2D and 3D scans can be sent to intake computing system 104 and/or treatment planning computing system 102 to create a 3D model. In some implementations, alternative treatment plans can be presented in treatment plan interface 1901, for example, as shown with reference to FIG. 21A. Additionally, stages of a treatment plan in relation to a specific time period in the future (e.g., month 1, month 2, month 6; or September, October, November) can be presented in treatment plan interface 1901, for example, as shown with reference to FIG. 21B. Also as shown, the treatment plan interface 1901 can provide one or more actionable buttons (e.g., 1941-1943). For example, button 1941 can be actionable (i.e., selectable) by the user and can be configured to, upon selection (e.g., from haptic feedback such as touch, voice, etc.), navigate to an order content page as shown with reference to example illustration 2000. In another example, button 1942 can be actionable by the user and can be configured to, upon selection, navigate to an alternative treatment plan content page as shown with reference to example illustration 2100. In yet another example, button 1943 can be actionable by the user and can be configured to, upon selection, enable the user to modify the mobile treatment plan shown in example illustration 1940 as shown with reference to a modification content page in example illustration 2190. In the following example, the user could drag (e.g., using the touchscreen of user device 1902) a tooth to modify a desired dentition. Furthermore, the user could verbally request, upon selecting button 1943, the incisors be spaced apart wider. In some implementations, the user can modify a mobile treatment plan to a desired look and feel using treatment plan interface 1901. The modification can include updating, by the user device 1902 or the intake computing system 104 communicated over network 110, parameters to create the treatment plan such as preference parameters (i.e., treatment parameters) of the patient. As shown, the information relating to the treatment plan used by the systems described herein may take into account (e.g., use as input) biomechanical and biological parameters, as well as preference parameters of the user relating to tooth positions and tooth movement, such as the amount and volume of tissue or bone remodeling, the rate of remodeling or the relating rate of tooth movement, all to ensure that the treatment plan is safe and effective. In some implementations, the example illustration 1940 can also include education information (e.g., as shown with reference to 23C, 23D, 23F, and 23G) to explain factors that go into treatment lengths such as biological factors. The educational information can be personalize based on the age, race, or other demographic of the patient.

In example illustration 1950 (FIG. 19F), a plurality of graphical interface objects are displayed on the treatment plan interface 1901. The treatment plan interface 1901 is shown to include a cavity detection content page including a plurality of unactionable objects (e.g., text and content) such as indicator 1951, and actionable (or selectable) objects (e.g., items and objects) such as buttons 1952, 1953, and 1954. In some implementations, in response to a user selecting button 1912 in FIG. 19B, the user device 1902 may scan the teeth (e.g., as shown in FIG. 19C), may optionally create a 3D digital model, and may analyze the 2D image or 3D digital model for any indicators (e.g., tooth decay, holes, spots or stains, bleeding, bad breath) of a cavity (e.g., pit and fissure cavity, smooth surface cavity, root cavity). In some implementations, the treatment plan interface 1901 may also present a list of questions associated with cavity detection (e.g., “Have you experienced pain in chewing?”, “Have you noticed any tooth sensitivity to hot or cold?”). As shown, the indicator 1951 may be presented on the treatment plan interface 1901 indicating the location of the cavity on the 3D digital model and a potential cavity treatment plan. Also as shown, the treatment plan interface 1901 can provide one or more actionable buttons (e.g., 1952-1954). For example, button 1952 can be actionable by the user and can be configured to, upon selection, perform a rescan of the dentition of the patient. In another example, button 1953 can be actionable by the user and can be configured to, upon selection, navigate to the order content page as shown with reference to example illustration 2000 (e.g., to order a cavity treatment) or to a location finder content page as shown with reference to example illustration 2150 (e.g., to schedule an appointment). In yet another example, button 1954 can be actionable by the user and can be configured to, upon selection, send a notification or message to the treatment planning computing system 102 that can in turn, schedule a call or communication from a treating dentist or a dental professional.

In example illustration 1960 (FIG. 19G), a plurality of graphical interface objects are displayed on the treatment plan interface 1901. The treatment plan interface 1901 is shown to include a teeth scanning content page including a plurality of unactionable objects (e.g., text and content) such as indicator 1961. The indicator 1961 can be presented to provide an indication to the user (e.g., using various colors given for various statuses) indicating the status of the teeth scanning (e.g., red indicating the user has not bit down correctly or all the way, and green indicates the bite down is correct) for generating the 3D digital model of the dentition of the patient.

In example illustration 1970 (FIG. 19H), a plurality of graphical interface objects are displayed on the treatment plan interface 1901. The treatment plan interface 1901 is shown to include a disease detection content page including a plurality of unactionable objects (e.g., text and content) and actionable (or selectable) objects (e.g., items and objects) such as buttons 1971, 1972, and 1973. In some implementations, in response to a user selecting button 1913 or 1914 in FIG. 19B, the user device 1902 may scan the teeth (e.g., as shown in FIG. 19C), may optionally create a 3D digital model, and may analyze the 2D image or the 3D digital model for any indicators (e.g., swollen or puffy gums, bright red gums, receding gums, bad breath) of periodontitis (e.g., early, mild, moderation, advanced) or other general dental conditions (e.g., gingivitis, dry mouth, misalignment of dentition or teeth). Also as shown, the treatment plan interface 1901 can provide one or more actionable buttons (e.g., 1971-1973). For example, button 1971 can be actionable by the user and can be configured to, upon selection, perform a rescan of the dentition of the patient. In another example, button 1972 can be actionable by the user and can be configured to, upon selection, navigate to the order content page as shown with reference to example illustration 2000 (e.g., order a product or service associated with the identified disease or condition) or to a location finder content page as shown with reference to example illustration 2150 (e.g., to schedule an appointment). In yet another example, button 1973 can be actionable by the user and can be configured to, upon selection, send a notification or message to the treatment planning computing system 102 that can in turn, schedule a call or communication from a treating dentist or a dental professional.

In example illustration 1980 (FIG. 19I), a plurality of graphical interface objects are displayed on the treatment plan interface 1901. The treatment plan interface 1901 is shown to include a communication content page including a plurality of unactionable objects (e.g., text and content) and actionable (or selectable) objects (e.g., items and objects) such as buttons 1981, 1982, 1983, and 1984. Each of the buttons 1981-1984 can be actionable by the user and can be configured to, upon selection, send perform an action such as provide additional details because the user is confused (e.g., 1981), send a 3D model to the user (e.g., 1982, digitally (e.g., such as email, hyperlink, posting on account)), setup an in-person assessment (e.g., 1983), setup a phone call (e.g., 1984), or establish a chat with a chat bot or person (e.g., 1985).

Referring now to FIGS. 20A-20C, example illustrations depicting a treatment plan interface 1901, according to illustrative embodiments. The example illustrations of FIGS. 20A-20C include similar features and functionality as described with reference to FIGS. 19A-19I. For example, the user device 1902 can present the treatment plan interface 1901 in the viewport (e.g., display 1308) of user device 1902 which can include one or more items, objects, and content. Treatment plan interface 1901 can be any type of application (e.g., in an application store, downloaded, custom, and so on) utilized by the user of user device 1902.

In example illustration 2000 (FIG. 20A), a plurality of graphical interface objects are displayed on the treatment plan interface 1901. The treatment plan interface 1901 is shown to include an order content page including a plurality of unactionable objects (e.g., text and content) and actionable (or selectable) objects (e.g., items and objects) such as buttons 2003, 2004, and 2005. In some implementations, in response to a user selecting button 1941, 1953, or 1972, the treatment plan interface 1901 may present order details including automatically adjusting the total based on discounts (e.g., such as insurance information or promotion codes). For example, the user may provide insurance information and parameters in response to selecting button 1907 of FIG. 19A. As shown, the product, price, discount, and total can be presented on the treatment plan interface 1901. In some implementations, the product price can be adjusted based on various factors such as case severity, customer-requested modifications, product type, locations, insurance type, shipping distance, historical interactions, etc. Also as shown, the treatment plan interface 1901 can provide one or more actionable buttons (e.g., 2003-2005). For example, button 2003 can be actionable by the user and can be configured to, upon selection, offer additional items for purchase (e.g., aligner cleaner, aligner case, toothbrush, extended warranty, monthly subscription). In another example, button 2004 can be actionable by the user and can be configured to, upon selection, navigate to mobile treatment plan content page (e.g., illustration 1910) to offer a user to perform additional assessments and detections. In another example, button 2005 can be actionable by the user and can be configured to, upon selection, navigate to a checkout content page as shown with reference to example illustration 2010.

In example illustration 2010 (FIG. 20B), a plurality of graphical interface objects are displayed on the treatment plan interface 1901. The treatment plan interface 1901 is shown to include a checkout content page including a plurality of unactionable objects (e.g., text and content) and actionable (or selectable) objects (e.g., items and objects) such as text input fields 2011, 2012, 2013, and 2014 and buttons 2015 and 2016. In some implementations, in response to a user selecting button 2005, the treatment plan interface 1901 may present a checkout content page including actionable fields for input payment information such as, but not limited to shipping information (e.g., 2011) and credit card information (e.g., 2012-2014, or other payment information such a debit card information, cryptocurrency account information, etc.). Also as shown, the treatment plan interface 1901 can provide one or more actionable buttons (e.g., 2015-2016). For example, button 2015 can be actionable by the user and can be configured to, upon selection, navigate to an order content page as shown with reference to example illustration 2020. In another example, button 2016 can be actionable by the user and can be configured to, upon selection, navigate to the order content page as shown with reference to example illustration 2022. In various implementations, the buy now, pay later button 2015 may present, prior to navigating to the order content page an amortization schedule of the periodic payment amounts, interest rate, total number of payments, and amount of principal. In some implementations, while buttons 2015 and 2016 can navigate to the order content page, the user device 1902 can provide payment details including the selection to the treatment planning computing system and/or order/purchase terminal 111 for processing (e.g., process immediately or based on a schedule).

In example illustration 2020 (FIG. 20C), a plurality of graphical interface objects are displayed on the treatment plan interface 1901. The treatment plan interface 1901 is shown to include an order content page including a plurality of unactionable objects (e.g., text and content) and actionable (or selectable) objects (e.g., items and objects) such as buttons 2021. In some implementations, in response to a user selecting button 2015 or 2016, the treatment plan interface 1901 may present order details from the order just placed including, but not limited to, the product and shipping address. As shown, an AR 3D model 2022 can be presented on the display and can be superimposed, over the teeth, visual indicia 2023 of the ordered product, such as how the dentition of the patient could look after the product is used for a specified period of time. To expand the AR 3D model, the user can select button 2021 to go full-screen enabling the user to review and perceive the visual indicia 2023 on the entire display of user device 1902. Additionally, to present the AR 3D model 2022, the user device 1902 or another system described herein can generate a 3D digital model of the teeth and in real-time continuously collect environmental data (e.g., capturing images and videos of the patient), and can automatically update in real-time (or near real-time) the visual indicia 2023 depicted over the teeth of the patient. In various embodiments, environmental data can include, but is not limited to, orientation data of the user device 1902 (e.g., vectors, axes, planes, positions, etc.), orientation data of a portion of a body (e.g., transverse plane, coronal plane, sagittal plane, associated with anatomy of the portion of the body), room/area information, types of electronic devices, user information, etc.

Referring now to FIGS. 21A-21J, example illustrations depicting a treatment plan interface 1901, according to illustrative embodiments. The example illustrations of FIGS. 21A-21J include similar features and functionality as described with reference to FIGS. 19A-19I and 20A-20C. For example, the user device 1902 can present the treatment plan interface 1901 in the viewport of user device 1902 which can include one or more items, objects, and content. Treatment plan interface 1901 can be any type of application (e.g., in an application store, downloaded, custom, and so on) utilized by the user of user device 1902.

In example illustration 2100 (FIG. 21A), a plurality of graphical interface objects are displayed on the treatment plan interface 1901. The treatment plan interface 1901 is shown to include an alternative treatment plan content page including a plurality of unactionable objects (e.g., mockup of potential dentitions of the patient if a treatment plan is selected) and actionable (or selectable) objects (e.g., items and objects) such as buttons 2101, 2102, and 2103. In particular, the graphical interface objects can enable the user to visualize a plurality of potential dentitions of themselves and select (e.g., via a button) a desired treatment plan based on the user's preferences. In some implementations, a 3D model can be created locally by the user device 1902 and presented in treatment plan interface 1901. In various implementations, the 2D and 3D scans can be sent to intake computing system 104 and/or treatment planning computing system 102 to create a 3D model. For example, in response to scanning the teeth (shown with reference to FIGS. 19C and 19D), the user can be presented with a single treatment plan (shown with reference to FIG. 19E) or a plurality of treatment plans based on preferences of the user or after a determination by the treatment planning computing system 102 or user device 1902 that more than one treatment plan can be generated. As shown, the plurality of treatment plans can be offered to fix some or all of the malocclusion of the teeth of the patient. For example, the patient may prefer to keep their diastema if they like their gap. In another example, the patient may prefer to keep their arch shape or opt to not correct more complex issues. In some implementations, the user could drag (e.g., using the touchscreen of user device 1902) a tooth to modify a desired dentition of one or more of the treatment plans (as shown with reference to the modification content page in example illustration 2190). In various implementations, the user could be presented with one treatment plan using 22-hour wear aligners and another treatment plan using limited-wear (e.g., nighttime-only wear) aligners. Furthermore, the user could verbally request that the canines or molars be spaced apart wider or spaced closer. In some implementations, the user can modify a mobile treatment plan to a desired look and feel using treatment plan interface 1901. Also as shown, the treatment plan interface 1901 can provide one or more actionable buttons (e.g., 2101-2103). For example, buttons 2101-2103 can be actionable by the user and can be configured to, upon selection, navigate to an order content page as shown with reference to example illustration 2000. In some examples, the only actionable object may be a continue button (as shown with reference to button 2111) that can navigate to a treatment selection content page shown in example illustration 2120. In some implementations, the viewport of the user device 1902 can be actionable by the user and can be configured to, upon selection, zoom in or out (e.g., by pinching with the figure) of the 3D digital model.

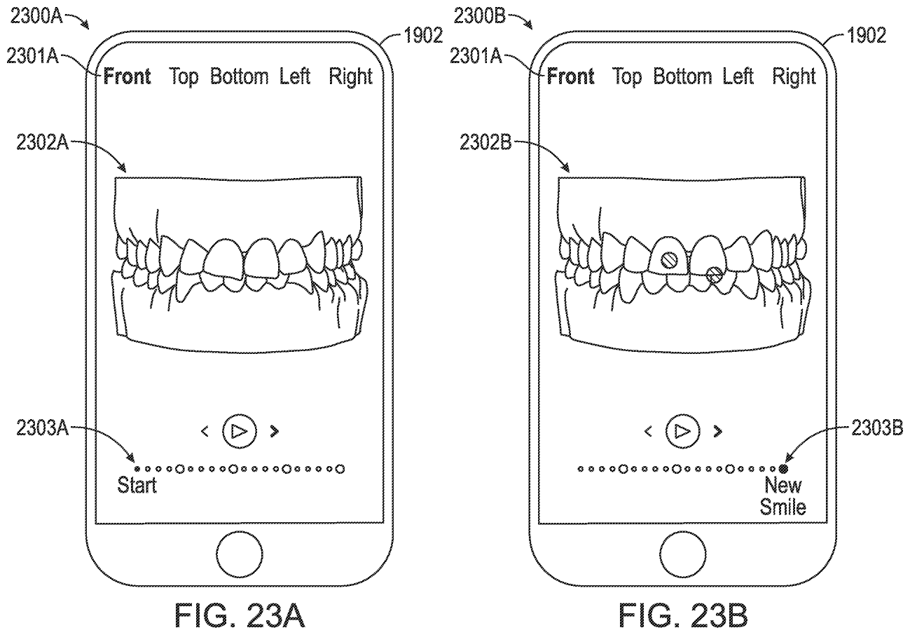

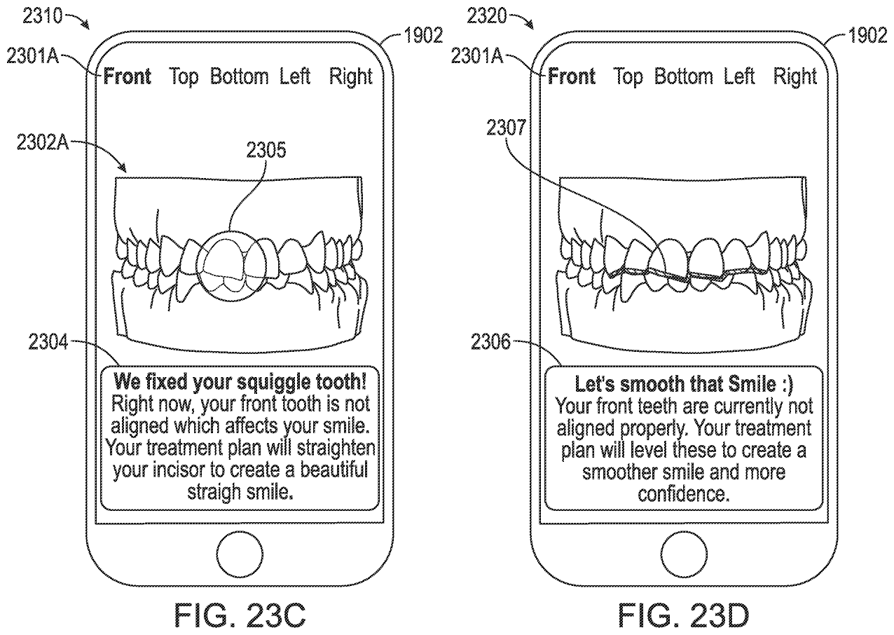

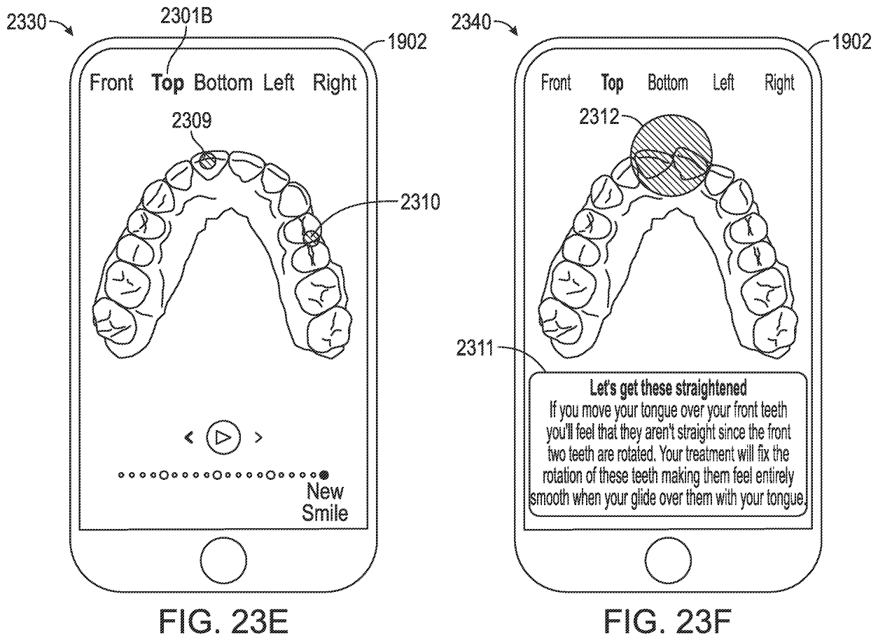

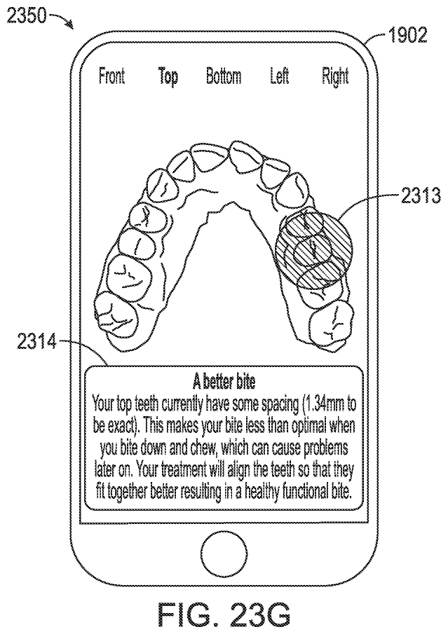

In example illustration 2110 (FIG. 21B), a plurality of graphical interface objects are displayed on the treatment plan interface 1901. The treatment plan interface 1901 is shown to include a mobile treatment plan content page including a plurality of unactionable objects (e.g., mockup of potential dentitions of the patient over time) and actionable (or selectable) objects (e.g., items and objects) such as button 2111. Button 2111 may optionally be a slider bar that allows the user to visualize treatment over the passage of time (e.g., in months) or over a plurality of stages of treatment (as shown with reference to slider 2303A and 2303B of FIGS. 23A-23B).

In particular, the graphical interface objects can enable the user to visualize a plurality of stages of dentition of themselves, after selection of buttons 1941, 1953, 1972, and/or 2101-2103, and prior to navigating to an order content page as shown with reference to FIG. 20A. In some implementations, the time periods (e.g., one month, two months, six months; or September, October, November) can be based on a user preference such as a key event the user is planning to attend (e.g., prom, wedding, reunion, speech). The treatment planning computing system 102 and/or user device 1902 can calculate periods of time based on when the product (e.g., aligners) will arrive after manufacturing and shipping. Also as shown, the treatment plan interface 1901 can provide one or more actionable buttons (e.g., 2111). For example, button 2111 can be actionable by the user and can be configured to, upon selection, navigate to an order content page as shown with reference to example illustration 2000.

In example illustration 2120 (FIG. 21C), a plurality of graphical interface objects are displayed on the treatment plan interface 1901. The treatment plan interface 1901 is shown to include a treatment selection content page including a plurality of unactionable objects (e.g., text and content) and actionable (or selectable) objects (e.g., items and objects) such as buttons 2121, 2122, 2123, and 2124. As shown, a plurality of treatment plans can be offered based on one or more preferences such as, but is not limited to, fastest plan (e.g., to correct the dentition of the patient in the quickest safe manner), nighttime only plan (e.g., wear aligners at night only), daily plans, comfortable plan (e.g., slower but more conformable plan to correct the dentition of the patient). As such, the user can customize the treatment plan based on various factors such as a scalloped edge or straight edge, the wear schedule, aligner color, aligner logo, addition of other designs on the aligner(s), arch shape, keeping gaps, correction up to a certain number of stages (e.g., 5 or 10 sets of aligners) to for example remain under a certain price point, etc. Also as shown, the treatment plan interface 1901 can provide one or more actionable buttons (e.g., 2121-2124). For example, buttons 2121-2123 can be actionable by the user and can be configured to, upon selection, navigate to an order content page as shown with reference to example illustration 2000. In another example, button 2124 can be actionable by the user and can be configured to, upon selection, navigate to an alternative treatment plan content page as shown with reference to example illustration 2100.

In example illustration 2130 (FIG. 21D), a plurality of graphical interface objects are displayed on the treatment plan interface 1901. The treatment plan interface 1901 is shown to include a visualizer content page including a plurality of unactionable objects (e.g., text and content) and actionable (or selectable) objects (e.g., items and objects) such as buttons 2131, 2132, and 2133. In this illustration, a user of the treatment plan interface 1901 can select one or more buttons associated with a visualization such as veneers (e.g., 2131), implants (e.g., 2132), and/or whitening (e.g., 2133). For example, buttons 2131-2133 can be actionable by the user and can be configured to, upon selection, navigate to a visualizer content page as shown with reference to example illustration 2140.

In example illustration 2140 (FIG. 21E), a plurality of graphical interface objects are displayed on the treatment plan interface 1901. The treatment plan interface 1901 is shown to include a visualizer content page including a plurality of unactionable objects (e.g., text and content) and actionable (or selectable) objects (e.g., items and objects) such as buttons 2141, 2142, and 2143. In particular, the graphical interface objects can enable the user to visualize the potential dentition of themselves after a procedure (and sometimes, as shown, in combination with a selected treatment plan). For example, the veneered teeth can be presented with an indicator on them. Also as shown, the treatment plan interface 1901 can provide one or more actionable buttons (e.g., 2141-2143). For example, button 2141 can be actionable by the user and can be configured to, upon selection (e.g., from haptic feedback), navigate to an order content page as shown with reference to example illustration 2000. In another example, button 2142 can be actionable by the user and can be configured to, upon selection, navigate to an alternative treatment plan content page (e.g., depicting if all the incisors were veneered). In yet another example, button 2143 can be actionable by the user and can be configured to, upon selection, enable to user to modify the visualized veneer treatment plan and mobile treatment plan shown in example illustration 2140 or example illustration 2190. In the following example, the user could select (e.g., using the touchscreen of user device 1902) a veneer to remove or add. In some implementations, the user can modify the treatment plans (e.g., veneer, mock, cavity, etc.) to a desired look and feel using treatment plan interface 1901.

In example illustration 2150 (FIG. 21F), a plurality of graphical interface objects are displayed on the treatment plan interface 1901. The treatment plan interface 1901 is shown to include a navigation content page including a plurality of unactionable objects (e.g., text and content) and actionable (or selectable) objects (e.g., items and objects) such as a map 2151 and buttons 2151 and 2153. In particular, the graphical interface objects can enable the user to visualize locations associated with physical locations on map 2151 where patients (e.g., users) can set up a treatment plan or perform dental operations and analysis. For example, if the user desires same day aligners the user could travel directly to a local dental lab to receive them directly from the manufacturer. In another example, the user may desire to schedule an in-person assessment with a practitioner after being presented with a mobile treatment plan. In this example, map 2151 may present the closest location for an appointment based on the geolocation of the user device 1902. Also as shown, the treatment plan interface 1901 can provide one or more actionable buttons (e.g., 2152-2153). For example, buttons 2152-2153 can be actionable by the user and can be configured to, upon selection, pop-up information about the selected location.

In example illustration 2160 (FIG. 21G), a plurality of graphical interface objects are displayed on the treatment plan interface 1901. The treatment plan interface 1901 is shown to include an error content page including a plurality of unactionable objects (e.g., text and content) and actionable (or selectable) objects (e.g., items and objects) such as buttons 2161, 2162, and 2163. In particular, the graphical interface objects can be presented in response to the user device 1902 or another system described herein (e.g., visualization engine 1306, intake computing system 104, final position processing engine 210), an error in generating a 3D digital model and/or creating a treatment plan (e.g., mock, veneer, cavity). For example, an error may occur if the dentition of the patient exceeds treatment parameters, a mobile treatment plan is beyond the bounds of a given confidence interval, there are dental contraindications that require clearance, etc. In the following example, the treatment plan interface 1901 can route the user for help (e.g., customer care, contact me) based on a selection of action buttons (e.g., 2161-2162). In another example, button 2163 can be actionable by the user and can be configured to, upon selection, perform a rescan of the dentition of the patient and determine a potential treatment.

In example illustration 2170 (FIG. 21H), a plurality of graphical interface objects are displayed on the treatment plan interface 1901. The treatment plan interface 1901 is shown to include a preventative care content page including a plurality of unactionable objects (e.g., text and content) and actionable (or selectable) objects (e.g., items and objects) such as buttons 2171, 2172, 2173, and 2174. In this illustration, a user of the treatment plan interface 1901 can select one or more buttons associated with preventative care options such a mouth guard (e.g., 2171), retainer, an electric toothbrush (e.g., 2172), teeth cleaning (e.g., 2173), and/or fluoride treatment (e.g., 2174). For example, buttons 2171-2174 can be actionable by the user and can be configured to, upon selection, navigate to a visualizer content page as shown with reference to example illustration 2170. In another example, button 2172 or 2713 can be actionable (i.e., selectable) by the user and can be configured to, upon selection (e.g., from haptic feedback), navigate to an order content page as shown with reference to example illustration 2000.

In example illustration 2180 (FIG. 21I), a plurality of graphical interface objects are displayed on the treatment plan interface 1901. The treatment plan interface 1901 is shown to include a visualizer content page including a plurality of unactionable objects (e.g., text and content) and actionable (or selectable) objects (e.g., items and objects) such as buttons 2181, 2182, and 2183. In particular, the graphical interface objects can enable the user to visualize the preventative care options (and sometimes in combination with a selected treatment plan). For example, the mouth guard (e.g., AR 3D model) can be presented on the display and can be superimposed over the teeth, visual indicia 2184, such as how the mouth guard of the patient would look. In another example, a retainer can be presented on the display and can be present via visual indicia 2184. Also as shown, the treatment plan interface 1901 can provide one or more actionable buttons (e.g., 2181-2183). For example, button 2181 can be actionable by the user and can be configured to, upon selection (e.g., from haptic feedback), navigate to an order content page as shown with reference to example illustration 2000. In another example, button 2182 can be actionable by the user and can be configured to, upon selection, navigate to an alternative treatment plan content page (e.g., depicting a bulkier mouth guard). In yet another example, button 2183 can be actionable by the user and can be configured to, upon selection, enable to user to modify the visualized mouth guard shown in example illustration 2180 (also as shown with reference to example illustration 2190).

In example illustration 2190 (FIG. 21J), a plurality of graphical interface objects are displayed on the treatment plan interface 1901. The treatment plan interface 1901 is shown to include a modification content page including a plurality of unactionable objects (e.g., text and content) and actionable (or selectable) objects (e.g., items and objects) such as selection areas 2191A, 2191B, 2191C (to 2191X), buttons 2192, 2193, 2194, 2196, and pointer 2195. In particular, the graphical interface objects can enable the user to visualize and modify (via haptic feedback or voice) the treatment plan (e.g., 3D digital model) of the user. In general, a modification can include generating a preference parameter for creating the 3D digital model and transmitting or inputting the preference parameter and the other parameters to generate a new 3D digital model incorporating the preference parameter. For example, a modification of the treatment plan can be performed by selecting button 2193 and dragging selection area 2191B to a different position on the treatment plan interface 1901 (e.g., left or right). Here, the preference parameter may include a key value pair of the tooth number and the offset (e.g., key: tooth 7, offset (value): −1, 0, 0), where the offset may be a value of x, y, z coordinates and the differences between the original treatment plan coordinates of the tooth and new modified coordinates of the tooth. In some implementations, the value may be another distinguishable number that can be interpreted by the user device 1902 or other systems described herein to make a modification. In another example, a modification of the treatment plan can be performed by selecting button 2191 and selecting selection area 2191A and then selecting a new position on the treatment plan interface 1901. Here, the preference parameter may include a coordinate in 2D- or 3D-space associated with a particular tooth. In yet another example, a modification of the treatment plan can be performed by selecting selection area 2191C and then selecting button 2194 to rotate the tooth slightly (e.g., 0.5 degrees, 1 degree, etc.). Here, the preference parameter may include an angle offset of a tooth (e.g., key: tooth 12, angle offset (value): 1.5 degrees). Accordingly, buttons 2196 can be actionable by the user and can be configured to, upon selection, submit or lock-in the modifications made by the user including storing and/or transmitting the preference parameters to create a new 3D digital model of the dentition of the patient. Therefore, the treatment plan interface 1901 enables a user to customize their desired treatment plan based on modifying 3D digital models in real-time, thereby providing improved graphical user interfaces. In particular, the user can modify the look and feel of their teeth, the position of the edge aligner, how fast or slow the treatment occurs, and so on, all within predetermined clinical limits to ensure safety and efficacy.

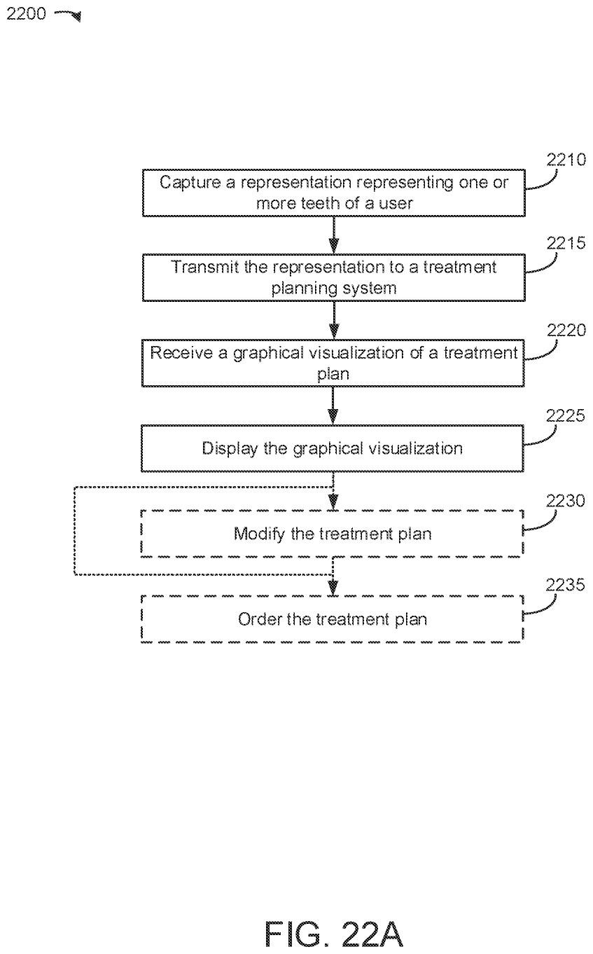

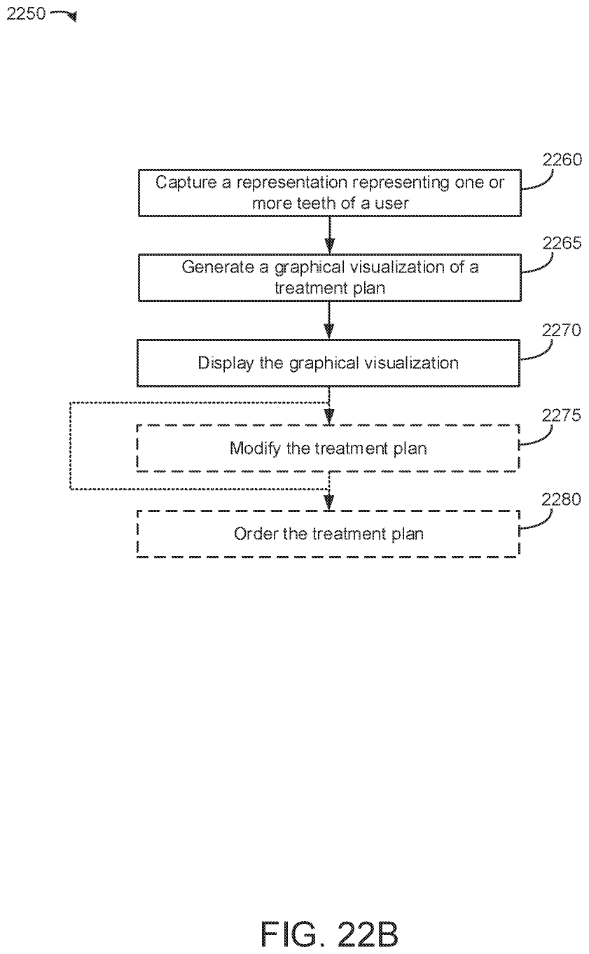

Referring now to FIGS. 22A-22B, flowcharts for methods 2200 and 2250 of visualizing a treatment of teeth, according to some embodiments. User device 1902 can be configured to perform methods 2200 and 2250. In particular, methods 2200 and 2250 can be presented and executed by treatment plan interface 1901. In broad overview of method 2200, at step 2210, the one or more processing circuits (e.g., user device 1902 in FIG. 19A) can capture a representation representing one or more teeth of a user. At step 2215, the one or more processing circuits can transmit the representation to a treatment planning system. At step 2220, the one or more processing circuits receive a graphical visualization of a treatment plan. At step 2225, the one or more processing circuits can display the graphical visualization. In some implementations, at step 2230, the one or more processing circuits can modify the treatment plan. In some implementations, at step 2235, the one or more processing circuits can order the treatment plan. Additional, fewer, or different operations may be performed depending on the particular arrangement. In some embodiments, some or all operations of method 1600 may be performed by one or more processors executing on one or more computing devices, systems, or servers. In various embodiments, each operation may be reordered, added, removed, or repeated.

Referring to method 2200 in more detail, at step 2210 the one or more processing circuits can capture a representation representing one or more teeth of a user via a camera of a user device. In some implementations, the representation includes a plurality of images (or videos) of a user's dentition, where the plurality of images are from specific angles. In some implementations, the one or more processing circuits can present a treatment plan interface (e.g., 1901) on a viewport of the one or more processing circuits. The user can navigate the treatment plan interface 1901 by making selections of actionable objects (as shown with reference to FIGS. 19A-19I, 20A-20C, 21A-21J, and 23A-23G). For example, the user may select button 1909 (e.g., mobile treatment plan) which can guide the user through capturing representations representing one or more teeth of the user (as shown with reference to FIGS. 19B, 19C, 19D, and 19G)

At step 2215, the one or more processing circuits can transmit the representation to a treatment planning system (e.g., 102). The representation can include parameter data and the images or videos captured by the one or more processing circuits. The representations can be a combination of 2D and 3D content (e.g., images, videos, etc.). While the one or more processing circuits wait for a response from the treatment planning system (or wait for the one or more processing circuits to generate a graphical visualization as described in method 2250), the user can be presented with additional interactable content such as the graphical visualizations disclosed in FIGS. 20A-20C, 21D, 21F, 21H, and 21I.

At step 2220, the one or more processing circuits can receive a graphical visualization of a treatment plan for moving the one or more teeth of the user from the treatment planning system, the graphical visualization (e.g., 19E, 21J) generated based on the representation. In some implementations, the graphical visualization further includes at least one of a view of one or more stages of the treatment plan (e.g., 21B), a view of one or more alternative treatment plans (e.g., 21A, 21E), an interactive tool for modifying the treatment plan (e.g., 21J), or a checkout option (e.g., 20A-20C). In some implementations, the graphical visualization can be treatment plans for other dental conditions or a combination of dental conditions such as cavities (e.g., 19F) or veneers (e.g., 21E). As shown, the graphical visualization can be a 3D digital model of a potential dentition (or dentitions) of the user. In various implementations, the graphical visualization can be accessible via a user portal accessible via log-in credentials and where the display further includes visual indicia based on the treatment plan superimposed over the one or more teeth of the user based on the treatment plan (e.g., FIG. 20C or FIG. 21I). Additionally, the graphical visualizations can include actionable content such as disclosed in FIGS. 23A-23G. As shown with reference to FIGS. 23A-23G, the user can review a preliminary treatment plan mockup (e.g., slide and rotate using haptic feedback).

At step 2225, the one or more processing circuits can display the graphical visualization. In some implementations, the graphical visualization can include a 3D representation corresponding to the one or more teeth of the user and an interactive object associated with the treatment plan. The interactive object can be actionable objects (or objects) configured to enable a selection of the treatment plan (or other selections) by the one or more processing circuits. For example, a selection can include, but not be limited to, purchasing the treatment plan, ordering the treatment plan, or a selection of a preferred treatment plan from among two or more treatment plans. In some implementations, the graphical visualization comprises a planned final position of the one or more teeth after the user has completed the treatment plan (e.g., FIG. 19E and FIG. 20C).

At step 2230, the one or more processing circuits can modify the treatment plan such as by receiving a user input to adjust the treatment plan. It should be understood that the dotted lines around step 2230 indicate the step may or may not be performed in combination with steps 2210-2225. For example, the one or more processing circuits can receive a user input to adjust the treatment plan and in turn, transmit to the treatment planning system a request to adjust the treatment plan based on receiving the user input. In some implementations, adjusting the treatment plan comprises adjusting the one or more teeth in the graphical visualization (e.g., FIG. 21J), wherein adjusting the one or more teeth further includes updating at least one parameter of the graphical visualization. Accordingly, the 3D digital model can be modified (e.g., 21A, 21C, 21J) and in response, the one or more processing circuits can receive a second graphical visualization of a second treatment plan based on the adjustment to the treatment plan and then display the second graphical visualization. The second graphical visualization can include a second 3D representation corresponding to the one or more teeth of the user and a second interactive object associated with the second treatment plan.

At step 2235, the one or more processing circuits can order or purchase the treatment plan such as by selecting a button on the graphical visualization. It should be understood that the dotted lines around step 2235 indicate the step may or may not be performed in combination with steps 2210-2230. Furthermore, the modification step (step 2230) may be skipped by a user and instead they may place a treatment plan order without a modification to the treatment plan. In some implementations, in response to ordering the treatment plan, the one or more processing circuits can, based on a user input, transmit to a fabrication system (e.g., 106) the treatment plan, wherein the fabrication system can be configured to manufacture a plurality of dental aligners (or other products) based on the treatment plan. In particular, the plurality of dental aligners is specific to the user and are configured to move the one or more teeth of the user according to the treatment plan. In some implementations, the one or more processing circuits can receive a user input approving the second treatment plan (e.g., the selection of a selectable object) and, in turn, the second treatment plan can be, based on the user input, transmitted to a fabrication system, where the fabrication system can be configured to manufacture a plurality of dental aligners based on the second treatment plan, and where the plurality of dental aligners are specific to the user and are configured to move the one or more teeth of the user according to the second treatment plan. In some implementations, a user input can include one or more of requesting an appointment, booking an appointment, requesting an impression kit, requesting a modification to the second treatment plan, sharing the second treatment plan with another user, placing an order, or completing a payment.

Referring to method 2250 in more detail, method 2250 includes similar features and functionalities as described with reference to method 2200. For example, steps 2260, 2270, 2275, and 2280 include similar features and functionalities as the steps of method 2200. However, at step 2265, the one or more processing circuits can generate a graphical visualization of a treatment plan. In some implementations, instead of transmitting the representation to a treatment planning system, the one or more processing circuits can execute and locally perform the functionality on the one or more processing circuits. For example, the representation can be converted into a 3D digital model, similarly to functionality described above with reference to the staging processing engine 212 of FIG. 2.

Referring now to FIGS. 23A-23G, example illustrations (2300A, 2300B, 2310, 2320, 2330, 2340, 2350) depicting a treatment plan interface, according to illustrative embodiments. The example illustrations of FIGS. 23A-23G include similar features and functionalities as described with reference to FIGS. 19A-19I, 20A-20C, and 21A-21J. For example, the user device 1902 can present the treatment plan interface 1901 in the viewport of user device 1902 which can include one or more items, objects, or types of content. Treatment plan interface 1901 can be any type of application (e.g., download from an application store, custom designed, and so on) utilized by the user of user device 1902. As shown, the treatment plan interface 1901 is shown to include a treatment plan interactive content page including a plurality of unactionable objects (e.g., text and content) and actionable (or selectable) objects (e.g., items and objects) such as buttons 2301A and 2301B, selection areas 2305, 2307, 2309, 2312, and 2313, and timeline 2303A-2303B. The treatment plan interactive content page can be presented upon selection of button 2021 of FIG. 20C. As shown, the 3D digital model (e.g., 2302A) of the user's teeth can be presented to a user to allow interactions such as viewing problems, a timeline, or different angles of the teeth. 3D digital model 2302A can be rotated and oriented to different angles based on the haptic feedback from the user. Each of the buttons 2301A and 2301B can be actionable by the user and can be configured to, upon selection, modify the view of the treatment plan interactive content page (e.g., front, top, bottom, left, right). Each of the selection areas 2305, 2307, 2309, 2312, and 2313 can be actionable by the user and can be configured to, upon selection, pop up (or open) another window (e.g., 2304, 2306, 2311, and 2314). Additionally, upon selection of areas 2305, 2307, 2309, 2312, and 2313 the pop-up windows can appear and the teeth can be updated on the treatment plan interface 1901 from a first 3D representation (e.g., final position) to a second 3D representation (e.g., initial position). In this context, the first 3D representation and the second 3D representation is a graphical user interface (GUI) object such as, but not limited to, teeth of the patient, dentition of the patient, or any other interactable item, object, or content associated with the teeth or dentition of the patient.

In some implementations, timeline 2303A-2303B can be actionable by the user and can be configured to, upon selection, update the 3D digital model 2302 (from 2302A to 2302B) based on the time period (e.g., month one, month six, new smile (after completion of the treatment plan)). For example, as shown with reference to FIG. 23A the “start” timeline 2303A is selected with an initial depiction of the dentition 2302A. In another example, as shown with reference to FIG. 23B the “new smile” timeline 2303B is selected with a final depiction of the dentition 2302B after a treatment occurs. In between the timeline 2303A-2303B the depiction of the dentition 2302 may change (from 2302A to 2302B) based on the time period selected (e.g., after one month, after six months, etc.). In some implementations, the narratives shown in the pop-up windows (e.g., e.g., 2304, 2306, 2311, and 2314) can describe features of a treatment plan and can explain some of the details of the plan. In various implementations, a selection of an interactive object (e.g., 2305, 2307, 2309, 2310, 2313) can cause the 3D representation to transform from a first 3D representation (e.g., potential dentition after treatment plan) to a second 3D representation (e.g., current dentition of the patient). For example, the first 3D representation can depict the one or more teeth of the user in a final position, and the second 3D representation can depict the one or more teeth of the user in an initial position. As shown, the transformation highlights a difference between the one or more teeth in the final position and the one or more teeth in the initial position. In some embodiments, the 3D representation can include a plurality of interactive objects (e.g., 5, 10, 15, 20 or more interactive objects) that when selected cause the 3D representation to transform from the first 3D representation to the second 3D representation. For example, each tooth may have a corresponding interactive object that causes such an effect. In some embodiments, the entire first 3D representation transforms into the second 3D representation. In some embodiments, only part of the first 3D representation transforms into the second 3D representation. For example, upon selection of an interactive object on a tooth, a region (e.g., an entire left side or right side of the dentition or teeth, immediately adjacent teeth, the tooth having the interactive object and two teeth on both sides of the tooth having the interactive object, etc.) of the first 3D representation may transform into a second 3D representation while the remainder of the first 3D representation remains the same or is modified by a second effect (e.g., grayed out, unfocused, color changed, clarity changed, moved to background, etc.). In this way, the transformation of the first 3D representation to the second 3D representation may depict the transformation that the treatment plan is intended to affect upon the user's teeth, highlighting the most significant planned changes to the user's teeth. In some embodiments, the interactive objects are selected based on a change to the user's teeth from the initial position to a final intended position meeting or exceeding a threshold requirement (e.g., a gap between teeth decreasing or increasing by a threshold amount, a tooth being moved, rotated, or extruded or intruded a threshold amount, etc.).

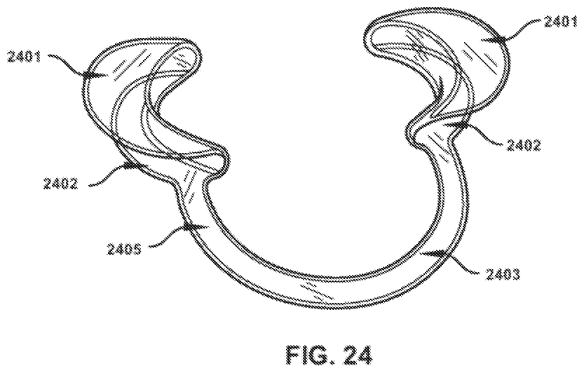

Referring now to FIG. 24, an example dental appliance 2405, according to illustrative embodiments. The dental appliance 2405 includes a handle 2403 having a pair of flanges 2401 at each end. The flanges 2401 are generally U-shaped and form a cavity 2402. An instruction manual includes appliance instructions for utilizing the dental appliance 2405. The cavity 2402 is configured to receive the user's lips at the sides of the user's mouth. The dental appliance 2405 is configured to separate the user's lips to open the user's mouth. In this position, the user may photograph his/her teeth, as described in more detail below. The dental appliance 2405 is then inserted into the user's mouth to separate the user's lips and expose the user's teeth. With the dental appliance 2405 in his/her mouth, the user takes a series of photos or videos of his/her teeth in accordance with an appliance instruction in the instruction manual. These photos or videos may then be uploaded to the vendor's website via a web portal or the like. In some implementations, the treatment plan interface 1901 may prompt the user to put in the dental appliance 2405 in response to attempting to scan the teeth (shown with reference to FIG. 19C-19D) but failing to obtain the data used to create a 3D digital model. For example, in response to presenting example illustration 2160 and the user selecting rescan button 2163, the user may be prompted to wear the dental appliances 2405 or the user device 1902 may automatically order the dental appliances 2405 to the address of the user. In some implementations, the dental appliance 2405 can be configured to hold open the user's upper and lower lips simultaneously to permit visualization (e.g., by the camera or sensor of the user device 1902) of the user's teeth and further configured to continue holding open the user's upper and lower lips in a hands-free manner after being positioned at least partially within the user's mouth. In some implementations, the flanges 2401 or the entire dental appliance 2405 may be transparent. The dental appliance is described with further reference to U.S. patent application Ser. No. 16/010,097, filed Jun. 15, 2018, the entire disclosure of which is incorporated by reference herein.

In some embodiments, the processor(s) may receive an adjustment to the final position of at least one tooth of the plurality of teeth of the dentition. The processor(s) may receive the adjustment from a treatment planning terminal 108. For example, where the second 3D representation is rendered on the treatment planning terminal 108, a user of the treatment planning terminal 108 may provide one or more adjustments to one or more teeth in the second 3D representation. The treatment planning terminal 108 may transmit the adjustment(s) to the processor(s). The processor(s) may update the second 3D representation according to the adjustment received from the treatment planning terminal 108.

In some embodiments, the processor(s) may generate a treatment plan based on the determined tooth movements of the plurality of teeth of the dentition. The processor(s) may generate the treatment plan as described above with reference to FIG. 2. The processor(s) may generate the treatment plan by generating a plurality of intermediate 3D representations of the dentition showing a progression of the plurality of teeth from the initial position to the final position. For example, the staging processing engine 212 may generate the plurality of 3D representations (e.g., the staged 3D models) which show a progression of the teeth from the initial position to the final position. In other words, the plurality of intermediate 3D representations may correspond to a respective stage of the treatment plan. The processor(s) may manufacture (or cause/trigger the manufacturing of) a plurality of dental aligners specific to the dentition and configured to move the plurality of teeth according to the determined tooth movements. For example, the processor(s) may manufacture the dental aligners by transmitting the staged 3D models (or intermediate and final 3D representations) to a fabrication computing system 106. The fabrication computing system 106 may transmit the staged 3D models to fabrication equipment to manufacture the dental aligners.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (e.g., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems, apparatuses, and methods shown in the various exemplary embodiments is illustrative only. Additionally, any element disclosed in one embodiment may be incorporated or utilized with any other embodiment disclosed herein. For example, any of the exemplary embodiments described in this application can be incorporated with any of the other exemplary embodiment described in the application. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A non-transitory computer readable medium storing instructions that, when executed by one or more processors, cause the one or more processors to:
capture a representation representing one or more teeth of a user via a camera of a user device;
transmit the representation to a treatment planning system;
receive a graphical visualization of a treatment plan for moving the one or more teeth of the user from the treatment planning system, the graphical visualization generated based on the representation;
display the graphical visualization, wherein the graphical visualization comprises:
a three-dimensional (3D) representation corresponding to the one or more teeth of the user; and
an interactive object associated with the treatment plan.

2. The non-transitory computer readable medium of claim 1, wherein the representation comprises a plurality of images of a user's dentition, wherein the plurality of images are from specific angles.

3. The non-transitory computer readable medium of claim 1, wherein the graphical visualization further comprises at least one of a view of one or more stages of the treatment plan, a view of one or more alternative treatment plans, an interactive tool for modifying the treatment plan, or a checkout option.

4. The non-transitory computer readable medium of claim 1, wherein the memory stores instructions that, when executed by the one or more processors, cause the one or more processors to:
transmit the treatment plan to a fabrication system based on a user input, wherein the fabrication system is configured to manufacture a plurality of dental aligners based on the treatment plan, wherein the plurality of dental aligners are specific to the user and are configured to move the one or more teeth of the user according to the treatment plan.

5. The non-transitory computer readable medium of claim 1, wherein the interactive object is configured to enable a selection of the treatment plan by the user device, and wherein the selection comprises at least one of purchasing the treatment plan, ordering the treatment plan, or a selection of a preferred treatment plan from among two or more treatment plans.

6. The non-transitory computer readable medium of claim 1, wherein the memory stores instructions that, when executed by the one or more processors, cause the one or more processors to:
receive a user input to adjust the treatment plan; and
transmit a request to adjust the treatment plan to the treatment planning system based on receiving the user input.

7. The non-transitory computer readable medium of claim 6, wherein adjusting the treatment plan comprises adjusting the one or more teeth in the graphical visualization, and wherein adjusting the one or more teeth further comprises updating at least one parameter of the graphical visualization.

8. The non-transitory computer readable medium of claim 1, wherein selection of the interactive object causes the 3D representation to transform from a first 3D representation to a second 3D representation.

9. The non-transitory computer readable medium of claim 8, wherein the first 3D representation depicts the one or more teeth of the user in a final position, and the second 3D representation depicts the one or more teeth of the user in an initial position.

10. The non-transitory computer readable medium of claim 9, wherein the transformation highlights a difference between the one or more teeth in the final position and the one or more teeth in the initial position.

11. The non-transitory computer readable medium of claim 1, wherein the graphical visualization comprises a planned final position of the one or more teeth after the user has completed the treatment plan.

12. The non-transitory computer readable medium of claim 1, wherein the graphical visualization is accessible via a user portal accessible via log-in credentials, and wherein the display further comprises visual indicia superimposed over the one or more teeth of the user based on the treatment plan.

13. A method of visualizing a treatment of teeth, the method comprising:
capturing, by a user device, a representation representing one or more teeth of a user via a camera of a user device;
transmitting, by the user device, the representation to a treatment planning system;
receiving, by the user device, a graphical visualization of a treatment plan for moving the one or more teeth of the user from the treatment planning system, the graphical visualization generated based on the representation;
displaying, by the user device, the graphical visualization, wherein the graphical visualization comprises:
a three-dimensional (3D) representation corresponding to the one or more teeth of the user; and
an interactive object associated with the treatment plan.

14. The method of claim 13, wherein the representation comprises a plurality of images of a user's dentition, wherein the plurality of images are from specific angles.

15. The method of claim 13, wherein the graphical visualization further comprises at least one of a view of one or more stages of the treatment plan, a view of one or more alternative treatment plans, an interactive tool for modifying the treatment plan, or a checkout option.

16. The method of claim 13, further comprising:
transmitting, by the user device, the treatment plan to a fabrication system based on a user input, wherein the fabrication system is configured to manufacture a plurality of dental aligners based on the treatment plan, wherein the plurality of dental aligners are specific to the user and are configured to move the one or more teeth of the user according to the treatment plan.

17. The method of claim 1, wherein the interactive object is configured to enable a selection of the treatment plan by the user device, and wherein the selection comprises at least one of purchasing the treatment plan, ordering the treatment plan, or a selection of a preferred treatment plan from among two or more treatment plans.

18. The method of claim 1, further comprising:
receiving, by the user device, a user input to adjust the treatment plan; and
transmitting, by the user device, a request to adjust the treatment plan to the treatment planning system based on receiving the user input.

19. The method of claim 18, wherein adjusting the treatment plan comprises adjusting the one or more teeth in the graphical visualization, and wherein adjusting the one or more teeth further comprises updating at least one parameter of the graphical visualization.

20. The method of claim 13, wherein selection of the interactive object causes the 3D representation to transform from a first 3D representation to a second 3D representation.

21. The method of claim 20, wherein the first 3D representation depicts the one or more teeth of the user in a final position, and the second 3D representation depicts the one or more teeth of the user in an initial position.

22. The method of claim 21, wherein the transformation highlights a difference between the one or more teeth in the final position and the one or more teeth in the initial position.

23. The method of claim 13, wherein the graphical visualization comprises a planned final position of the one or more teeth after the user has completed the treatment plan.

24. The method of claim 13, wherein the graphical visualization is accessible via a user portal accessible via log-in credentials, and wherein the display further comprises visual indicia superimposed over the one or more teeth of the user based on the treatment plan.

25. A non-transitory computer readable medium storing instructions that, when executed by one or more processors, cause the one or more processors to:
transmit a two-dimensional (2D) representation representing one or more teeth of a user to a treatment planning system;
receive a graphical visualization of a treatment plan for moving the one or more teeth of the user from the treatment planning system, the graphical visualization generated based on the 2D representation;
display the graphical visualization, wherein the graphical visualization comprises:
a three-dimensional (3D) representation corresponding to the one or more teeth of the user; and
an interactive object associated with the treatment plan.

26. The non-transitory computer readable medium of claim 25, wherein the representation comprises a plurality of images of a user's dentition, wherein the plurality of images are from specific angles.

27. The non-transitory computer readable medium of claim 25, wherein the graphical visualization further comprises at least one of a view of one or more stages of the treatment plan, a view of one or more alternative treatment plans, an interactive tool for modifying the treatment plan, or a checkout option.

28. The non-transitory computer readable medium of claim 25, wherein the interactive object is configured to enable a selection of the treatment plan by the user device, and wherein the selection comprises at least one of purchasing the treatment plan, ordering the treatment plan, or a selection of a preferred treatment plan from among two or more treatment plans.

29. The non-transitory computer readable medium of claim 25, wherein the memory stores instructions that, when executed by the one or more processors, cause the one or more processors to:
receive a user input to adjust the treatment plan; and
transmit a request to adjust the treatment plan to the treatment planning system based on receiving the user input, wherein adjusting the treatment plan comprises adjusting the one or more teeth in the graphical visualization, and wherein adjusting the one or more teeth further comprises updating at least one parameter of the graphical visualization.

30. The non-transitory computer readable medium of claim 29, wherein the memory stores instructions that, when executed by the one or more processors, cause the one or more processors to:
receive a second graphical visualization of a second treatment plan based on the adjustment to the treatment plan;
display the second graphical visualization, the second graphical visualization comprising:
a second three-dimensional (3D) representation corresponding to the one or more teeth of the user; and
a second interactive object associated with the second treatment plan.
